(19) United States
(12) Patent Application Publication
(10) Pub. No.: US 2024/0099934 A1
(43) Pub. Date: Mar. 28, 2024

(54) WEARABLE DEVICES, SYSTEMS, METHODS AND ARCHITECTURES FOR SENSORY STIMULATION AND MANIPULATION, AND PHYSIOLOGICAL DATA ACQUISITION AND WEARABLE HAPTIC NAVIGATION SYSTEM FOR USE IN NAVIGATING A USER AND OR POSITIONING A USER'S BODY ALONG A SAFE EGRESS PATH IN OBSCURED VISIBILITY ENVIRONMENTS

(71) Applicant: IFTech Inventing Future Technology Inc., Whitby (CA)

(72) Inventors: Brodie Myles Stanfield, Blackstock (CA); Michael Gerald Stanfield, Blackstock (CA); Alexander Ferworn, Mississauga (CA); James Elliott Coleshill, Milton (CA); Cassandra Frances Laffan, Toronto (CA); Robert Kozin, Toronto (CA)

(21) Appl. No.: 18/512,430

(22) Filed: Nov. 17, 2023

Related U.S. Application Data
(63) Continuation-in-part of application No. 18/229,528, filed on Aug. 2, 2023.
Application No. 18/229,528 is a continuation of application No. 15/108,598, filed on Jun. 28, 2016, now Pat. No. 11,759,389.
Application No. 15/108,598 is a continuation of application No. PCT/CA2014/000916, filed on Dec. 31, 2014.
(60) Provisional application No. 61/922,197, filed on Dec. 31, 2013.

(51) Int. Cl. A61H 23/02; A41D 13/12; A61B 5/00; A61F 7/02; A61H 3/06; A61H 9/00; A61H 23/04; A61H 99/00; A61N 1/04; A61N 1/32; A61N 1/36

(52) U.S. Cl. CPC A61H 23/0254; A41D 13/12; A61B 5/6804; A61F 7/02; A61H 3/061; A61H 9/0078; A61H 23/0236; A61H 23/0245; A61H 23/04; A61H 99/00; A61N 1/0452; A61N 1/0456; A61N 1/0484; A61N 1/323; A61N 1/36031; A61N 1/36034; A61B 5/0533; A61B 2562/0204; A61H 2003/063; A61H 2201/025; A61H 2201/10; A61H 2201/1614; A61H 2201/1619; A61H 2201/1623; A61H 2201/1628; A61H 2201/1635; A61H 2201/164; A61H 2201/165; A61H 2201/501; A61H 2201/5043; A61H 2201/5097; A61H 2205/06; A61H 2230/06; A61H 2230/30; A61H 2230/40; A61H 2230/50

(57) ABSTRACT

A wearable haptic navigation system for obscured visibility environments, the wearable haptic navigation system including:
a wearable haptic component, in one alternative a body covering suite; and
a mapping data collector and processor in communication with the wearable haptic component; wherein the mapping data collector and processor collects data related to a path traversed by a user of the wearable haptic navigation system and generates at least one proprioception suggestion signal to the wearable haptic component providing the user with a suggested safe egress path and/or a suggested safe body position.

This application is a continuation in part of U.S. application Ser. No. 18/229,528, filed Aug. 2, 2023, which is a continuation of U.S. application Ser. No. 15/108,598, filed Jun. 28, 2016, now U.S. Pat. No. 11,759,389, issued Sep. 19, 2023, which claims priority to International Application No. PCT/CA2014/000916, filed Dec. 31, 2014, which claims priority to U.S. Provisional Application No. 61/922,197 filed Dec. 31, 2013, the entireties of which are incorporated herein by reference.

### FIELD

Embodiments described herein relate to wearable devices, systems, methods and architectures for sensory stimulation and manipulation, and physiological data acquisition. Devices, systems, methods and architectures may be used for stimulating and manipulating the senses and physiological assessment for use in entertainment, medicine, training and education, simulation, virtual reality, research, augmented reality, augmented awareness, and so on.

### INTRODUCTION

Disaster environments, such as fires, can pose significant risks to emergency responders, particularly firefighters. These environments are often characterized by chaotic and unpredictable conditions, limited to no visibility, and a high likelihood of getting disoriented or lost while carrying out critical duties. The field of emergency response is wrought with hazards. Responders confront disasters and emergencies, facing perils both obvious, such as physical trauma from debris, to more obscure, such as getting disoriented or lost in low visibility environments. In fact, the latter is one of the most dangerous, yet deceptively common, issues a firefighter may face and it remains an unsolved problem. There are well known hazards associated with military firefights and patrols, police engagements, special weapons and tactics (SWAT) and Chemical, Biological, Radiological, Nuclear, and high yield Explosives (CBRNE) team activities, search and rescue operations on land air and sea, Emergency Medical Services (EMS), firefighting, etcetera. Some of the hazards include but are not limited to: low or no visibility environments, limited sense of direction, limited special awareness, not knowing who a combatant is, not knowing which direction the combatants are coming from or located and or not knowing where a land mine is or the direction it is located, and or becoming disoriented, and or unknowingly moving toward or entering a hazardous or toxic environment, and or the inability to communicate or being too dangerous to communicate with others, and or they may need an emergency egress, and or their current body position such as standing may put the person in danger i.e. line of fire (where they may need to change their body position to stay safe), etcetera. Traditional navigation aids often fail in these situations or do not provide solutions for these situations. Soldiers, police, EMS, firefighters, telerobotics, (unmanned aerial, water and ground vehicles) and their navigators, robots, etcetera, are having to manually determine direction and or body positioning to navigate out of dangerous environments with limited communication abilities. This is further complicated by the acute stress of the situation. This stress response can negatively affect their ability to read, hear, see, use fine motor skills, make decisions etcetera. This impact of the stress response adds to the complication of making manual decisions and following instructions auditorily, verbally, visually, or otherwise. Artificial Intelligence (AI) is becoming more prevalent in making decisions regarding comprehensive and fast paced changes in big data information including but not limited to data analytics, big data, internal and external data, primary data, secondary data, transactional data, open data etcetera. AI can be used to understand the relationships between objects and or people in a scenario.

Current wayfinding and directional systems and devices rely on existing technologies that map the earth such as geographic information systems, satellite mapping technology, geospatial technology, remote sensing, etcetera. Handheld, mobile or static devices rely on the global positioning system providing existing mapped pathways or latitude and longitude coordinates for the mobile device which then gives visual on screen and or verbal cues for directing the user or information that the user can use to follow a map. In addition, cell phones can provide vibration to alert the user. Current systems do not understand the relationships between objects and or people in a scenario. Current systems do not create maps or use created map data from other users in a workspace. Current systems do not provide spatial awareness. Current systems do not use a proprioception suggestion language. Current systems do not provide sensory cues to the individual which will stop them or change their direction or body position or point the direction of a combatant, etcetera. Current systems do not provide low or no visibility guidance (firefighters in smoke filed environments cannot see even with flashlights. There is a need for a wearable haptic system for safe navigation of a user out of a disaster environment. There is also a need for a wearable haptic system that combines lidar technology, path planning algorithms, and haptic feedback for guidance along the most efficient egress path. There is a need to minimize human decision making through an artificial intelligence guidance system that pulls, repels, directs, causes change in body positioning etcetera and automatically chooses a safe and fast route. There is a need to provide a wearable navigation system that safely directs workers from point a) to point b) and direct their body positioning through haptic and or multi-sensory cues such as pushing, pulling, stopping, bending etcetera. There is a need to provide an automated AI guidance system that determines fast and safe paths. There is a need to provide system software that can take waypoints and create maps with no prior spatial information required (no maps, schematics, etc. required) and choose fast, short, and safe pathing to traverse a workspace. There is a need to provide object detection. There is a need to provide proprioception suggestion language coding within an application, such as a software application. There is a need to provide a system that can combine the use of existing mapping technologies, with its abilities to create its own map. There is a need to provide for multiple systems communicating with each other to create and augment traversal maps providing near real-time traversability updates to each individual system supporting wayfinding within a workspace. There is a need to provide the ability of the device to recognize other devices, and their location for data transfer and mapping. There is a need to provide the ability of each individual system to obtain data input manually, visually, mechanically, auditorily using sensing devices or through other updates or communication for AI to determine safety level of current path and path choices available for providing guidance. There is a need to provide a PSL (proprioception suggestion language) that consists of a vocabulary of unique haptic signals indicating suggested changes in physical orientation (erect, bent over, crawling, etc.) and velocity, presented within a stream of suggested physical orientation (bent over, erect, etc.) and velocity changing haptic signals that can be interpreted by a receiver of the PSL as a set of suggested instructions for traversing a given shared work space (SWS). There is a need to provide the ability for the suggested instruction of the PSL to be transferred to a worker so as to indicate a direction, position, orientation, etcetera for traversing a given SWS. There is a need to provide the ability for the suggested instruction to be transferred to a worker so as to provide the ability to understand the workers body position in relation to their surroundings. There is a need to provide instructions as directional cues with a sensation(s) on the body of feeling pulled, pushed, attracted toward or repulsed, physically or otherwise directed to move by a wearable haptic navigation system. There is a need to provide sensory or physiological notification of current pathway. There is a need to provide sensory or physiological notification of direction change. There is a need to provide sensory or physiological notification of body position or change. There is a need to provide spatial awareness to the worker. There is a need to provide ability to determine fastest safest route in real time or near real time based on all data input: preexisting, created mapping, manual input, gathered from relationship awareness, etcetera.

### Electrical Stimulation

Physiological cutaneous Neuromuscular Electrical stimulation (NMES) (also referred to as powered muscle stimulation, functional muscle stimulation, and other terms), Electrical Muscle Stimulation (EMS), Transcutaneous Electrical Nerve Stimulation (TENS), Micro Current Stimulation (MC/FSM), Interferential Stimulation (IFS), Functional Electrical Stimulation (FES) and others are technologies with many different uses. Examples include but are not limited to medical and therapeutic, sports training, cosmetic, and sensory manipulation. Medical and therapeutic uses include but are not limited to: pain relief; prevention or retardation of disuse atrophy; improvement of local blood circulation, exercise of paralyzed muscles; improvement in muscle tone and strength, synchronous neuromuscular brain innervation (muscle re-education). Sports training relates to increased adaptability and outcomes for specific sporting activities as well as recuperation methodologies. Cosmetic refers to muscle toning and weight loss.

Sensory manipulation involves the manipulation of the senses by physical components embodiments described herein (referred to herein as “Sensory Manipulation”). Sensory Manipulation stimulates a person's physiology to sense various, intended and specific sensual outcomes which are associated with the real world but are only being replicated.

These stimulations may be delivered as an intermittent and repeating series of short electrical pulses but can be applied constantly for a delimited duration. Electrical outputs may be delivered transdermally by surface electrodes that are attached to a person's skin. These electrodes may be held to the skin through the use of tapes, bands, belts, straps, bonding agents, adhesives, fasteners or other mechanisms, and may contain an adjoining connector coating composed of gel or other ingredients that is capable of augmenting the efficiency of energy transfer from the electrode to the skin and subcutaneous tissues. Manual application of individual electrodes is a time consuming process that requires a high degree of accuracy and repeatability.

Different forms of currents may be used, for example; interference, diadynamic and iontophoresis. Different devices for wave forms, terminology and resultant stimulation may involve NMES, EMS, TENS, MC/FSM, IFS, and FES. Muscles may efficiently respond to electrical impulses and the frequencies generated by the devices may be important for the stimulation of slow and fast muscle fibres. Equipment used for the devices may be dynamically controlled and adjusted.

Electricity may be used as therapy, including for example NMES, EMS, TENS, MC/FSM, IFS, and FES. For example, Doctors use EMS devices for a variety of reasons. The EMS device may be especially helpful in those who are paralyzed, in pain relief, and improving blood flow in those with poor circulation. Chiropractors also use them on back injuries in order to relax the muscles, which results in faster healing times for patients.

Another example electronic stimulus application is sports. Bodybuilders claim to have received beneficial uses that help them in their weight training. These stimulation devices may also be for use during intense sport training. The devices may provide stimulation that feels comparable to weight training or explosive strength training that is used for those participating in sports that involve fast movements. They also aid in endurance and in the recovery process, post training, which decreases the chances of delayed onset of muscle soreness.

Cosmetic applications relate to helping strengthen and tone one's body. Cosmetic electrotherapy is a range of beauty treatments that uses low electric currents passed through the skin to produce several therapeutic effects such as muscle toning in the body, and micro-lifting of the face. It is based on electrotherapy which has been researched and accepted in the field of rehabilitation. Some of the therapeutic terminology used for these treatments include: Galvanic treatment, Neuromuscular Electrical Stimulation (NMES) or faradic treatment, Microcurrent Electrical Nerve Stimulation (MENS), High-frequency treatment, and so on.

Sensory Manipulation involves neuromuscular transcutaneous electrical stimulation. Sensory Manipulation occurs when a person's physiology is stimulated to sense various, intended and specific sensual outcomes which are associated with the real world but are being replicated by embodiments described herein. This form of manipulation may be used in such areas as entertainment, augmenting reality, video games, training and simulations (which also include critical incident stress disorder, CISD, and post-traumatic stress disorder, PTSD, rehabilitation programs) and video games.

There are myriad forms of electrical stimulation and varied fields of use. Each electrical stimulation device may be limited to providing a single type of stimulation to the user. In addition, reliability of outcomes may be difficult. Devices may be applied not by medically trained personnel but by home users. Therefore, manual placement of the electrodes may differ during repetitive applications.

Embodiments described herein may provide interoperability between electrical stimulation types, and the fields to which they are applied (e.g. cosmetic: face lift versus Sensory Manipulation: video game). This may include interoperability whereby the patient receives different forms of electrical stimulation concurrently through the same electrodes or simultaneously thorough separate electrodes. Embodiments described herein may provide improvements of efficiency, economy and safety. There exists a need for devices, systems, methods, and architectures for use with different forms of electrical stimulation, or at least alternatives.

### Audio

Interactivity with computers and game consoles through input devices was improved further with the introduction of haptics/tactile feedback, which may take the form of vibration feedback. Example forms of feedback for game consoles and mobile game systems, include tactile, visual and audio feedback. Surround sound technology may give a more immersive feel to an audio experience. For example, in a video game, a noise may be emitted in the same direction relative to the player as the noise relative to the player's avatar in the game. Some systems may be created to be implemented in a room, where; only a small portion defined by angles relative to the speakers can accurately include surround sound technology. This area may be referred to the “sweet-spot”. Wearable audio devices can personalize the sweet-spot to an individual user, but in the case of headphones or ear buds may be uncomfortable for long usage times, which may be the case for individuals engaging in interactive media simulation activities such as Military, Police, Fire, Hazardous Materials operations, and so on, for driving, flying and technical skill simulations. Furthermore, some devices may place pressure on the ear of those who wear glasses/eyewear causing discomfort. Moreover, they cannot be used with the various head mounted displays (“HMD”). In contrast, a device which allows the user to have force exerted on them does not appear to be available for such use. More specifically, compression stimulation may not be included in some sensory feedback systems. Accordingly, embodiments described herein may provide a device which includes wearable audio technology. In addition, embodiments described herein may provide a device that includes technology that can exert a force onto the user. There exists a need for devices, systems, methods, and architectures for use with different forms of audio technology, or at least alternatives.

### Force/Physics

Another example form of feedback is force feedback. This form of feedback is related to physics and corresponds to pushing, pulling and centripetal and centrifugal forces. This form of force feedback can be accomplished with servo-mechanisms, gyroscopes, linear actuators, and so on. A motor or series of motors built into a game controller, which may be directly or indirectly through the use of drive belts or gears are connected to game controllers control surfaces to actively oppose physical input made by the gamer. This force feedback may require more complex servo-mechanisms and controller design than passive haptic (vibration) feedback does. For example, in a steering wheel controller, force feedback may require a servo mechanism attached to the shaft of the steering wheel. Upon certain electronic commands, for example, in a very high speed turn, the servo-mechanism may act to make the steering wheel physically more difficult to turn. These various types of force feedback may be used for video games. An example type of force feedback includes gyroscopic devices integrated into hand held game controllers and joysticks. There exists a need for devices, systems, methods, and architectures for use with different forms of force feedback technology, or at least alternatives.

### Haptic Feedback

Video game controllers may incorporate haptic or tactile feedback. Vibration feedback may be accomplished by linear actuators or providing motors with offset weighting on their shafts to provide a vibration sensation when the shaft is rotated. This might be triggered, for example, to make the controller vibrate when a bomb is dropped; a car crashes; the player is struck by a bullet; etc. Game controller vibration can be tailored to offer specific tactile sensations that express the type or extent of activity occurring in the game.

Video arcade games as well as game consoles were developed and marketed to consumers. Subsequently, there has been growth in interactive multimedia, gaming, simulation training and entertainment industries synchronic with developments in computer science and technology. New developments may involve increased complexity and realism of computer-generated animation and gaming.

With gaming in particular, improvements in three dimensional (“3-D”) graphics may allow development of games with more life-like characters, realistic movements, and complex environments. The ultimate goal in some gaming programs and systems may be to enable the virtual characters therein to move and behave within the virtual environment in a natural way that emulates a physical environment as closely as possible, and to provide the user with a virtual environment that more closely simulates the experience of being in the game.

Online games, such as Massive Multiplayer Online games (MMOs), first person shooters, role playing games, racing games, adventure games, etcetera, may give users the ability to interact with multiple players in different locations around the world to enhance the strategy options, interactivity, and realism of the game.

Example interactions include visual elements of the game, two way voice

communications between the multiple players partaking within a game, the application of haptics/tactile feedback and force feedback, and so on. This remote real-time interconnectivity may provide virtual simulations and training with individuals from different locations participating together in the same virtual or real training scenario or simulation in real time. Multidirectional haptic feedback and force feedback may further enhance the end-user's entertainment and/or learning experience.

To increase the realism of a computer game further for the user, force feedback may be provided to the user in the form of muscle stimulation. As an alternative to stimulating muscles, devices may stimulate nerves (which in turn stimulate muscles). One such device is a Transcutaneous Electrical Neural Stimulation (TENS) device, and is known for use in medical applications. Force feedback devices may resist miniaturization, because they require physical motors and mechanics. In contrast, by stimulating the wearer's muscles, there may be no need for such mechanical devices which can be cumbersome.

Embodiments described herein may not only provide haptic feedback but force feedback in order to enhance Sensory Manipulation. Embodiments described herein may provide more reliable Sensory Outcomes by involving as many of the senses as possible so as to define Sensory Signatures for the user. Embodiments described herein may provide constriction/compression, temperature, airflow, sound, and so on to the user through the consistency of electrode, vibration, constriction/compression, speaker and other actuator positioning in one or multiple locals on the human body.

Embodiments described herein may provide interoperability between the multitude of stimulating types and the fields, such as video games, movies, health, augmented reality, augmented awareness, in which each or any combination may be used. In addition for the entertainment industry, training and simulation industry, gaming industry, medical and rehabilitation industry and the many implementations related to augmented awareness the embodiments disclosed herein may also provide force and haptic but may additionally include greater environmental sensual impact by allowing the wearer to feel tension, recoil, skin crawling, something brushing against them, being touched, pushed or struck and the like. There exists a need for devices, systems, methods, and architectures for use with different forms of haptic feedback technology, or at least alternatives.

### Constriction/Compression

Constriction/compression allows for but is not limited to the applying of pressure. Constriction/compression can apply pressure across an individual at a single location, multiple locations simultaneously or across entire regions of a user's body. The pressure applied can be at varying intensities and can change in intensity over time, as well as being used to create resistance to affect the individual's mobility. Constriction/compression allows for the simulating or support in the simulation of Sensory Signatures (e.g. combinations of Sensory Stimulations), as described herein. Through the use of constriction/compression the Sensory Signatures could include but are not limited to the feeling of something grabbing or holding onto the individual such as a hand; something wrapping around a part of the individual such as a bag over the shoulder; a gradual tightening feeling like a snake wrapping itself around your arm; and wearing heavy equipment and gear that is snug and tight to the body. By using constriction/compression to simulate or support in the simulation of Sensory Signatures it allows for the replication of events that otherwise could not occur.

Constriction/compression may be used in multiple fields. A few examples of its usefulness include medical, training and simulation, entertainment, and augmented awareness. The use for medical can be to provide a form of rehabilitation whereby it gently applies pressure to areas minimizing swelling and stabilizing the injury. The use in training and simulation, and entertainment may be to create a more immersive experience whereby it allows an individual to feel their gear being worn, someone grab them or moving into an obstacle such as a wall, vehicle or another person. A difference may be that, for training and simulation this can be used to create a more realistic experience while entertainment it is used to make a more enjoyable and engaging experience. As for augmented awareness this can be used to inform a user of something they cannot normally detect. In such a circumstance constriction/compression may provide different variations in pressure to the individual at different locations to inform a user of a particular change that they normally would not be aware of (i.e. in a pitch black environment whereby an individual needs to find their way through the constriction/compression may inform the individual that an obstacle is closing in from a particular direction before they even would move into it). There exists a need for devices, systems, methods, and architectures for use with different forms of constriction/compression technology, or at least alternatives.

### Temperature

An environmental sensual impact to the individual is temperature. In medicine, heat therapy, also called thermotherapy, is the application of heat to the body for pain relief and health. It can take the form of ultrasound, heating pad, cordless FIR heat therapy wrap, and many others. Heat therapy may be used for rehabilitation purposes. The therapeutic effects of heat include increasing the extensibility of collagen tissues; decreasing joint stiffness; reducing pain; relieving muscle spasms; reducing inflammation, and edema. It is useful for myalgia, fibromyalgia, contracture, bursitis and aids in the post-acute phase of healing; and increasing blood flow. The increased blood flow to the affected area provides proteins, nutrients, and oxygen for better healing.

In addition to medical applications, temperature may be an advantageous Sensory Event that would enhance Sensory Manipulation in the realm of entertainment, training and education, simulation, virtual reality, augmented reality and augmented awareness. All of these realms have temperature related environments and impacts. Some movies and video games have settings in warm environments like the heat of Africa. Military training and simulations must be appropriate to the combat environment which is currently focused on the warm desserts of the Mideast. Furthermore, temperature is a means to provide feedback to the user and can be combined with other Sensory Stimulations as part of Sensory Events to produce a Sensory Signature and the desired Sensory Outcome. There exists a need for devices, systems, methods, and architectures for use with different forms of temperature technology, or at least alternatives.

### Airflow

Air Flow allows for but is not limited to the use of temperature regulation. Temperature regulation can deal with anything from rising, lowering or maintaining an individual's body temperature. Temperature regulation may occur at one or more locations on the body to affect a particular region or could be used to alter the core temperature of the individual. Air Flow may affect an individual in such ways based on the placement of the Air Flow components, the temperature of the air flowing through the system (cool, warm, etc.) and the intensity or pressure at which the air flows through the system. Air Flow may allow for the simulating or support in the simulation of Sensory Signatures. Through the use of Air Flow the Sensory Signatures could include but are not limited to the feeling of a warm, cool or moderate breeze; a burst of air rushing passed as if they are falling from a plane or driving in a car with the window down; and a blast of air from a direction as if something exploded like a grenade. By using Air Flow to simulate or support in the simulation of Sensory Signatures it allows for the replication of events that otherwise could not occur.

Air Flow can be used in multiple fields. A few examples of its usefulness can easily be seen in medical, training and simulation, entertainment, and augmented awareness. The use for medical can be to ensure that an individual's core temperature remains within a particular range to ensure that there body is at its optimal level to help with their current condition. The use in training and simulation, and entertainment may create a more immersive experience whereby it allows an individual to feel wind, hot, cold, blast from an explosion, etc. A difference is that, for training and simulation this can be used to create a more realistic experience while entertainment it is used to make a more enjoyable and engaging experience. As for augmented awareness this can be used to inform a user of something they cannot normally detect. In such a circumstance Air Flow may provide different variations in air pressure or temperature to inform a user of a particular change that they normally would not be aware of (i.e. firefighter within a building is entering an area where carbon monoxide is detected and increasing; they are informed of the issue through the increase in air pressure from the Air Flow devices). There exists a need for devices, systems, methods, and architectures for use with different forms of air flow technology, or at least alternatives.

### Physiological Data Acquisition

An example of physiological data acquisition includes the activity of where sensors attached to your body measure key body functions. Data mining, monitoring and the interpreting of these key functions may be useful in medicine, research, training and simulations, and other fields. One area of Physiological Data Acquisition includes Electrodermal Activity (EDA). Electrodermal activity refers to electrical changes measured at the surface of the skin that arise when the skin receives innervating signals from the brain. It is a sensitive index of sympathetic nervous system activity. For most people, if you experience emotional arousal, increased cognitive workload or physical exertion, your brain sends signals to the skin to increase the level of sweating. You may not feel any sweat on the surface of the skin, but the electrical conductance increases in a measurably significant way as the pores begin to fill below the surface. This change in the ability of the skin to conduct electricity, caused by an emotional stimulus such as fright, may be called the Galvanic skin response. This response is measurable and evaluative. Other areas of Physiological data acquisition would include wireless systems such as the BioNomadix physiology monitoring devices for Electrocardiogram (ECG), Electroencephalogram (EEG), Electrogastrogram (EGG), Electromyography (EMG), Electroocoulogram (EOG), Respiration, Temperature, Pulse, Electrodermal Activity (EDA), Impedance Cardiography, Gyro, and Accelerometer. There exists a need for devices, systems, methods, and architectures for use with different forms of physiological Data acquisition technology, or at least alternatives.

### SUMMARY

The following are some definitions used herein:
a. Common off the shelf (COTS): a system that is commonly available through an arbitrary retailer or manufacturer.
b. Shared Work Space (SWS): a 3-dimensional space where activities are carried out by workers who may or may not be aware of each other.
c. Spatial Awareness: 1. The ability of a device to recognize other devices and their location for data transfer and mapping; 2. User's ability to understand their body's position in relation to their surroundings.
d. Worker/User: a human, animal or robot involved in accomplishing defined tasks within a SWS.
e. Velocity: a given speed in a given direction.
f. Safe: a state where a worked is protected from or not exposed to danger within a SWS.
g. Personal Protection Equipment (PPE): specialized clothing or equipment worn by a worker to keep safe in a SWS.
h. Path: a means of traversing from one location to another n a SWS.
i. Egress: the action of exiting a SWS.
j. Safe Egress Path (SEP): an egress path that has the characteristic of being safe.
k. Proprioception Suggestion Language (PSL): vocabulary comprising unique haptic signals indicating suggested changes in physical orientation (e.g. left, right, forward, backward, erect, bent over, crawling, walk, run, etc.,) and velocity of a user.
l. Light Detection and Ranging (LiDAR), is a method for determining ranges by targeting an object or a surface with a laser and measuring the time for the reflected light to return to the receiver.
m. Radio detection and ranging (Radar), a radiolocation system that uses radio waves to determine the distance (ranging), angle (azimuth), and radial velocity of objects relative to the site.
n. Sound navigation and ranging (Sonar), a technique that uses sound propagation to navigate, measure distances (ranging), communicate with or detect objects.
o. Inertial sensor, an electronic device that measures and reports a body's specific force, angular rate, and sometimes the orientation of the body, using a combination of accelerometers, gyroscopes, and sometimes magnetometers.
p. Echo Sounding, the use of sonar for ranging.
q. Pseudolite, a contraction of the term “pseudo-satellite,” used to refer to something that is not a satellite which performs a function commonly in the domain of satellites.
r. Ultra-wideband (UWB), a radio technology that can use a very low energy level for short-range, high-bandwidth communications over a large portion of the radio spectrum. UWB has traditional applications in non-cooperative radar imaging. Most recent applications target sensor data collection, precise locating, and tracking.
s. Ultrasonic, devices operate with frequencies from 20 kHz up to several gigahertz used to detect objects and measure distance.
t. Wireless fidelity (Wi-Fi), uses known positions of Wi-Fi hotspots to identify a device's location. It is used when GPS isn't suitable due to issues like signal interference or slow satellite acquisition. This includes assisted GPS, urban hotspot databases, and indoor positioning systems.
u. Bluetooth low energy beacons, a class of Bluetooth Low Energy (LE) devices that broadcast their identifier to nearby portable electronic devices. The technology enables smartphones, tablets and other devices to perform actions when in close proximity to a beacon to determine the device's physical location, track customers, or trigger a location-based action on the device. Another application is an indoor positioning system, which helps smartphones determine their approximate location.

According to one aspect, there is provided a wearable haptic navigation system for assisting a user through obscured visibility environments, said wearable haptic navigation system comprising:
a. a wearable haptic component; and
b. a mapping data collector and mapping data processor in communication with said wearable haptic component, wherein said wearable haptic component comprises a wearable device comprising:
c. a wearable garment
d. an input module to collect sensory related data;
e. a plurality of sensory devices connected to the wearable garment that actuate to produce one or more sensory stimulations, each of said one or more sensory stimulations for inducing physiological stimulation; and
f. a control centre comprising:
g. a processor for determining sensory events, each of said sensory events defining a synergistic action of said one or more sensory stimulations as a signal pathway to produce one or more sensory outcomes, each of said one or more sensory outcomes for inducing a physiological response or sensory perception;
h. a transceiver for receiving the sensory related data collected via the input module, and in response, sending an activating signal to actuate one or more of said plurality of sensory devices to activate the sensory events wherein the synergistic action of two or more sensory stimulations comprise at least two of electrical muscle stimulation, audio, haptic feedback, force feedback, constriction/compression, airflow, temperature stimulation and combinations thereof; wherein said mapping data collector i) collects data of a path travelled by said user in said obscured visibility environments and/or ii) retrieves pre-existing mapping data and said mapping data processor calculates from at least one of the collected mapping data and/or the retrieved pre-existing mapping data, i) a safe path for said user from a first point to a second point and ii) sends haptic signals to said wearable haptic component via proprioceptive suggestion language suggesting said safe path to said user and suggesting at least one of a safe body position, direction and speed of travel to said user.

According to one alternative, said mapping data collector is selected from the group consisting of
a. LiDAR;
b. Radar;
c. Sonar;
d. Camera;
e. environmental sensor;
f. High-definition (HD) map;
g. Inertial sensor;
h. Echosounder;
i. visible light;
j. Ultra-wideband;
k. Ultrasonic;
I. Pseudolite;
m. Wireless fidelity;
n. Bluetooth low energy;
o. Visual Simultaneous Localization and Mapping (vSLAM);
p. Infrared;
q. Thermal;
r. Low frequency magnetic waves;
s. communication system including global navigation satellite system (GNSS), assist in mapping, positioning, localization and navigation which help to determine distance speed, positioning and route guidance; and combinations thereof.

According to one alternative, said mapping data collector is a personal two-way communication device.

According to another alternative, said mapping data collector, mapping data processor and said wearable haptic component are in communication with each other via a wired, wireless and combinations thereof communication system.

According to one alternative, said wireless communication system is selected from the group consisting of Bluetooth™, Wifi, radio, satellite, mobile, wireless network, infrared, microwave, GPS, ZigBee and combinations thereof.

According to one alternative, said mapping data collector and mapping data processor further collects data of a local environment proximate said user creating a map of known wayfinding points and sends said wayfinding points to the wearable haptic component directing the user to an egress location and/or point.

According to one alternative, said wearable haptic navigation system circumvents localization issues associated with Global Positioning Systems (GPS).

According to one alternative, said wearable haptic navigation system provides physical directions to said user in a continuous direction output.

According to another aspect, there is provided a method of guiding a visibly challenged user and/or a user in an environment with obscured visibility, along a safe egress path and/or in a safe body position, said method comprising the use of the wearable haptic navigation system described herein.

According to one alternative, the method further comprises:
a. collecting data, in one alternative visual data, of a path traversed by a user, said data collected by a mapping data collector equipped device;
b. creating a traversed path from the collected data by a mapping data processor;
c. storing the traversed path;
d. determining a safe egress path and/or safe body position from the stored traversed path; and
e. communicating the safe egress path and/or safe body position to a wearable haptic component worn by said user, by proprioceptive suggestive language translated to haptic signals on the wearable haptic component urging the user to the safe egress path and/or safe body position.

According to one alternative, said haptic signals comprise directional commands, safe body position commands, velocity commands and combinations thereof.

According to one alternative, said method further comprises:
a. collecting data of at least two users in said environment.

According to one alternative, said method further comprises communicating a safe egress path and/or a safe body position to multiple users in the environment.

According to one alternative, there is provided a wearable haptic navigation system for obscured visibility environments, said wearable haptic navigation system comprising:
i. a wearable haptic component;
ii. a light detection and ranging (LIDAR) component in communication with said wearable haptic component.

According to one alternative, said wearable haptic component comprises a body covering garment.

According to one alternative, said body covering garment is that described in U.S. Pat. No. 11,759,389, herein incorporated by reference.

According to yet another alternative, there is provided a wearable haptic navigation system for obscured visibility environments, said wearable haptic navigation system comprising:
i. a haptic feedback mechanism covering all or part of a user; and
ii. a common off the shelf light detection and ranging enabled communication device, such as a mobile phone or the like, in communication with said haptic feedback mechanism.

According to yet another alternative, said mapping data collector and LIDAR component is a software program.

According to yet another alternative, said LIDAR component is a LIDAR enabled device.

According to yet another alternative, said LIDAR enabled device is a two-way communication device, such as, but not limited to, a smart phone.

According to yet another alternative, said software program is an Apple iPhone LIDAR software application.

In one alternative, said LIDAR component and said wearable haptic component are in communication with each other via an arbitrary wireless system.

In one alternative, said arbitrary wireless system is selected from the group consisting of Bluetooth, WiFi, radio, satellite, mobile, wireless network, infrared, microwave, GPS, ZigBee and combinations thereof.

In one alternative, said wearable haptic component is worn under any torso covering personal protective equipment (PPE) of a user.

In another alternative, said mapping data collector and/or LIDAR component is situated at a forward-facing area of the PPE of the user.

In one alternative, said mapping data collector and/or LIDAR component collects data of the local environment proximate said user creating a map of known wayfinding points and sends said wayfinding points to the wearable haptic component directing the user to an egress location and/or point.

In another alternative, said wearable haptic navigation system provides physical directions to the user in a visibly-denied environment.

In one alternative, said wearable haptic navigation system provides physical directions to the user in a visibly-denied environment while circumventing localization issues associated with Global Positioning Systems (GPS).

In one alternative, said wearable haptic navigation system provides physical directions to the user in a visibly-denied environment with continuous directional output.

In one alternative, said wearable haptic navigation system incorporates proprioception suggestion language for haptic signals in the wearable haptic component as suggestive instructions to traverse a given shared workspace.

According to yet another aspect, there is provided a method of guiding a user, that is visibly challenged or that is in an environment with obscured visibility, along a safe egress path, said method comprising the use of the wearable haptic navigation system as described herein.

In one alternative, said method further comprises:
a. collecting data, in one alternative visual data, of a path traversed by a user, in one alternative said data collected by a LIDAR equipped device;
b. creating a traversed path from the collected data;
c. storing the traversed path;
d. determining a safe egress path from the stored traversed path; and
e. communicating the safe egress path to a wearable haptic component, in one alternative a wearable haptic torso vest, worn by said user, by proprioceptive suggestive language translated to haptic signals on the wearable haptic component urging the user to the safe egress path.

In one alternative, said haptic signals comprise directional commands, position commands, velocity commands and combinations thereof.

In one alternative, said method further comprises collecting data of at least two users in said environment.

In another alternative, said method further comprises communicating safe egress paths to multiple users in the environment.

According to another aspect, there is provided a wearable haptic navigation and body positioning system comprising:
a. a haptic feedback mechanism in communication with
b. a mapping data collector and processor.

In one alternative, the mapping data collector and processor periodically collects environmental scans of a space creating an internal map of known wayfinding points in three-dimensional space representing a path travelled by a user of the system.

In another alternative, the mapping data collector receives mapping data from a source other than from the wearable haptic navigation and body positioning system.

In another alternative, said wearable haptic navigation system is used in law enforcement, emergency medical services and firefighting.

In another alternative, said wearable haptic navigation system is used in actual flight and flight simulation.

In another alternative, said wearable haptic navigation system is in combination with a flight simulation system.

Proprioception is the body's ability to sense its location, movements, and actions within its given environment; this includes situational and spatial awareness. When we move, our brain senses the effort, force, and heaviness of our actions and positions and responds accordingly. It encompasses a complex of sensations, including perception of joint position and movement, muscle force, and effort. This is the reason we are able to move freely without consciously thinking about our environment. Proprioception is basically a continuous loop of feedback between sensory receptors throughout your body and your nervous system. Sensory receptors are located on your skin, joints, and muscles.

Examples of proprioception include being able to walk or kick without looking at your feet or being able touch your nose with your eyes closed bend over, crouch down, etcetera. Some things can affect proprioception. Temporary impairment can come from drinking too much alcohol, which is why a sobriety test involves touching your nose while standing on one foot. Sight impairment, hearing impairment and or injuries or medical conditions that affect the muscles, nerves, and the brain can also cause immediate temporary, long term or permanent proprioception impairment.

The ability to navigate space in our physical world relies on our body organizing and integrating information from the visual (eyes), proprioceptive (information perceived through our muscles and joints to tell us where we are in space) and vestibular (inner ears sensing motion, equilibrium and spatial awareness) systems. A deficiency in any one of these three vitals systems can have a dramatic impact on the situational awareness, spatial awareness, person's ability to move in their world.

There exists a need for devices, systems, methods and architectures to assist our body movement(s) when proprioception is compromised.

Acute stress often impacting those in chaotic environments or emergency situations can cause the stress affect resulting in negative reactions of the body. Some of the physiological reactions experienced in acutely stressful environments include audio exclusion (not hearing all relevant sounds or noises), perception narrowing or tunnel vision (tendency for vision and perceptional field to shrink putting focus on most dangerous item, i.e. weapon or fire, which can mean missing other details or threats), time distortion which makes the event or activities appear to slow down or speed up; awareness lapse where certain portions of the event are not recalled because the brain is focused on possible danger and threats may not process all information in the environment, Loss of fine motor skills and dexterity or skills involving refined use of small muscle control, confusion, etcetera.

These physiological responses to stress limit situational awareness. This limitation includes the reduction of one's ability to perceive, comprehend and understand the surrounding environment including a lack of understanding the distance and relationship to and between things within the environment.

Such as military personnel pinned down, taking heavy artillery, gun fire and casualties. The order is given to fall back. A low body position profile is required, and a specific direction is required to get to the safe fall-back area. Soldiers not adhering to a low profile (crouching down) or those who take a longer path to safety could be injured or killed. Here, in one alternative, the wearable haptic navigation system determines the fastest and safest path, utilizes PSL and stimulates the necessary nerves and muscles in the legs and abdomen of the user to cue the user to crouch down and slightly bend forward. In parallel with body positioning the PSL will signal to the user the direction or path to follow and where necessary stopping, redirecting or changing the direction, body position (standing, crawling, bent over, etcetera) of the user.

Another scenario is firefighters have gone into the subway due to what appears to be a subway accident. There is heavy smoke and casualties. It is a terrorist event and a secondary device is triggered. Personnel must now find the nearest egress to safety.

The wearable haptic navigation system provides a means of communication and a mechanism that responds to this communication which can be used to direct the body of those who are experiencing difficulty in these situations to the safest body position, direction, or egress path to a new location or past location from their current location or to avoid objects and/or dangerous areas, when necessary.

Spatial disorientation is the inability of a person to determine his true body position, motion, and altitude relative to the earth or his surroundings. The inability to determine position or relative motion, commonly occurring during periods of challenging visibility, since vision is the dominant sense for orientation. The auditory system, vestibular system (within the inner ear), and proprioceptive system (sensory receptors located in the skin, muscles, tendons and joints) collectively work to coordinate movement with balance, and can also create illusory nonvisual sensations, resulting in spatial disorientation in the absence of strong visual cues.

In aviation for example, spatial disorientation can result in improper perception of the altitude of the aircraft, referring to the motion of the aircraft (whether turning, ascending or descending). For aviators, proper recognition of aircraft altitude is most critical at night or in poor weather, when there is no visible horizon, and spatial disorientation has led to numerous aviation accidents. Spatial disorientation can occur in other situations where visibility is reduced, such as diving operations, driving a vehicle in inclement weather, such as but not limited to fog or snow, etcetera. For example, an inexperienced air pilot encounters weather that forces the air pilot to fly by instruments alone. The air pilot loses visual reference to the ground. The limited experience of flying by instruments coupled with the inability to see the horizon or have any reference to the ground causes spatial disorientation and the air pilot does not maintain the proper altitude which can be disastrous. In another scenario an air pilot, due to a gradual turn of the plane, does not realize the plane is ascending and needs to bring the plane back to its original altitude, or the air pilot goes into a rapid roll where the air pilot becomes disoriented due to the continued feeling of leaning caused by the initial inertia of the roll even after the motion has stopped.

The present wearable haptics navigation system provides a means of communication and a mechanism that responds to this communication which provides an indication to the body of those who are experiencing spatial disorientation such as their relative position or motion and indicate any changes that should be made to their body position, direction, or path from their current positioning which assists in reorienting them when necessary. The present wearable haptics navigation system provides provides or assists proprioception or at least assists in maintaining this sense at its highest ability in order to allow personnel to reorient themselves regaining spatial awareness.

### Low or No Visibility Environment

Many workers including police, fire fighters, paramedic and ambulance workers, military and civilian security personnel, confined space specialists, mining operations personnel, find themselves with limited, low or no visibility during their operations. This lack of visibility issue may be due to dust, smoke, darkness, vison loss, injury, vegetation, dirty windows, toxic gas, direct sunlight and other causes. Visibility may also be limited due to mitigation of danger such as not putting oneself in the line of fire in order to get a line of sight (police officer pinned down behind a wall or car does not get up to see where the fire is coming from because they might get shot). During these high-risk operations these personnel may find themselves in situations where they may need to evacuate quickly, move to another area, return fire, crawl or crouch due to heat or other danger, bend down, run, step up or down for stairs or any movement to avoid objects or dangers such as a firefighter on the 2nd floor of a two-story townhouse building in a smoke-filled environment. The firefighter has been in the building moving and searching for 30 minutes and the signal is heard to evacuate due to imminent collapse. The firefighter may only have a few minutes and must now try to determine how to get out and which direction to move. They can become disoriented not knowing which direction to move or what is in front of them. They must use their hands to feel the walls, maneuver around furniture and obstructions, and to determine when to bend down, stand up or crawl on the floor. If they find a fire hose, they can try to follow it hoping it leads out of the building and not further into the fire. This takes time is fraught with danger and is extremely stressful.

Another scenario is a police officer in an underground parking garage who is fired upon and does not know where the perpetrator is located. He could stand up and look around to try and see where the perpetrator and the shots are coming from but in doing so risks being shot.

When the need to respond quickly to change body position, relocate, return fire etcetera is demanded, the wearable haptic navigation system provides a means of communication and a mechanism that responds to this communication which can be used to swiftly direct the body of those who are experiencing low or no visibility to the safest body position (crouching, crawling, stepping up, bending over, etcetera), direction (facing or aiming, rotating, etcetera forward backward, up or down etcetera), or egress path to a new location, past location from their current location or to avoid objects, dangerous areas, have their body positioned in the direction of the perpetrator in order to return fire, when necessary. The wearable haptic navigation system provides and/or assists proprioception toward performing the safest movement possible.

The wearable haptic navigation system assists our body movement(s) when proprioception is compromised. The wearable haptic navigation system includes a means of suggesting or proposing those compromised movements and actions as well as delivering signals which can be understood and executed by the body in order to assist in or produce the movements. Proprioception language is the continuous feedback loop of communication between sensory receptors throughout the body including our brain skin, joints, muscles etcetera. Multisensory stimulation, haptics, force feedback, constriction, EMS are well known for their use in producing effects on the body which can stimulate muscles or force movement of body parts around its joints.

A Proprioception Suggestion Language (PSL) consists of a vocabulary of unique haptic or sensory signals indicating suggested changes in physical orientation (erect, bent over, crawling, twisting, etc.,) and velocity presented within a stream of suggested physical orientations (bent over, erect, etc.,) and velocity changing haptic signals that can be interpreted by a receiver of the PSL as a set of suggested instructions for traversing a given shared workspace.

Artificial Intelligence (AI) or machine learning may be used to assist in decision making. There are areas where decisions for personnel or taking action can be very difficult due to environmental circumstances and their impact on us. In these circumstances it may be advantageous to allow AI to assist in decision making or for AI to make the decision itself. AI is a data driven decision making system that enables machine learning to think rationally and act rationally. The discipline is made up of algorithms designed to make choices and based on the data and ensuing results learn from those choices. In situations where humans are unable to make choices/decisions due to environmental (such as low visibility or no visibility), physiological (acute stress, injury, etcetera) or other circumstances, AI can assist in making these choices/decisions. Some examples include Chat GBT, AI Bing, Google Duplex, Google assistant, autonomous vehicles, google maps etc.

Artificial Intelligence (AI) reviews maps created, existing maps, directional cues based on all data received, any manual input and all other data collected. AI looks at path and data metrics of physical orientation requirements at specific datapoints. AI uses PSL to configure unique haptic/sensory signals along path. AI adjusts path and PSL to provide the safest movement and or shortest route to the required destination. PSL signals are provided to the wearable haptic navigation system in real time. AI is also used to recognize things through sensors and discern what it is, such as people, animals, vehicles, furniture etcetera, this is known as object detection. As an example Wifi, passive infra-red, thermal imaging, face recognition, etcetera are means to determine if an object is human. Wifi has also been used by researchers at Carnegie Mellon University to map human bodies through walls as a cheap alternative to other more expensive methods. Weapons can also be detected by AI such as the ZeroEyes software using cameras, other examples of sensors used to detect weapons include infrared sensor cameras, and a combined fused imagery of visual and infrared images.

In an aspect, embodiments described herein may provide a wearable device comprising a wearable garment and an input module to collect sensory related data. Sensory Devices connect to the wearable garment that actuate to produce Sensory Stimulations, each Sensory Stimulation for inducing physiological stimulation. A Control Centre has a processor for determining Sensory Events, each Sensory Event defining a synergistic action of one or more Sensory Stimulations as a Signal Pathway to produce one or more Sensory Outcomes, each Sensory Outcome for inducing a physiological response

or sensory perception, and a transceiver for receiving the sensory related data collected via the input module, and in response, sending an activating signal to actuate one or more Sensor Devices of the plurality of Sensor Devices to activate the Sensory Events.

In another aspect, embodiments described herein may provide interoperable wearable devices which can be used between fields and disciplines, such as electrical stimulation, audio, force feedback, haptic feedback, constriction/compression, temperature, air flow, physiological data acquisition, and so on.

In another aspect, embodiments described herein may provide devices, systems, methods, and architectures that may provide a synergistic action of multiple Sensory Stimulations through audio, EMS, haptic feedback, force feedback, constriction/compression, airflow, temperature, and so on.

In a further aspect, embodiments described herein may provide devices, systems, methods, and architectures that may provide Sensory Stimulation and Sensory Manipulation through activation of Sensory Events. The Sensory Stimulations create Sensory Signatures that may provide intended Sensory Outcome(s), and so on.

In another aspect, embodiments described herein may provide devices, systems, methods, and architectures that may receive immediate physiological feedback data, and implement various Sensory Outcome(s), and so on.

In another aspect, embodiments described herein may provide wearable device comprising: wearable material; an input module to collect sensory related data; electrical stimulus interfaces (electrodes) connected to the wearable material wherein the electrical stimulus interfaces actuate to provide Sensory Manipulation to activate Sensory Events in response to sensory related data collected via the input module to create a variety of Sensory Outcomes.

In accordance with some embodiments, the wearable device may further include a Control Centre having a transceiver which determines the Sensory Manipulation and actuates the electrical stimulus interfaces.

In accordance with some embodiments, the wearable device may further include a Decoder to collect raw data from the input module, the data being sent from an initiating device and transform the data into a format compatible with the Control Centre, wherein the Decoder transmits transformed data via a communications protocol to the Control Centre.

In accordance with some embodiments, the Control Centre translates raw data from the Decoder into Sensory Stimulation(s) for Sensory Manipulations.

In accordance with some embodiments, the Control Centre stores Personalized Settings to determine maximum and minimum sensations for Sensory Manipulations.

In accordance with some embodiments, the input module collects data from sensor devices.

In accordance with some embodiments, the Control Centre is the component of the device which controls the signal, duration, strength, and/or pattern of the electrical stimulus generated causing a Sensory Event, whether singularly, in a Sensory Event Array, random or other formation.

In accordance with some embodiments, the electrodes are removable from the wearable material.

In accordance with some embodiments, the Control Centre is removable from the wearable material.

In accordance with some embodiments, the electrodes are actuators for the force, constriction/compression, vibration and electrical stimulation.

In accordance with some embodiments, the Control Centre selectively identifies which areas of the wearable material are to be activated.

In accordance with some embodiments, the electrodes can deliver multiple variations of stimulation including, but not limited to: Electrical Muscle Stimulation (EMS), Transcutaneous Electrical Nerve Stimulation (TENS), Micro Current Stimulation

(MC/FSM), Interferential Stimulation (IFS), Functional Electrical Stimulation (FES) and Neuromuscular Electrical Stimulation (NMES).

In accordance with some embodiments, the Sensory Manipulation(s) provided by the actuated electrodes may occur singularly or in any combination: synchronous, intermittent, consecutive, and imbricate.

In accordance with some embodiments, predetermined and defined electrode placement is based on Sensory Signature(s).

In accordance with some embodiments, positions of the electrodes are user adjustable and the wearable material can optionally have indicators detailing position(s) of electrode(s) to facilitate accurate placement.

In accordance with some embodiments, there may be a set number of allowable locations for the electrodes within the wearable material.

In accordance with some embodiments, positions of the electrodes are predetermined according to neuromuscular and medical indications and recommendation.

In accordance with some embodiments, the wearable device may further include an audio Decoder to collect audio data from the initiating device, the data being taken from an initiating device and transferring the data via a communications protocol to amplifier/transmitter/receiver.

In accordance with some embodiments, the wearable device may further include speakers to provide Individualized Local Sound.

In accordance with some embodiments, the wearable device may further include amplifier/transmitter/receiver that is operatively connected to the input module (or initiating device) through the audio Decoder to receive, amplify and transmit the incoming data to the speakers.

In accordance with some embodiments, the wearable device may further include vibration actuators.

In accordance with some embodiments, the wearable device may further include force simulation device actuators that may apply physical forces to an individual so that they feel particular sensations that would normally pertain to a particular real-world event.

In accordance with some embodiments, the wearable device may further include force simulation device actuators that may apply localized forces.

In accordance with some embodiments, the force simulation device actuators may alter actuated force based on such parameters as the amount of force that is applied (minimal to maximum), the speed at which the force reaches its target amount (fast or slow), the duration to which the force is applied (amount of seconds or deactivates once target force is reached) and the speed at which the force is removed (fast or slow).

In accordance with some embodiments, the wearable device may further include Constriction/Compression Stimulation Device actuators that provide capabilities of applying a compression and/or constrictive feeling to a location of an individual's body.

In accordance with some embodiments, the Constriction/Compression Stimulation Device actuators may alter actuated constriction/compression based on various parameters altered to effect the sensation of constriction/compression and squeezing such as but not limited to the pressure (minimal or a lot), tightening (minimal or a lot), speed that squeezing or constriction/compression occurs or is removed (fast or slow), the length the constriction/compression is activated for (multiple seconds or once fully activated revert to deactivated state) and the ability to fluctuate between these settings while already activated.

In accordance with some embodiments, the wearable material is separated into three garment areas of the body, one being the abdominal area, one being the upper torso or chest and shoulder area, and one representing coverage of both the abdominal and torso area figures, wherein all components may be interconnected to provide synergy and totality of Sensory Manipulation throughout the entire garment as defined by a Signal Pathway to create the Sensory Signatures which produce a desired Sensory Outcome.

In a further aspect, embodiments described herein may provide a wearable device comprising wearable material, electrodes, a Medically Compliant Electrical Impulse Amplifier Transmitter Receiver (MCEIATR), and a control center, wherein the control center actuates the MCEIATR which in turn provides the stimulus through the electrodes positioned on the wearable material.

Additional example embodiments are described herein.

### BRIEF DESCRIPTION OF THE DRAWINGS

The embodiments will now be described by way of example only, with reference to the accompanying drawings, in which:

FIG. 1 is a schematic representation of the connection between the MCEIATR and a pair of electrodes;

FIG. 2 is a schematic representation of a pair of predetermined and defined electrodes within a garment;

FIG. 3 is a schematic representation of a garment;

FIG. 3a is a schematic representation of a garment with a plurality of electrodes and showing all electrodes activated, where the electrodes are wired directly to the MCEIATR;

FIG. 3b is a schematic representation similar to that shown in FIG. 3a but showing some of the electrodes activated, where the electrodes may not be wired and may utilize a conductive garment whereby the MCEIATR is connected to the conductive garment and the electrodes are connected to the conductive garment. These are garments that may be wireless taking the charge through the garment and sending it to the electrode. In another embodiment the garment may be made of specific materials that do not require electrodes. These garments may plug directly into the MCEIATR. In the embodiments denoted by FIGS. 3b and 3c “conductive garments” may be used to transfer the electrical charge to and from the electrodes.

FIG. 3c is a schematic representation similar to that shown in FIGS. 3a and 3b but showing none of the electrodes activated;

FIG. 4a is a schematic representation of an audio architecture relative the user;

FIG. 4b is a schematic representation of an embodiment with a plurality of speakers surrounding the user;

FIG. 4c is a schematic representation of the front of the user with the embodiment of 4b;

FIG. 4d is a schematic representation of the back of the user with the embodiment of 4b;

FIG. 5a is a schematic representation of the interior of the first layer of the garment of an embodiment;

FIG. 5b is a schematic representation of the interior and/or exterior of the second layer of the garment of an embodiment and includes the placement of the Control Centre and power regulator on the exterior of the second layer of the garment;

FIG. 5c is a schematic representation of the third layer of the garment of the embodiment;

FIG. 6 is a schematic representation of an embodiment with electrodes attached to the first layer of the garment;

FIG. 7 is a schematic representation of an embodiment with vibration actuators attached to the second layer of the garment;

FIG. 8 is a schematic representation of an arrangement the speakers and their location on the garment;

FIG. 9 is a schematic representation of an embodiment with electrodes attached to the first layer of the garment;

FIG. 10 is a schematic representation of an embodiment with Constriction/Compression Stimulation Device actuators on the second layer of the garment;

FIG. 10a is a schematic representation of an embodiment with Force/Physics Simulation Device actuators on the second layer of the garment;

FIG. 11 is a schematic representation of an embodiment with MCEIATR attached to the exterior of the garment;

FIGS. 12a to 12i are schematic representations of arrays of actuators, which may be referred to as a “Sensory Event Array,” and each representation shows a different example combination of actuators being actuated either simultaneously in sequence or a combination of both;

FIG. 13a is a schematic representation of the frontal view of an embodiment with individual surround sound (which may referred to as Two Way Voice and Multidirectional Audio Communication Interactive Media Device) (item 41 in FIG. 14) Wearable Predetermined Electrical Stimulation Technology (WPEST), vibration actuators, force/compression actuators; this view is meant to be seen as if the garment were see through. This shows the different components that exist and does not show that they are on different layers of the suit. This is to show the different components that exist but does not include overlap as this would obscure from view those things underneath. Therefore, the componentry configuration shown is designed as a representation of the embodiment but the actual design may contain a greater number of Sensory Devices than shown here.

FIG. 13b is a schematic representation of a side view of the embodiment shown in FIG. 13a; and also like FIG. 13a this view of an example embodiment is meant to be seen as if the garment were see through.

FIG. 13c is a schematic representation of a frontal view of an embodiment which includes the entire body; the entire torso including the arms and waist, the lower body including the hips, upper and lower legs. Also like FIG. 13a this view of an example embodiment is meant to be seen as if the garment were see through.

FIG. 14 is a schematic representation of the signal pathway from the initiating device to actuator and from the actuator to the initiating device.

FIG. 15 illustrates Decoder specifications according to some embodiments.

FIG. 16 illustrates exoskeleton specifications according to some embodiments.

FIGS. 17a and 17b illustrate Control Centre specifications according to some embodiments.

FIG. 18 illustrates nervous system specifications for power activation according to some embodiments.

FIG. 19 illustrates nervous system specifications for vibration according to some embodiments.

FIGS. 20a through 20d illustrates nervous system specifications for surround sound according to some embodiments.

FIG. 21 illustrates power regulation specifications according to some embodiments.

FIG. 22 illustrates wearable material specifications according to some embodiments.

FIG. 23 illustrates an example gaming console architecture according to some embodiments.

FIG. 24 illustrates example nervous system Smart Transducer Interface Modules (STIMS) specifications. The STIMS includes MCEIAs and paired electrodes.

FIGS. 25 to 37 illustrate sensory device placement for example embodiments.

FIG. 38 is an overhead view of the wearable haptic navigation system in use, according to one alternative.

FIG. 39 depicts the wearable haptic component with selected haptic signals activated, according to one alternative.

FIG. 40 depicts directional control of the wearable haptic navigation system, according to one alternative.

FIG. 41 depicts the paths travelled, according to Example 1.

FIG. 42 depicts the communication between the LIDAR equipped smartphone and the wearable haptic component, according to one alternative.

FIGS. 43(a), (b) and (c) depict a law enforcement scenario, EMS scenario and firefighting scenario, respectively.

FIG. 44 provides a flight simulator in combination with the wearable haptic navigation system, in one alternative.

FIG. 45 provides a flight simulator in combination with the wearable haptic navigation system in another alternative.

### DETAILED DESCRIPTION OF VARIOUS EMBODIMENTS

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example, and without limitation, the various programmable computers may be a server, network appliance, set-top box, embedded device, computer expansion module, personal computer, laptop, personal data assistant, cellular telephone, smartphone device, ultra mobile PC (UMPC) tablets and wireless hypermedia device or any other computing device capable of being configured to carry out the methods described herein.

Program code is applied to input data to perform the functions described herein and to generate output information. The output information is applied to one or more output devices, in known fashion. In some embodiments, the communication interface may be a network communication interface. In embodiments in which elements of the invention are combined, the communication interface may be a software communication interface, such as those for inter-process communication. In still other embodiments, there may be a combination of communication interfaces implemented as hardware, software, and combination thereof.

Each program may be implemented in a high-level procedural or object oriented programming or scripting language, or a combination thereof, to communicate with a computer system. However, alternatively the programs may be implemented in assembly or machine language, if desired. The language may be a compiled or interpreted language. Each such computer program may be stored on a storage media or a device (e.g., ROM, magnetic disk, optical disc), readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the systems and methods of the described embodiments are capable of being distributed in a computer program product including a physical, non-transitory computer readable medium that bears computer usable instructions for one or more processors. The medium may be provided in various forms, including one or more diskettes, compact disks, tapes, chips, magnetic and electronic storage media, volatile memory, non-volatile memory and the like. Non-transitory computer-readable media may include all computer-readable media, with the exception being a transitory, propagating signal. The term non-transitory is not intended to exclude computer readable media such as primary memory, volatile memory, RAM and so on, where the data stored thereon may only be temporarily stored. The computer useable instructions may also be in various forms, including compiled and non-compiled code.

Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. One should appreciate that the systems and methods described herein may provide various technical effects. For example, embodiments may include tangible actuate electrical stimulus interfaces (electrodes) to provide tangible stimulation in response to activated Sensory Events. The activations or actuations of specific Sensory Devices of the Nervous System may translate into tangible Sensory Stimulation to provide physiological stimulation for the user. As an example, a Force Simulation Device may apply physical forces to an individual so that they feel particular sensations that would normally pertain to a particular real world event. Sensory Stimulations include audio, vibration, electrical stimulation, force/physics, constriction/compression, and so on. A Force Simulation Device may allow for virtual mediums to have an increased immersive experience as a force applied to the body will give the intensity of the force applied and the direction to which the force came from based on its location in the garment. Sensory related data may be collected in raw data form and transformed by hardware components into data representative of different sensory experiences and stimulations, as described herein. Other example technical effects are described herein.

The following discussion provides many example embodiments. Although each embodiment represents a single combination of inventive elements, other embodiments may represent all possible combinations of the disclosed elements. Thus, if one embodiment comprises elements A, B, and C, and a second embodiment comprises elements B and D, then other embodiments may include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Various terms and definitions used herein will be described herein to enhance clarity and facilitate illustration of various example embodiments. These are example descriptions for illustrations.

Computing Devices may be used herein to relate to an electronic device that sends and/or receives data to initiate and/or activate the particular componentry that we discuss in this patent. Such as, but not limited to, any form of computer. Computing Devices may be operable by users to access functionality of embodiments described herein. Computing Devices may be the same or different types of devices. Computing Devices may be implemented using one or more processors and one or more data storage devices configured with database(s) or file system(s), or using multiple devices or groups of storage devices distributed over a wide geographic area and connected via a network (which may be referred to as “cloud computing”). Computing Devices may reside on any networked computing device, such as a personal computer, workstation, server, portable computer, mobile device, personal digital assistant, laptop, tablet, smart phone, WAP phone, an interactive television, video display terminals, gaming consoles, electronic reading device, and portable electronic devices or a combination of these. Computing Devices may include any type of processor, such as, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. Computing Devices may include any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Computing Devices may include one or more input devices, such as a keyboard, mouse, camera, touch screen and a microphone, and may also include one or more output devices such as a display screen and a speaker. Computing Devices may have a network interface in order to communicate with other components, to access and connect to network resources, to serve an application and other applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these. There may be more Computing Devices distributed over a geographic area and connected via a network. Computing Devices is operable to register and authenticate users (using a login, unique identifier, and password for example) prior to providing access to applications, a local network, network resources, other networks and network security devices. Computing Devices may be different types of devices and may serve one user or multiple users.

Interoperability may be used herein to refer to the ability of wearable technologies in accordance with embodiments described herein to be utilized across fields and disciplines to work with other systems without special effort on the part of the customer.

Medically Compliant Electrical Impulse Amplifier Transmitter Receive (MCEIATR) may be used herein to relate to a computing device that is intended to provide stimulation to the physiology through the application of electrical energy to the physiology; to receive data from the physiology; and to transmit data wirelessly. This may be a device that is medically compliant in its activation protocol and limitations as well as adheres to the US FDA standards for such devices and which includes over the counter and prescription units. These devices emit an electrical pulse that may be transferred through electrodes and or conductive fabric and transcutaneously through the wearer's physiology attaining the designated results. Furthermore, these devices may receive data through electrodes and or conductive fabric acquiring physiological information of the wearer. The MCEIATR may be defined in the garment or can be external.

Operably Connected may be used herein to refer to any components that are directly or indirectly connected or coupled. Any form of connection that allows for communication between the components is allowable. This includes but is not limited to; wired connections, wireless, Wi-Fi, MILAN (wireless local area network), radio, near-field communication, or Bluetooth™ connections or a combination thereof.

Nervous System may be used herein to refer to all the componentry that is attached or connected to the Control Center that works to provide or produce Sensory Stimulation(s) to the wearer and more specifically can refer to the Sensory Devices and their integration as a whole.

Sensory Device (SD) may be used herein to refer to any contrivance, such as an ultrasonic pad or electrode, that receives and or responds to data, a signal or stimulus and translates or transfers this input into a form of energy that acts on one or more of the faculties by which the body perceives an external stimulus; one of the faculties of sight, smell, hearing, taste, and touch. Sensory Device actuates to produce Sensory Stimulations to act on the body faculties as physiological stimuli.

Sensory Stimulation may be used herein to refer to the activation of one or more of the body's faculties of sight, smell, hearing, taste, and touch through the actuation of one or more Sensory Devices. Different types of Sensory Stimulation may produce different types of physiological stimulation.

Sensory Manipulation may be used herein to refer to the use of a Sensory Device(s) to provide Sensory Stimulation(s) for a specific purpose or outcome. Sensory Devices may actuate to produce Sensory Manipulations as one or more Sensory Stimulations.

Sensory Event may be used herein to relate to any single or simultaneous Sensory Device (SD) activation which produces Sensory Stimulations or Sensory Manipulation. In addition, Sensory Event refers to the synergistic action of multiple Sensory Stimulations of different types such as through audio, EMS, haptic feedback, force feedback, constriction/compression, airflow, temperature and so on to produce a desired Sensory Signature and/or Sensory Outcome. A Sensory Event may contain one or more simultaneous Sensory Stimulation activations as a Sensory Manipulation. A Sensory Event occurs when a computing device or Control Centre sends an activating signal to one or more Sensory Devices actuators which produce Sensory Stimulations and stimulates the user's physiology. More than one type of Sensory Device may be actuated during a Sensory Event. More than one type of Sensory Stimulation may be produced during a Sensory Event. A Signal Path may define a Sensory Event to indicate a set of Sensory Devices to actuate and a set of Sensory Stimulations to produce using the actuated Sensory Devices. A Sensory Event may involve simultaneously or sequential actuation of Sensory Devices to produce different patterns of Sensory Stimulations, as a Sensory Signature or Sensory Outcome.

Sensory Event Array may be used herein to refer to the dispersal pattern of Sensory Stimulation through the combination of simultaneous and or sequential Sensory Event activations and Sensory Device actuations.

Sensory Signature may be used herein to refer to sensory information outputs that a particular object manifests to be recognizable and perceived through the user's senses. This may be enhanced by situational awareness (such as knowing what type of environment they are in, such as for example Realistic Military Simulation or Sci-Fi world). A Sensory Signature may be produced through the application of specific Sensory Events which provide intended Sensory Manipulation (e.g. Sensory Stimulations by actuation of Sensory Devices) to produce the reality within the user's mind as portrayed in the virtual world. It is the specific and reproducible induced physiological response outcome (e.g. Sensory Outcome) of the user created through Sensory Manipulation. It may be achieved utilizing a specific and defined set of Sensory Stimulations and Sensory Device activations as defined in the specified Sensory Event.

Sensory Outcome may be used herein to refer to the user's physiological response to a Sensory Event or Sensory Signature(s) applied.

The integration of technology with everyday life through the integration of clothing, garments, fashions, accessories or anything worn by a person with one or more computing devices and/or advanced electronic technologies may be provided in various aspects by embodiments described herein. Specifically, the embodiments described herein may provide various garments, such as clothing and accessories, that incorporate computers and advanced electronic technologies. Other wearable technology or devices may also be provided and these are illustrative examples. The term wearable technology extends to anything worn that could have the integration of computers and advanced electronics to improve upon the usefulness of what is being worn. This may extend from clothing that may be worn for fashion to uniforms and clothing meant for work to clothing, body armour and exoskeletons designed for protective purposes or a particular task. These items that an individual can wear will hereinafter be called garments and garments are, but are not limited to, various forms of shirts, hats, vests, coats, gloves, footwear, pants, shorts, and masks; whether they are of light, dense, soft, hard, malleable or rigid fibres, materials or composites. Thus, the integration of technology into any of the above mentioned garments may provide an improvement upon that particular garment if the technology was designed to be used appropriately with it. The foregoing list of garments described is illustrative only and is not intended to be limiting in any way.

Embodiment described herein may incorporate several forms of stimuli (e.g. Sensory Stimulations) and apply them over various distinct fields of practice such as, but not limited to: augmented awareness, entertainment, recreation, training and simulation, medical rehabilitation, and so on. Thus, augmented awareness may refer to any situation where greater awareness of the environment is warranted, needed or wanted, such as providing feedback for the blind to walk and move around reducing tripping and falling and providing GPS directional cues or for the deaf to be alerted to oncoming vehicles, or for a roofer to be warned when they are too close to the edge of the precipice, etcetera. Entertainment includes but is not limited to video games, movies (home televisions or theatre) and music and augmented reality. Recreation includes any activity done for enjoyment when one is not working (massaging, for example). Training/simulation includes but is not limited to the military, police, fire, tactical training and education research. Medical rehabilitation refers to the use of improving the speed at which an individual recovers from particular physiological or psychological problems and physiotherapeutic or psychotherapeutic activities and uses. The types of stimuli include, but are not limited to; electrical stimulation, audio stimulation and the application of forces to the individual.

WPEST allows individuals using a MCEIATR and/or interacting with a virtual medium or other device to receive tissue, nerve and/or muscle stimulation and/or contraction so that the stimulation is precise as determined by its ability to conform to the scientific methodology of repeatability, reproducibility and reliability; this being due to consistency of electrode positioning in one or multiple locals on a wearable garment that correspond to locals on the human body when worn. The wearable garment includes different types of Sensor Devices that actuate to provide different types of Sensory Stimulation. As an example, electrical stimulation (as an example of Sensory Stimulation) provided by electrodes (as an example of Sensory Devices) may be of any form of stimulation including but not limited to EMS, TENS, MC/FSM, IFS, FES, and NMES). The interaction device can be any form of computing device.

The apparatus can also further include Sensory Devices for Individualized Local Sound which is a way for speakers/subwoofers/audio output devices (hereinafter referred to as a speaker) to be implemented to give an individual highly accurate directional sound relative to an individual's placement in regards to a particular application without worrying about the constraints of the physical environment, the individual's physical location in an environment or other individuals in the physical environment.

Additionally, the Sensory Device may include a Force Simulating Device, which is a mechanical componentry within garments to simulate the effects of forces on the body. The componentry is designed to be controlled via a computing device (or Control Centre) that sends data to the Force Simulation Device to determine what forces to apply to the individual. The computing device sends activating signals to actuate the Force Simulation Device to produce Sensory Stimulations as force stimulation. These forces are to give an individual the sensation of motion whether it be a push, pull, twist, tension, compression or constriction applied in a particular direction or the feeling of centripetal or centrifugal force. Through these sensations or physiological stimulations it allows an individual to feel particular forces that may be in effect. The hardware does not need to be associated with a particular medium as it can work with a variety of types of computing devices that have the ability to send data to the device that would activate its mechanical componentry to create one of its various effects. Further to this, a Sensory Device may also include Constriction/Compression Stimulation Device which may have the ability to apply local, general, or total hugging, squeezing, contracting, tightening, or crushing to the individual using embodiments described herein.

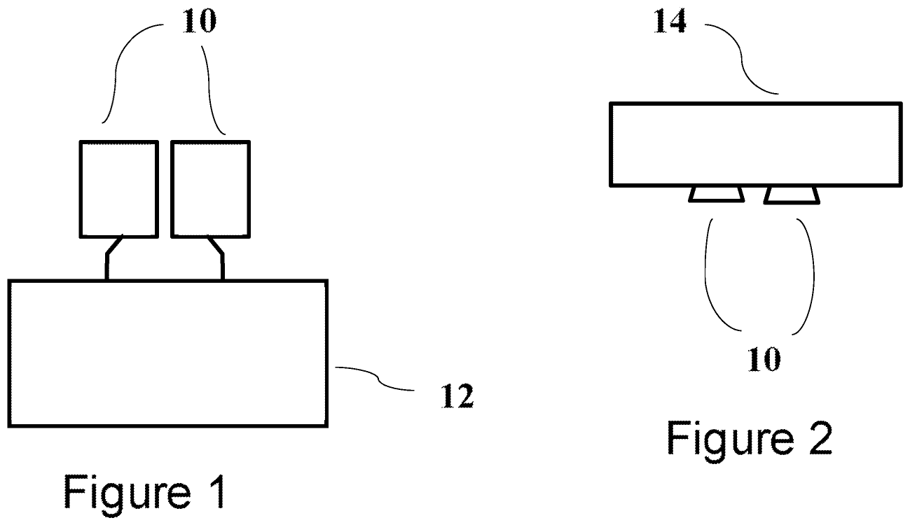

FIG. 1 shows electrodes 10 and an MCEIATR 12 which are operably connected. In the embodiment shown, the two are hardwired together, but they may be coupled using various wired and wireless technologies. FIG. 2 depicts the application of the electrical stimulus interfaces (electrodes) 10 via a wearable material 14 which are predetermined and definite within said garment 14. The electrodes 10 are an example Sensory Device. The electrodes 10 can deliver multiple variations of Sensory Stimulation including, but not limited to: EMS, TENS, MC/FSM, IFS, FES, and NMES. This stimulation and/or the variations thereof also can be applied and/or delivered simultaneously, consecutively or intermittently.

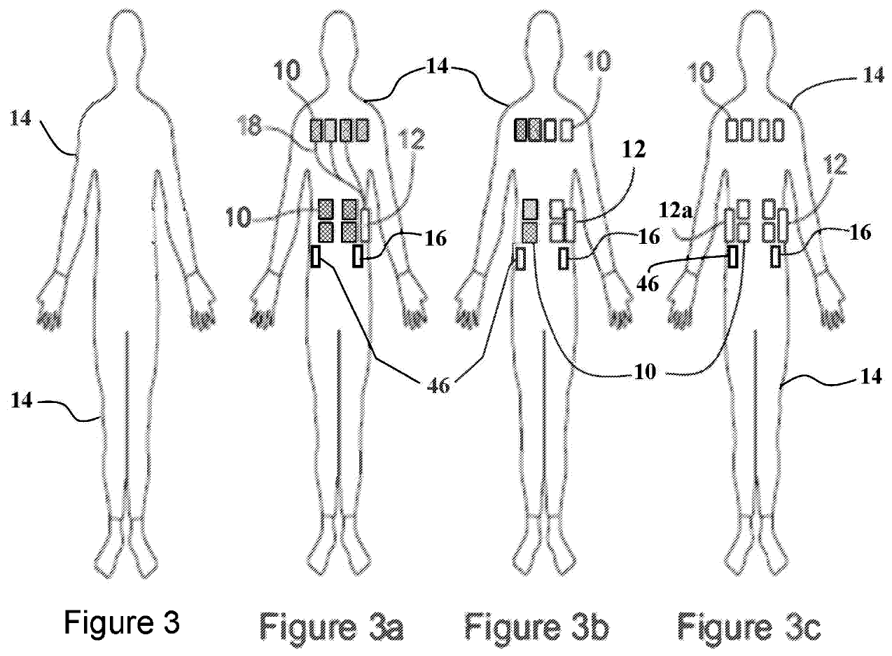

FIG. 3 illustrates wearable material 14. FIGS. 3a, 3b and 3c depict different examples of electrode 10 stimulation on wearable material 14, and how the MCEIATR 12 can select which electrodes 10 are to be stimulated and which are not. A Signal Path of a Sensory Event may define which electrodes are to be stimulated (to produce Sensory Stimulations) and which are not. Electrode placement is for illustrative purposes and may or may not be positioned as shown. FIG. 3a shows all electrodes 10 being actuated via a wired connection 18 to produce Sensory Stimulations. FIG. 3b shows some electrodes 10 being actuated to produce Sensory Stimulations. FIG. 3c shows no electrodes 10 actuated to produce Sensory Stimulations. The embodiments also provide reliability through predetermined and defined electrode 10 placement which may correspond to different physiological locations. As electrode 10 positioning relates to type and the frequency of stimulation (to produce different Sensory Stimulations) for activated Sensory Events and the ensuing physiological response and the sensory perception from this response (Sensory Signature or Sensory Outcome), it is imperative that for the same sensory simulated response (Sensory Signature or Sensory Outcome) to occur and that the process is repeatable, consistently reproducible and reliable. Repetition refers to the ability to repeat the same action or activity with no change in the setup or configuration. In some embodiments, the positions of the electrodes 10 are user adjustable and the wearable material 14 can optionally have indicators detailing the position of electrode 10 to allow an individual to accurately repeat the placement. Alternatively, an embodiment could have a set number of allowable locations for the electrodes 10 within the wearable garment 14. Accordingly, to be repeatable the electrodes 10 may be positioned similarly each time so that they will provide the same Sensory Manipulation or Sensory Stimulation. The electrodes 10 are predetermined and defined and they then maintain this positioning in the garment 14. Predetermined and defined refers to the permanent or non-permanent placement of electrodes 10 on the garment 14 which maintain their position relative the garment 14 unless, in the case of non-permanent, otherwise moved by an individual or removed for replacement due to wear.

The garment 14 houses the Control Centre 16. The Control Centre 16 is a computing device which may contain a Mem Chip, profile selector, transceiver, USB port, actuation module, and sensory software. Control Centre 16 is the signal processor

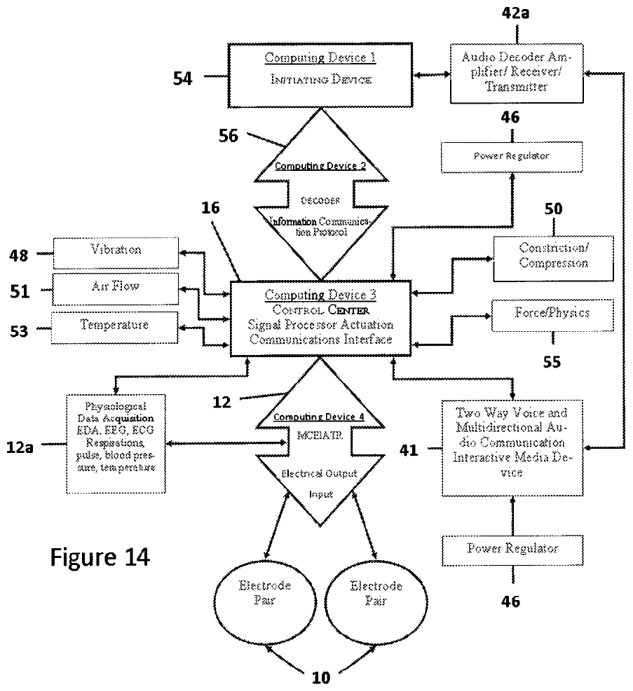

actuation and communications interface 16 (as detailed in FIG. 14). In some embodiments, the Transceiver may be integrated into the garment (Exoskeleton of As Real As It Gets (ARAIG)) while the Mem Chip may be detachable from the Transceiver. For the Control Centre 16 to work it may not require a Mem Chip to be attached to the Transceiver. The Control Centre 16 determines the exact Sensory Manipulation that occurs by defining different Sensory Events as Signal Paths for actuation of the Sensor Devices in different ways. A Signal Path may define different combinations or sets of Sensor Devices to actuate for Sensory Events and parameters of the Signal Path may define different types of Sensory Manipulations produced by the actuated Sensory Devices.

The Transceiver is the component of the Control Centre 16 that activates the necessary Sensory Devices by transmitting activation signals to the Sensory Devices. The Sensory Devices (e.g. electrodes 10) make up the components of ARAIG's Nervous System. This activation is based on the translated sensory data from the Decoder 56 and the stored user settings of the attached Mem Chip. The Control Centre 16 determines Sensory Events using the sensory data and the user settings. The data received from the Decoder 56 is the raw data to determine what Sensory Devices of the Nervous System should be activated and the Sensory Stimulation(s) that they will produce, as stipulated in the determined Sensory Events. The settings taken from the Mem Chip allow the Transceiver to alter the raw data from the Decoder 56 to select Sensory Events that activate to actuate Sensory Devices to produce Sensory Stimulation(s) that are within acceptable ranges of the Mem Chip's Personalized Settings. Therefore, the Transceiver receives the data from the Decoder 56, alters the data as required by the Mem Chip's Personalized Settings and then activates the appropriate Nervous System component(s) Sensory Device(s), to provide the correct Sensory Signature(s) or Sensory Stimulations for the Sensory Events. If there is no Mem Chip attached to the Control Centre 16 then the Transceiver may use the raw data from the Decoder 56 to activate the Nervous System.

The Mem Chip is the component of the Control Centre 16 that stores the user's Personalized Settings to determine the maximum and minimum sensations of the Nervous System's components. The Personalized Settings may also define one or more Sensory Events which may be customized for a user or situation. The default setting of the Mem Chip may allow all the components, of the Nervous System to be activated to maximum capabilities. To alter the default setting of the Mem Chip the user may run ARAIG's Calibration and Diagnostics Protocol (e.g. video game). With the Mem Chip settings updated and stored for any further use the components of the Nervous System may now be set to the Personalized Settings of the user rather than the default settings. In one embodiment, if they use the Calibration and Diagnostics Protocol more than once, the “final use” may create another profile on the Mem Chip and will set this as the new active Personal Settings. In another embodiment the user may choose to store this “final use” profile as a secondary or other profile in their number of saved user profiles.

Since the Mem Chip may not deal directly with the data sent from a wearable device and only alters the translated data, the Mem Chip's Personalized Settings may be universal for all devices. This allows a user the ability of setting their Personalized Settings on one wearable device and using them on any wearable device. This ensures that the user only has to update their Personalized Settings when they want something changed and not when something has changed on them.

The design of the Mem Chip may be that of a USB or other type of device to be detachable so that it can be attached to a wearable device or another ARAIG. The purpose for attaching directly to another device is to update the Mem Chip should any alterations, patches, or mods be required or wanted and the ability to store their Personalized Settings externally. The use of externally storing their Personalized Settings also allows for the user to share their Personalized Settings with others and have multiple Personalized Settings at their disposal. As for attaching to a different ARAIG, this protocol may allow an easy transfer of all of their preferences without having to go through any previous setup. This transfer of one Mem Chip to another ARAIG will be possible for ARAIG's of the same generation but it may not be possible for different generations as they may be more complex and their Mem Chip software could be different to match the changes. Meaning, upon purchase of a new ARAIG of a different generation they may need to go through a Calibration and Diagnostics Protocol for that generation of ARAIG.

The Nervous System may be the portion of ARAIG that contains the immersive qualities, the Sensory Events defining different Sensory Stimulations and Sensory Manipulations, as well as the Physiological Data Acquisition capabilities and may be the interactive portion of the product. The Nervous System may be attached to the Exoskeleton and its sensory components (e.g. Sensory Devices) may be activated by the Control Centre 16 through activation signals. The activations of specific Sensory Devices of the Nervous System may translate into tangible Sensory Stimulation to the user.

The Control Centre 16 is the device that will actuate the MCEIATR 12 which in turn provides the stimulus through the electrodes 10 or other Sensory Devices. The garment 14 is applied or fitted into position onto the user. The electrodes 10 may be already predetermined to affix to the skin of the user in the desired anatomic location in some embodiments. In some embodiments the electrodes may be prepositioned and permanently affixed within the garment. Each time the garment 14 is fitted onto the user the configuration of electrodes 10 may remain fixed, unless changed by the user, thereby stimulating the exact anatomical elements as previous or providing the same Sensory Stimulations as previous. This repetition may be performed until such time as the electrodes 10 need to be replaced. The new electrodes 10 may take the exact same position on the garment 14 as those being replaced thus allowing for the unlimited repetition of this activity which allows for consistency in the reproduction of the desired Sensory Signature or Sensory Event over a period of time. Reproducibility is the ability of the entire process or action to be reproduced, either by the user, producer, director, initiator, or by someone else working independently. The embodiments described herein provide this reproducibility and subsequent reliability. The embodiments described herein provides a new and inventive methodology, system, and architecture for the provision of this repetition, reproducibility, and reliability which makes the outcomes precise as desired by the user, producer, director and/or initiator of the prescribed stimulus for many applications.

A minimum of two electrodes 10 may be used for some embodiments, but in other embodiments, an array of electrodes 10 may be attached to the garment 14 to give accurate localized stimulation.

WPEST has the ability to provide and produce more than one type of stimulation which may include but is not be limited to EMS, TENS, MC/FSM, IFS, FES, and NMES. A Sensory Event may define different types of Sensory Stimulation to produce different Sensory Outcomes. These varying Sensory Stimulations may occur singularly or in any combination: synchronous, intermittent, consecutive, imbricate, etc. The pattern and configuration for the Sensory Stimulations may be defined by a Signal Path of a Sensory Event to produce desired Sensory Outcomes.

This singular or multiple stimulation(s) may occur on or over one or more sets of electrodes 10. The Sensory Event may define different sets of Sensory Devices for actuation at activation of the Sensory Event. For example, a wearer can receive TENS applications to the shoulder while simultaneously receiving EMS applications to the lower back.

Embodiments described herein may also provide and produce more than one type of Sensory Stimulation on the same plurality of electrodes 10. For example, a wearer can receive an FES application to their right shoulder which is consecutively followed by a TENS application through the same electrodes 10 to that same right shoulder.

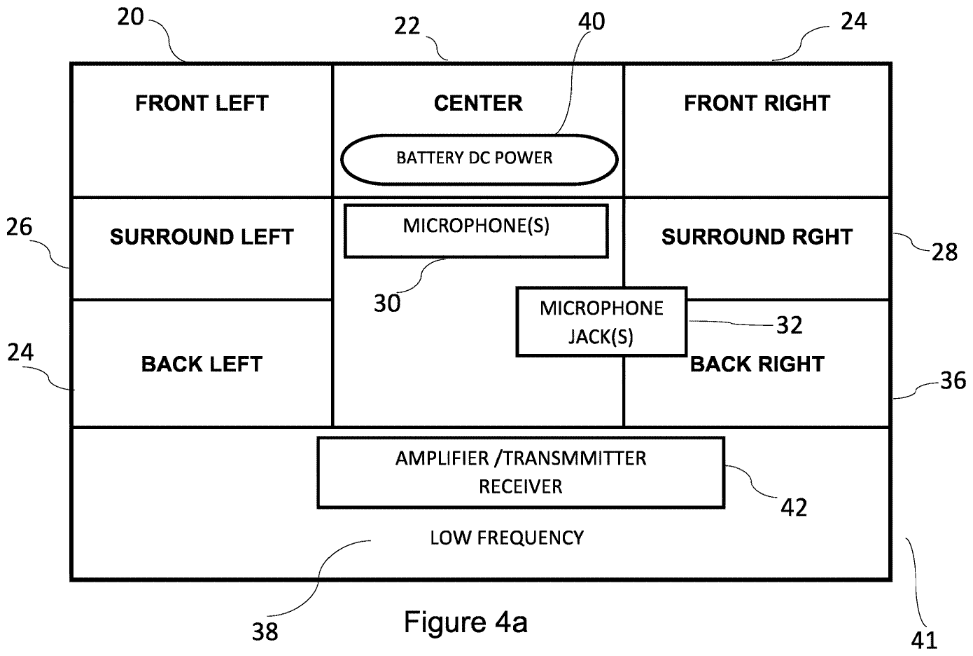

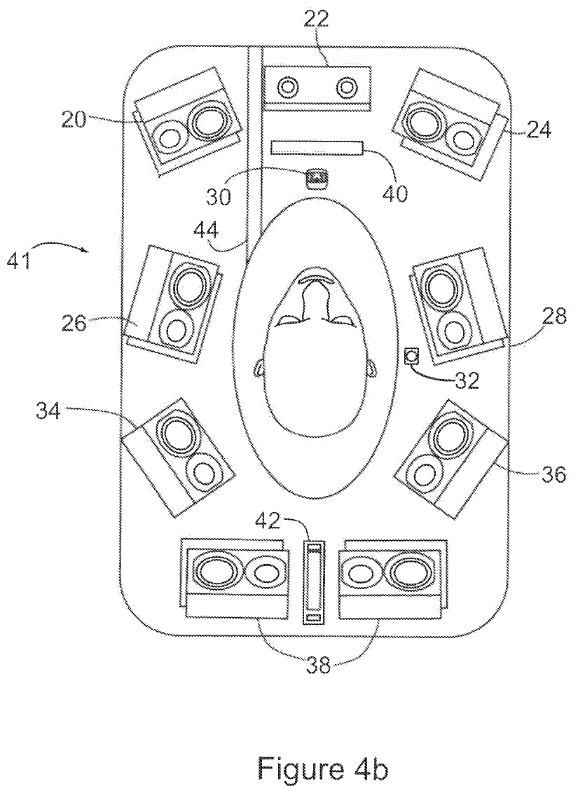

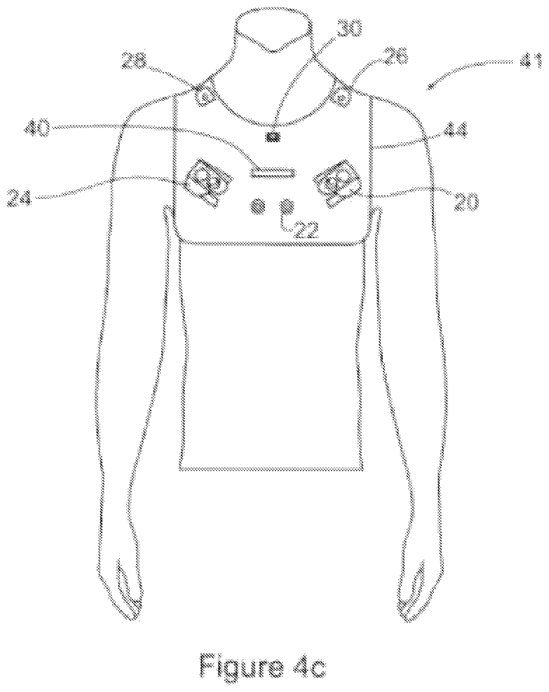

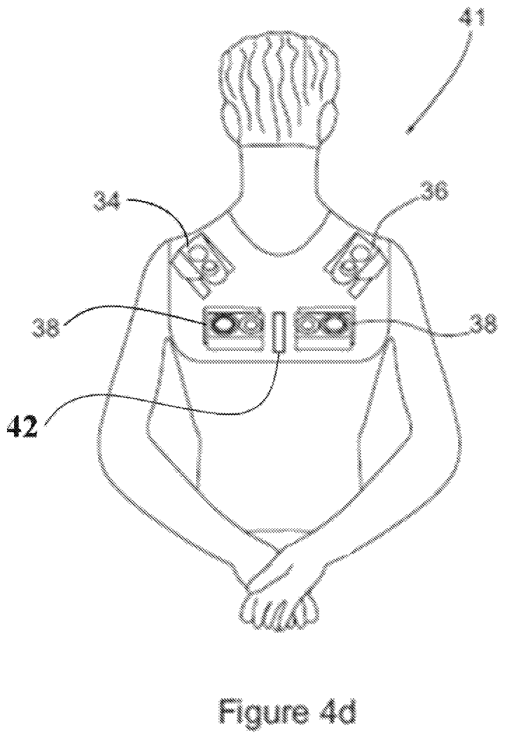

FIGS. 4a, 4b, 4c and 4d show the incorporation of speakers into the garment 14 as another example of Sensory Devices that produce audio physiological stimulations as Sensory Stimulations. As depicted, the embodiment of individualized surround sound 41 has front left speaker 20, center speaker 22, front right speaker 24, surround left speaker 26, surround right speaker 28, back left speaker 34, back right speaker 36 and low frequency subwoofer speakers 38. Additionally, the suit allows the user audio input via a microphone 30 or the use of an external microphone which can be plugged into microphone jack 32. The device is powered by its power source 40 which may be secondary to the power source provided to the Control Center 16 and myriad Sensory Devices. The power source can be anything that effectively supplies power to the device, including, but not limited to; batteries, a cable plugged into an outlet, or another device that supplies AC, DC or a combination thereof. FIG. 4b additionally depicts an amplifier/transmitter/receiver 42 that is operatively connected to the initiating device or an Audio Decoder 42a and receives, amplifies and transmits the incoming data to the speakers. The embodiment shown in FIG. 4b also has an opening area 44 which can be opened and closed for donning and removing the device. Possible non-permanent closing methods include; hook and loop type fasteners, zippers, snaps, buttons, and etcetera.

In another embodiment a second amplifier/transmitter/receiver the Audio Decoder 42a as shown in FIG. 14 may be utilized which takes the information directly from the initiating device 54 and transmits it to the amplifier/transmitter/receiver 42 affixed to the garment 14. The communications and wireless communications protocol for the Audio Decoder may be one or more of, but is in no way limited to high definition multimedia interface (HDMI™), wireless fidelity (Wi-Fi), Radio, Bluetooth™.

The use of the Individualized Local Sound can be used with any form of computing device. Individualized Local Sound can be used for computing devices implementing virtual mediums and many real world situations. The efficiency of the sound system designed in this manner is that the speakers maintain their position around the wearer; ensuring that the wearer is always in the optimal position, “sweet spot”, for surround sound. Unlike traditional sound systems which are placed at particular locations in an environment or headphones which are placed to rest on the head and have the speakers located on or in the outer portion of the ear, Individualized Local Sound is a form of Wearable Technology. Individualized Local Sound is the integration of speakers into the garment 14 worn by the individual at different positions. In another embodiment, the most relevant to the auditory system are but are not limited to those that cover the torso, upper arms and head as these are located near the ears and would have the least amount of change in location in relation to the ear; while the lower arms and legs which could be in motion or at various angles that would make speaker placement much more difficult. Therefore it may be easier to integrate speakers in the previously mentioned locations and would be the major area of interest for Individualized Local Sound.

By integrating a single speaker into a garment 14 it allows the speaker to be placed in a particular location that will remain located in the same position relative to the individual using the speaker. This allows for a single speaker to represent a particular direction sounds are emanating from while still having all of the original functionality that a speaker would permit such as volume, bass, and son, operably connected to a computing device. In addition, Individualized Local Sound can then be extended by integrating multiple speakers into a garment 14. By integrating more speakers it may allow an individual to receive sound that has accurate multidirectional feedback, is relative to their location and individualized to them rather than designed for the environment. The Control Centre may implement sound stimulation by selectively choosing volume levels in individual speakers of the sound system. This may allow for example a car to actually sound like it is moving passed the individual, or when someone is talking to the individual this other individual could be heard via the speakers that represent the direction they are located; will increase the auditory awareness, reaction time and overall immersive experience.

In some embodiments, the material that provides the housing for the sound system may be of a sound absorbing nature. The speakers themselves may be angled in such a way as to provide the auditory cone (sound cones) to be directed to affect the best possible auditory experience and/orient the sound at the user. This means a plurality of users may use the individualized surround sound and minimally disrupt each other.

The usefulness of added speakers in the manner described is that individual may have a much more accurate sound experience which in turn may improve the individual's auditory experience. It may be an improved experience because the individual will now be better aware of the direction to which sounds are emanating from. Such an example would be an individual playing a game or a simulation whereby the individual is represented by a particular avatar in the virtual medium and wherever they are located the location of the sounds would be created relative to their location in the virtual medium; thus allowing for an accurate representation and greater level of awareness of their surroundings in this virtual medium. Furthermore, through this design each individual may be able to have the same sound experience as any other individual wearing the embodiment shown herein without having to worry about others or the environment, such as in a theatre with multiple people or at an individual's house with a surround sound system. Thus, the issue of a sweet spot (whereby a sound system only has a particular region that the sound is heard at the quality it is expected and outside that region it is not) may be eliminated because each individual is now located in their own sweet spot due to Individualized Local Sound. This may ensure whether you have multiple people in the same room, theatre, just one individual in one room or an individual moving from room to room the auditory experience remains the same. Furthermore, it may allow individuals to wear garments 14 that are expected to be worn for their particular task (i.e. training, research) rather than wearing or using hardware that would not realistically be part of the experience. Such a situation where this may be beneficial is training. Whether it be military, police, fire, etc. this may allow individuals to wear the same garments they would in the actual real-life situation rather than wearing particular equipment or having the equipment built into the environment, which could potentially alter the training experience and its benefits to real world scenarios. This may be important because it may allow individuals to be trained more realistically and may not matter how the environment is designed. Another major benefit of this design may be mobility; most current high end sound systems do not allow for great mobility. The ability to be mobile allows an individual to have the same experience on the go or in any particular environment with fewer adjustments to the Individualized Local Sound unlike other elaborate sound systems. In comparison to other mobile sound systems such as headphones, Individualized Local Sound allows for a more accurate localization of sound and can allow for an increased number of output locations for the sound to better represent what is occurring with a particular application (i.e. thus a greater auditory experience. Overall, the creation of Individualized Local Sound allows for a more accurate, realistic, and personal sound experience that is unaffected by the individuals environment and therefore enhances the overall experience of any sound related application. As shown in FIGS. 4b, 4c and 4d, embodiment include a plurality of speakers 41 positioned to surround the head of the user on the shoulders of the garment 14, the upper chest and upper back, with an additional subwoofer 38 located on the back of the user. FIG. 4c depicts the front of the user while FIG. 4d depicts the back of the user. Embodiments may effectively create an individualized surround sound experience for the individual wearing it. The parts of the body chosen move little relative to the individual's ears; creating a mobile and consistent sound that is as dynamic as the individual.

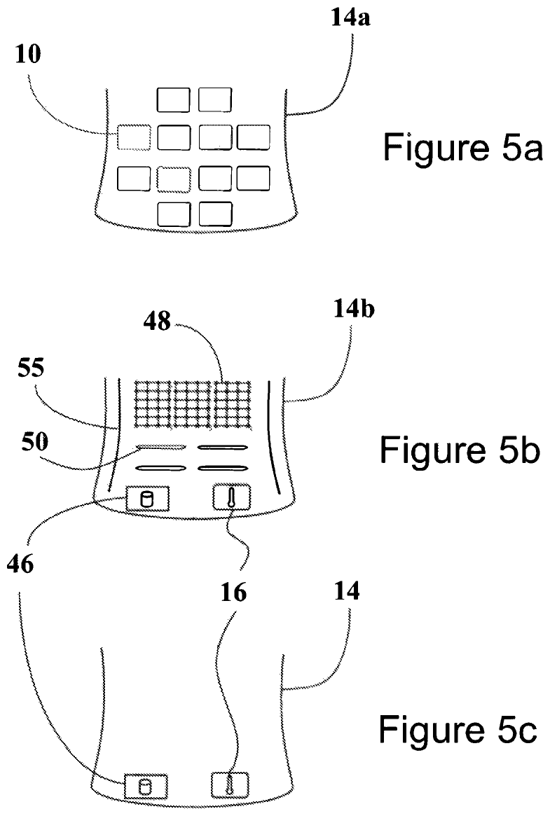

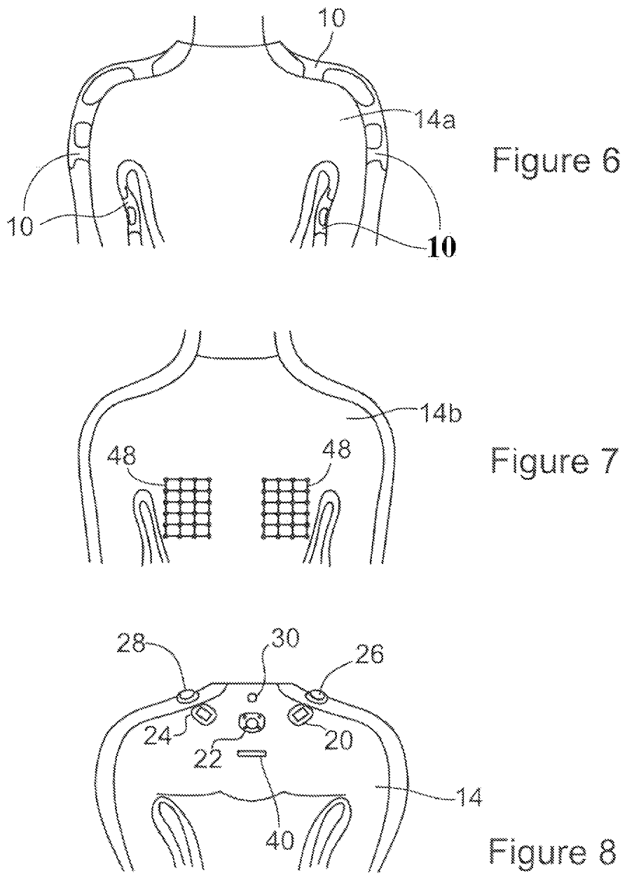

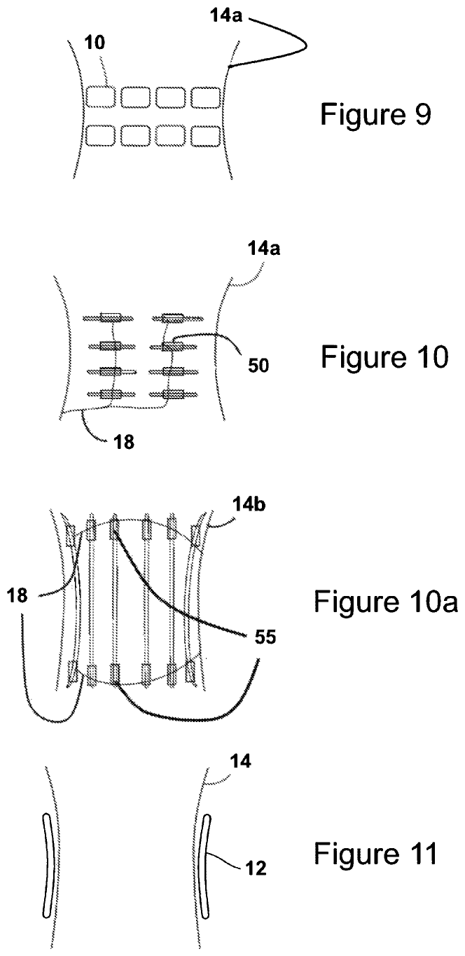

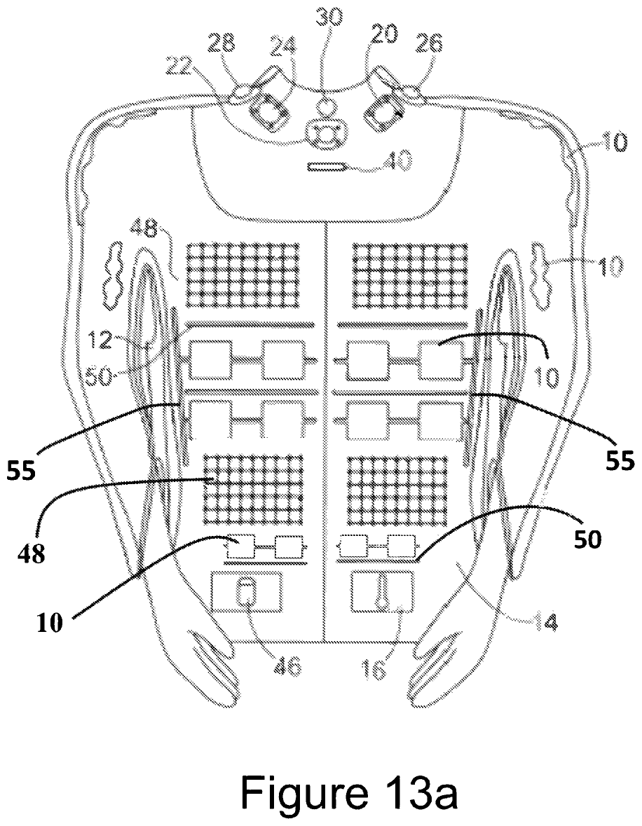

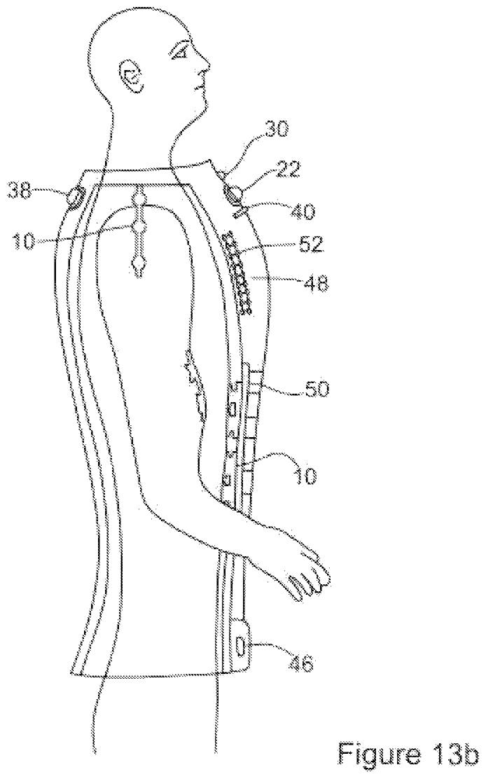

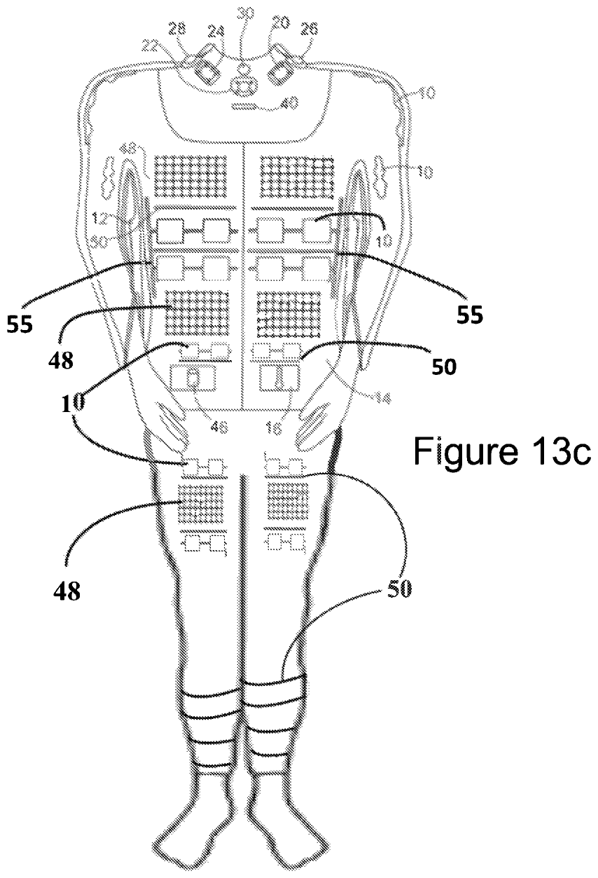

To represent the configuration of the Sensory Devices and componentry within a garment 14 the frontal view of one embodiment shown in FIG. 13c is separated into three garment areas of the body; one being the abdominal area (shown in FIGS. 5a, 5b, 5c, 5d); one being the upper torso or chest and shoulder area (shown in FIGS. 6, 7, 8); and one representing coverage of both the abdominal and torso area (shown in FIGS. 9, 10, 10a, 11). All components may be interconnected and representative of and make up an embodiment (e.g. as shown in FIG. 13c) whereby the synergy and totality of Sensory Manipulation and the Sensory Stimulations defined thereby implemented throughout the entire garment as defined by the Electronic Signal Pathway of the Sensory Event (e.g. as shown in FIG. 14) creates the Sensory Signatures which produce the desired Sensory Outcomes. This total body garment and experience is an example, and individual portions or combinations thereof may be used in other embodiments.

FIGS. 5a, 5b, and 5c show a frontal view of the abdominal area of an embodiment and, separate this view to show the different layers of the garment 14 and the components attached or affixed to or in each. This is to show the different components that exist but does not include any overlap as this would obscure from view those things underneath. Therefore, the componentry configuration shown is designed as a representation of the embodiment but the actual design will contain a greater number of sensory stimulation components than shown here.

FIG. 5a shows the inside first layer 14a which would contact the user or the users underlying clothing. Electrodes 10 are shown in FIG. 5a attached or affixed to the inner portion of the first layer 14a.

As shown in FIG. 5b, vibration actuators 48 and Force Simulation Device/Constriction/Compression Stimulation Device actuators 50 are depicted attached or affixed to the interior or the exterior of the second layer of the garment 14b. FIGS. 5b and 5c shows the Control Centre 16 and power regulator 46. FIG. 5b shows the Control Centre 16 and power regulator 46 attached to the exterior of the second layer 14b. FIG. 5c shows that the Control Centre 16 and power regulator 46 may even protrude through the third layer 14, giving the user access.

FIGS. 6, 7 and 8 show a frontal view of the upper torso and chest area of an embodiment and, separate this view to show the different layers of the garment 14 and the components attached or affixed to or in each. This is to show the different components that exist but does not include any overlap as this may obscure from view those things underneath. Therefore, the componentry configuration shown may be designed as a representation of the embodiment but the actual design will contain a greater number of sensory stimulation components than shown here.

In the embodiment shown in FIG. 6, electrodes 10 may be attached to the inner portion of the first layer of the garment 14a. Vibration actuators 48 (another example Sensor Device) which may be located in predetermined locations to either the exterior of the first layer 14a or interior of the second layer 14b as shown in FIG. 5b and FIG. 7. Similarly, the speakers 20, 22, 24, 26, 28, subwoofer speakers 38 (not shown), microphone 30, microphone jack 32 (not shown) and power source 40 of the surround sound system each may be located in a defined and predetermined location on the interior or exterior of the third layer of the garment 14 as shown in FIG. 8. The components of the surround sound system may have a similar layout as shown earlier in FIGS. 4a, 4b, 4c and 4d. Additional electrodes 10 may be included in addition to those shown in FIG. 5a, FIG. 6 and FIG. 9. These additional electrodes 10 may be attached to the inside layer of the garment 14a in defined and predetermined locations.

FIG. 10 shows Constriction/Compression Stimulation Device actuators 50 in defined and predetermined locations and attached to the exterior of the first layer of the garment 14a.

FIG. 10a shows Force Stimulation Device actuators 55 in defined and predetermined locations and attached to the interior of the second layer of the garment 14b.

As depicted in the embodiment of FIG. 10, the actuators 50 run in a grid-like fashion, top to bottom seemingly overlapping one another. FIG. 10 shows one group of actuators 50 that encircles the body. The actuators may encircle the body running in parallel across the body horizontally i.e. left to right or right to left where the actuators are located on the exterior of the first layer 14a.

FIG. 10a shows one group of actuators 55 that runs up and down the body in parallel with each other. The embodiment exhibits where the actuators may run vertically i.e. top to bottom on the interior or exterior of the second layer of the garment 14b. FIG. 11 shows an embodiment where the MCEIATR 12 is in a defined and predetermined location(s) and attached to the exterior layer of the garment 14.

FIG. 10 and FIG. 10a shows the position of the actuators 50, 55 of the Constriction/Compression Stimulation Device/Force Simulating Device. The Force Simulation Device may apply physical forces to an individual so that they feel particular sensations that would normally pertain to a particular real-world event. Such sensations could be but not limited to imitating the centrifugal force that is felt as an individual driving turns a corner, someone pushes or bumps into an individual, or the weight of carrying something on one's shoulders, and so on. Other forces may also be simulated. The Force Simulation Device may be able to directly apply these forces to specific locations of the body as it is a form of wearable technology. Thus, the Force Simulation Device may be integrated into the garment 14.

The Force Simulation Device (actuator 55) allows for localized forces to be applied to an individual. Through the use of a computing device it allows a Force Simulation Device to alter such parameters as the amount of force that is applied (minimal to maximum), the speed at which the force reaches its target amount (fast or slow), the duration to which the force is applied (amount of seconds or deactivates one target force is reached) and the speed at which the force is removed (fast or slow). Through these different parameters it allows for a multitude of forces to be simulated at a given location within the garment 14 an individual is wearing. In addition, by extending the Constriction/Compression Simulation Device 50, Constriction/Compression Stimulation Device actuator 50, and Force Simulation Device 55, Force/Physics Stimulation Device actuator 55, to cover multiple regions of a garment or garments 14 which in turn covers a larger region of the individual (as shown in FIG. 13c) which increases the amount of forces that can be applied simultaneously and the ability for an individual to more accurately determine the direction of the force as well as what the force might represent for a particular application. Such sensations that could be created are that of a strong or weak, gradual or quick, constant or instantaneous simulated force in one or multiple locations simultaneously.

Force Simulation Device is useful as it allows for virtual mediums to have an increased immersive experience as a force applied to the body will give the intensity of the force applied and the direction to which the force came from based on its location in the garment 14. In addition, the use of a Force Simulation Device for simulations and training creates additional forces for the particular application giving a more realistic experience. This increase in realism better prepares individuals for the real-world experience to which the simulations and training are designed for.

For example, in a simulation, the individual is moving backwards and encounters an obstruction; the individual may immediately feel the height of the object and can determine, without turning around, whether it is possible to climb or jump over or whether to find another route.

In one embodiment, shown in FIG. 10, linear actuators are attached to the exterior of the first layer of the garment 14a. FIG. 10 depicts potential Constriction/Compression Stimulation Devices 50 being actuated via a wired connection 18 as determined by the Control Centre 16 (not shown). In this illustrative example, the actuators draw in their attached 1″nylon webbing which shortens the length thereby reducing the diameter of the webbing which circumnavigates the user and performs a constricting action.

In another embodiment, FIG. 10a depicts a potential implementation of Force/Physics Stimulation Device 55; linear actuators are attached to the interior or exterior of the second layer of the garment 14b. They may be actuated via a wired connection 18 as determined by the Control Centre 16 (not shown). The shortening of the vertical webbing may cause a pulling action which draws down on the users torso as if gravity is affecting the user.

The Constriction/Compression Stimulation Device 50 and Force/Physics Stimulation Device 55 may be used with any computing device to create the effects. The computing devices may be but is not limited to using the Constriction/Compression Stimulation Device and Force/Physics Stimulation Device 55 to sync the sensations with a virtual medium or in use in real world applications. The Constriction/Compression Stimulation Device allows computing devices to add to their applications the capabilities of applying a compression and/or constrictive feeling to a location of an individual's body. This sensation may also be described as tightening, pressure, crushing, squeezing, and contracting. To properly compress or constrict a part of an individual's body the Constriction/Compression Stimulation Device is a form of wearable technology. The Constriction/Compression Stimulation Device is integrated into a garment 14.

Through the use of a computing device the Constriction/Compression Stimulation Device can have various parameters altered to effect the sensation of constriction/compression and squeezing such as but not limited to the pressure (minimal or a lot), tightening (minimal or a lot), speed that squeezing or constriction/compression occurs or is removed (fast or slow), the length the constriction/compression is activated for (multiple seconds or once fully activated revert to deactivated state) and the ability to fluctuate between these settings while already activated. Furthermore, since the Constriction/Compression Stimulation Device is wearable technology it may allow for accurate constriction/compression as it will be directly against the individual's body and localized to a particular part of the individuals body. In addition, this Sensory Manipulation or Sensory Stimulation can be extended by having multiple regions rather than just one that can be activated to simultaneously squeeze, contract, crush or constrict an individual's body. Such Sensory Manipulation or Sensory Stimulation could be used in a virtual medium to provide but is not limited to, the sensation of something having a hold of the individual such as a hand having a tight grip on the persons shoulder, something wrapped around the individual that is squeezing tightly and maintaining the amount of pressure the individual feels or representing an object falling on an individual and pinning them whereby the pressure continues to get more and more intense. As for the medical rehabilitation industry, this could have implications in that an individual could be using wearable technology with the Constriction Simulation Device to effectively squeeze and constrict particular areas of their body to help them recover while the individual focuses on other activities. Overall, this Sensory Stimulation provides an individual with the particular sensation of one or more locations feeling pressure or constriction/compression of varying degrees.

The Constriction/Compression Stimulation Device can use any technology that can selectively and controllably restrict areas of the garment 14. The actuators of the device may be one or more of, but is in no way limited to: polymeric artificial muscles, liquid filled bladder(s), piezoelectric actuators, electronic polymeric actuators, Carbon Nanotube Artificial Muscles, linear actuators, winding or tensing elements or other systems.

Polymeric artificial muscles, electronic polymeric actuators (EPAs) and carbon nanotube artificial muscles are materials that expand or contract, lengthen or shorten when energy is passed through them. The lengthening and shortening provides the ability to pull and push as well as decrease or increase the circumference of its measurement while encircled around something. Winding and tensing elements like linear actuators can be electronic DC activated devices. Unlike EPAs they are only the actuator and must be connected to something that they can move. The item they attach to (webbing, strapping, and cable, and so on) may lengthen or shorten as the anchored actuator operates. They may also have the ability to pull and push as well as decrease or increase the circumference of the measurement its strapping is encircled around. Further usefulness of the Constriction/Compression Stimulation Device is due to its positioning as a wearable technology; it can accurately affect the same region on an individual's body with Sensory Stimulations reproducing the Sensory Event and repeatedly providing the desired Sensory Signature or Sensory Outcome. Furthermore, the ability to apply this specific Sensory Manipulation through any part of the garment 14 allows multiple regions to be affected simultaneously and with different effects allowing for a multitude of Sensory Stimulation rather than general compression and constriction. In regard to virtual mediums this can allow them to implement new combinations of Sensory Stimulations to provide a more immersive experience. While for real world scenarios this could provide the particular sensation of pressure that otherwise could not be replicated.

Further Sensory Stimulation and Sensory Manipulation whereby a person's physiology is stimulated to sense various, intended and specific sensual outcomes which are associated with the real world but are only being replicated is actuated through vibration technology 48 (as shown in FIG. 5b for example). The actuators of the Sensory Device may be one or more of, but is in no way limited to: electronic or pneumatic or hydraulic actuators, electronic polymeric actuators, linear actuators, brush coin actuators, piezo-electric actuators, vibration motors, tactile transducers, ultrasonic pads, and or mass actuators. This Sensory Stimulation and Manipulation can optionally be incorporated in a manner that the computing device selectively identifies which areas of the garment 14 are to be activated; whether that is the entirety of the garment 14 or individual areas such as upper back or right arm etc. This may be done to imitate sensations such as disorientation, direct impact, skin crawling or when the individual's avatar is in a plane or automobile, and so on.

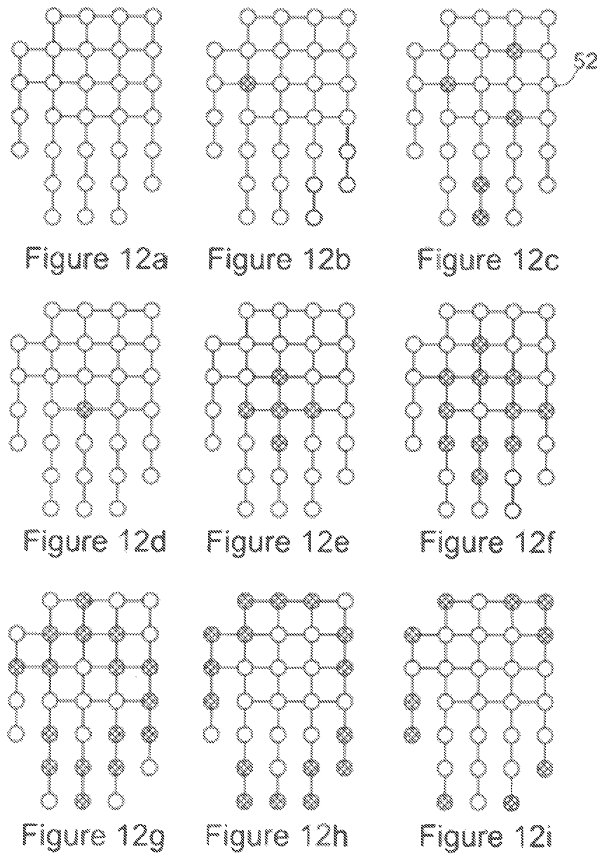

As it is beneficial in the creation of Sensory Events or Sensory Signatures, and ensuing Sensory Outcomes to provide the greatest number of Sensory Stimulations to the user, FIG. 12a through 12i represents some of the actuation and dispersal pattern possibilities of the different combinations of Sensory Device activations. These are illustrative and non-limiting example patterns. There may be singular coverage, multiple coverage, regional coverage or total coverage and may be in arrays, Sensory Event Array(s). The actuators for the force, constriction/compression, vibration and electrical stimulation (e.g. electrodes) 52 in FIG. 12c are preferably situated in the garment so that any activation(s) may form in arrays in the garment 14 as shown in all of FIG. 12. Each dot as shown in all of FIG. 12 may be representative of one or more of these Sensory Device actuations. The different Sensory Events making up the Sensory Event Array may be activated simultaneously, sequentially or through a combination of both simultaneous and sequential activation to produce Sensory Stimulations. This may allow for any computing device to effectively have precision haptics allowing for single location Sensory Stimulation on a user via a Sensory Event or single and multiple location stimulations on a user via a Sensory Event Array culminating in one or more Sensory Signatures for the user.

Sensory related data received from a computing device may create a multitude of stimulations at the position it is located on an individual's body. The location or selection of a Sensory Event may depend on the output of the computing device. The Sensory Event may define different areas or locations of Sensory Devices to actuate to produce Sensory Stimulations. Through the computing device a Sensory Event can be activated to create one or more particular Sensory Stimulation(s) or Sensory Signature, at its location. Through the implementation of multiple Sensory Events creating a Sensory Event Array, the sensory related data received could have a single Sensory Event activated or multiple Sensory Events activated. Furthermore, during the activation of one or more Sensory Events other Sensory Events could be activated and the already activated Sensory Events can be updated to deactivate them or alter the Sensory Stimulation or Sensory Signature, they are creating. This allows the creation of a single or multiple Sensory Stimulation effects simultaneously, sequentially or intermittently in one or more locations on an individual's body for Sensory Events. The placement of a Sensory Event may determine what part of an individual's body feels the stimulation while a Sensory Event Array of FIGS. 12a-12i may be localized to a particular part of an individual's body such as the hand, it could cover the entire arm or it could even cover the entire body depending on its use. Also, depending on the amount of precision required an Sensory Event Array can be created so that the proximity of the Sensory Events in the Sensory Event Array of FIGS. 12a-12i are closer or further apart and the location is entirely dependent on the application and uses for the technology; such that simulations and medical applications may require exact precision while entertainment may be feasible with approximate locations.

In one embodiment, the wearable device is able to create accurate precision Sensory Stimulation as well as unique Sensory Stimulations dependent on the sensory related data received to activate a Sensory Event. The sensory related data is not limited to affecting the stimulation's duration, intensity and radius, as shown in FIGS. 12a-12i. To further increase the effectiveness of a Sensory Event, many Sensory Stimulations can be combined together to create a multitude of stimulation effects and creates a Sensory Event Array (e.g. FIGS. 12a-12i). In this regard, Sensory Stimulations can be created that covers a much larger area but retains the ability to be just as precise from one Sensory Event to the next. Thus the Sensory Stimulations may still replicate stimulation at a single locale (FIG. 12b) or at multiple locals simultaneously (FIG. 12c), sequentially or a combination of the two. The Sensory Signature(s) or Sensory Events and ensuing Sensory Outcome(s) that may be created is not limited to Sensory Manipulation or Sensory Stimulation of a single Sensory Device or a singular Sensory Event or a singular Sensory Event Array. Nor is it limited to the Sensory Manipulation of multiple Sensory Devices or multiple Sensory Events or multiple Sensory Event Arrays. It may initiate as one or the other and expand or contract to provide more or less Sensory Stimulation to the user in some embodiments. Therefore, Sensory Signatures or Sensory Events and ensuing Sensory Outcomes may contain multiple Sensory Stimulations activated to represent stimulations from a single source to multiple stimulations activated to represent Sensory Stimulations from multiple sources to a combination of single and multiple Sensory Stimulations from one or more sources. In the context of a video game, there are various uses for this technology: such as, although not limited to, the activation of one or more Sensory Events to create the specific Sensory Signature (or combination of Sensory Stimulations) such as the effect of bullets impacting or shrapnel of a grenade or rocket impacting off armour; a slice from a knife or sword whereby multiple Sensory Events are activated sequentially one after another affecting different Sensory Devices 52 of the Sensory Event Array of FIGS. 12a-12i in a line or particular pattern. For example in FIGS. 12d to 12i a ripple effect starting at a particular location on the Sensory Event and working outwards much like a concussion effect; a wave effect whereby it starts at one or multiple locals to create a stimulation across a line that moves from a particular part of an Sensory Event Array to another part of the Sensory Event Array; a stimulation completely across the Sensory Event Array much like something being scanned; or trying to scare an individual in a haunted house by creating the feeling of something moving across an individual's back but visually there is nothing there. Overall, whatever the desired Sensory Outcome of a single Sensory Event or multiple Sensory Events in the form of a Sensory Event Array, the number and layout of the Sensory Events is entirely dependent on the Sensory Outcome defined by the Sensory Manipulation. Embodiments may implement use of a single Sensory Event to be able to create precision Sensory Signatures (or combination of Sensory Stimulations) for the user and have an ability to be expanded upon to create such a variety of Sensory Outcomes.

In another embodiment Sensory Events may produce Sensory Signatures (or combination of Sensory Stimulations) and Sensory Outcomes through Sensory Device activations to certain coverage areas of the body: singular coverage; multiple coverage; regional coverage; total coverage; and dispersal coverage. A singular coverage area may include just one area of the body. It may be a specified area of small coverage and the Sensory Event may include the actuation of one or more Sensory Devices within the Nervous System to create Sensory Stimulations. For example, singular coverage may include Sensory Device activation(s) in the proximal portion of the arm (humorous, biceps, triceps, and upper-arm). The singular coverage areas actuate as determined by the Control Centre activation signals. Multiple coverage areas include two or more singular coverage areas of the body. They may be adjacent body areas or detached from one another. This Sensory Event may be made up of specified areas of coverage and may include the actuation of one or more Sensory Devices within the Nervous System. For example, multiple coverage areas may include the proximal portion of the arm (humorous, biceps, triceps, and upper-arm) and the connecting deltoid/shoulder. Or multiple coverage areas may include the proximal portion of the right arm (humorous, biceps, triceps, and upper-arm), the left medial pectoral/chest and the right lateral portion of the abdomen. The multiple coverage areas will actuate as determined by the Control Centre activation signals. Regional coverage area includes adjacent quadrants or sections of the body. A Sensory Event to these specified areas of coverage may include the actuation of one or more Sensory Devices within the Nervous System. For example, regional coverage may include the thoracic and abdominal cavity both medial and lateral. Or regional coverage may include the proximal and distal portion of the left arm, the adjacent left shoulder, chest and abdominal areas. The regional coverage areas will actuate as determined by the Control Centre activation signals. Total coverage area includes all coverage areas of the body. The Sensory Event for this specified coverage may include the actuation of one or more Sensory Devices within the Nervous System to produce Sensory Stimulations. For example, all areas of coverage would provide Sensory Stimulation to the user: arms, legs, and torso. The total coverage areas may actuate as determined by the Control Centre activation signals. Dispersal coverage is similar to the Sensory Event Array and includes one, two or more singular coverage areas of the body. When more than one singular coverage area is involved they are adjacent body areas. The Sensory Event initiates in one specific point in the singular coverage area and radiates, moves, flows, ebbs, surges outward, inward, up and down, etcetera to the end of this singular coverage area and then continues flowing where necessary through other coverage areas as directed by the Control Centre activation signals. For example, the Sensory Event starts in the distal portion of the right lower arm and pulses in a wave like fashion up through the proximal portion of the arm into the right shoulder and down into the right chest area of the user. The dispersal coverage areas will actuate as determined by the Control Centre.

Additionally, the connectivity of the Sensory Events to make a Sensory Event Array is adaptable through software. This allows developers the ability to virtually connect Sensory Events together to make a Sensory Event Array to easily implement the applications desired Sensory Signatures (or combination of Sensory Stimulations) and Sensory Outcomes. Furthermore, by making the connection virtual it allows the hardware integration to be determined by the hardware developers so it best suits the device the technology is integrated into. This ensures that the hardware integration and software integration of the Sensory Events does not limit the usefulness or determine the use of the technology.

Greater immersive Sensory Stimulation and virtual world awareness may be created through the perception of real-world sensations. The real-world environment provides a multitude of sensations depending on what an individual's senses receive. These triggered Sensory Stimulations allow an individual to effectively perceive our world and the things that are in it. Everything in the world has various forms of sensory feedback that they can provide an individual with which makes up their “Sensory Signature” which may be a particular combination of Sensory Stimulations. For example, the C-5 Galaxy (a military aircraft) has a particular Sensory Signature that would make it unmistakable. Even without being able to see the C-5 Galaxy, the sound, pitch, vibration and overall sensation that one feels when the plane is flying overhead would make it unmistakable and easily identifiable. Such real world signatures can be transferred to the gaming realm or other types of virtual reality. A gaming example; if an individual is playing a zombie survival game and hears particular noises of shuffling of feet, strange groans or just outright being attacked by zombies and feels something touch your back or grab you, the Sensory Signature that a zombie has would help identity whether particular sensations are from a zombie or an ally. This could provide enough sensory information to determine an effective plan of action. Or, the individual's avatar is moving backwards away from gun fire and is stopped because it backed up into something. The individual can instantly feel it on their back and make immediate adjustments. This in game decision making also translates into greater competence during game play as each more intuitive decision leads to greater “in game” success. This same methodology applies to movies as well as training simulations. In addition, sensation rehabilitation can be applied to traumatic accident, stroke or burn victims for example, whereby their nerves and brain can relearn through the Sensory Signature applications, especially though electrical stimulation. In addition, children who have no perception of various sensations due to their disabilities may learn through Sensory Signature applications.

The embodiments shown herein may allow for the reliability of Sensory Event and Sensory Signature outcomes. It creates this reliability in its consistent reproduction of outcomes as provided through the repetition of applications. Reliability refers to the consistency in the reproduction of the Sensory Event and the subsequent Sensory Signature for the user. For example, if a severe leg burn victim has limited feeling in their leg and physical/physiotherapy is required over a period of time, the sensual stimulation must be repeated and must be consistently applied through a repetitive process in order to produce reliable desired results.

In addition, embodiments have technological interoperability. Technological interoperability refers to the device's ability to operate between fields of use. This interoperability includes the ability of an embodiment to work in these other fields. For example, someone playing a video game can wear the device and receive stimulation as per the communications protocol set out in the Control Centre specifications. On the other hand, that same individual may come home from work and find they have a shoulder muscle that is tight and needs massaging and may use the device as per communications protocol initiating from computing device or may use the device as per the communications protocol initiating from computing device. The device he or she wears for video games thus can also be worn for this purpose as well, physiotherapy. Alternatively this person may want to go to the movies or partake in a training simulation where the same device is also worn. As mentioned previously, only the Decoder potentially needs to be altered when changing platforms. One could do this via physically changing the Decoder 56, or alternatively by changing the software of the Decoder 56.

The combination of the components detailed herein results in various embodiments with various unique and innovative features. It allows for a complete and more holistic experience that encompasses more of one's senses than just video and stereo-audio. Examples include: a player is provided surround sound and hears (as well as sees) bugs crawling on their character and receiving through Sensory Manipulation the Sensory Signature of something crawling on their stomach through a Sensory Event Array as provided by vision, EMS, vibration and sound. Another example would be military training whereby surround sound gives directional feedback from an explosion and the Sensory Signature is created by vision, sound, muscle stimulation, vibration, force/physics, air blast and constriction/compression simulating shrapnel entering the soldier's body and the concussive force of the explosion. Additionally, a blind person walking in a city is given directional cues either audibly or physiologically with the other used as a proximity warning for close or closing obstacles or dangers. A theatre goer is sitting in a movie theater wearing the device where the surround sound provides directional sound with the sensory stimulation components providing stimulation to the viewer's body as the main character of the film is experiencing it in the movie.

FIG. 14 depicts a potential pathway from the initiating device 54 that the signal takes to get to an actuator. The initiating device 54 may provide an input module. The initiating device 54 is a computing device that uses software to collect sensory related data and create control data that will determine what physiological stimulation each pair of electrodes 10 or other actuator will create at any given point in time. A computing device that allows for the inclusion of software and the output of data that the software has generated can be used as an initiating device 54 for the system. The Control Centre 16 is the component of the system which controls the signal, duration, strength, and/or pattern of the electrical stimulus generated causing or activating a Sensory Event, whether singularly, in a Sensory Event Array, random or other formation. The Control Centre 16 may provide an input module for collecting sensory related data. A Decoder 56 can potentially be operably connected between the initiating device 54 and the Control Centre 16. The Decoder 56 is used to decipher or transform the data being sent from the initiating device 54 into a format compatible with the Control Centre 16. This deciphered data is then sent from the Decoder via a communications protocol to the Control Centre 16. Input data to trigger different Sensory Events may be collected through an input module coupled to Decoder, initiating device 54, or Control Centre 16. Actuators are shown such as vibration 48, constriction/compression 50, air flow 51, temperature 53 and force/physics 55.

There may optionally be an additional computing device between the actuator and the Control Centre 16 that is actuator specific 12a such as an EDA, but may also include physiological data acquisition, EEG, ECG, respirations, pulse, blood pressure and temperature. Another example is the MCEIATR 12 which sends the electrical impulse to the electrodes 10 and subsequently the Sensory Manipulation or a Sensory Event, whether singularly, in array, random, and so on. The determination on which pairs of electrodes 10 are activated and the level, duration, strength and or pattern that each electrode pair 10 will produce is based on the sensory related data received from a Decoder 56.

In addition, the data can be sent from a MCEIATR 12 alone, or as described before an initiating device 54 can initiate the process through a virtual medium or device through the Decoder 56 to the Control Center 16 which is operably connected to the MCEIATR 12 which sends electrical impulses to the electrodes 10. The MCEIATR 12 may optionally also be defined in the garment 14.

In the one embodiment, parts of the garment 14 are electrode conductive as to give an effective, wireless electrical pathway between a MCEIATR 12 and an electrode 10, while other parts are not conductive as to inhibit certain circuits and to control the areas that are being stimulated. Therefore, in this embodiment it may also be advantageous to include wired electrical pathways 18 between some MCEIATRs 12 and some electrodes 10. In both these connections, the MCEIATR 12 is operably connected to at least one pair of electrodes 10.

In one embodiment, some or all of the components including; the electrodes 10, computing device 54, MCEIATR 12, control center 16, initiator 54 are removable from the garment 14 as to allow for repair, instrumentation calibration, replacement, battery replacement, cleaning or other general maintenance (henceforth referred to as maintenance). They may be attached or fastened to the garment 14 via using an adhesive technology. Adhesive technology consists of any technology that allows for the removal of electrodes 10, other actuators or other components for maintenance. It may be one or more of; VELCRO®, hook and loops or clasps or pouches. However, the list is just exemplary and should in no way be interpreted as limiting.

For various embodiments, the Decoder 56 may need modification depending on the initiating device 54 being used. In one embodiment, one can buy a new Decoder 56 for every initiating device 54 the person wishes to connect to the garment 14. Alternatively, one could alter the programming of the Decoder 56 meaning an individual may only need to install software or a patch to move to a different initiating device 54. However, if a platform were designed to output data consistent with what is read by the Control Centre 16, the use of a Decoder 56 would not be necessary and no changes to the hardware or software would be necessary with switching to the designed initiating device 54.

The power source (Power Regulator) 46 for the device may be any source that effectively allows the function of the device. This may include, but is no way limited to; rechargeable batteries, replaceable batteries or directly wired into a power source such as an outlet, or a combination thereof.

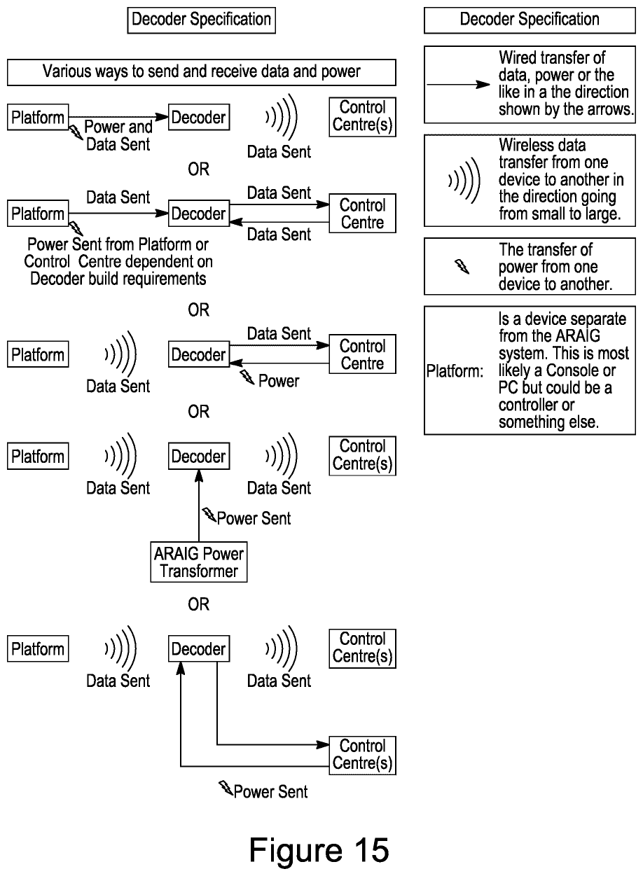

Referring now to FIG. 15 there is shown an example of various Decoder specifications, along with a legend depicting various elements of this and other figures and various ways to send and receive data and power.

A Decoder 15-1 may be capable of receiving the needed sensory related data wired or wirelessly from the initiating device, or other computing device 15-2. A Decoder 15-1 may be capable of altering or transforming the data sent from the initiating device 15-2 into data that is then sent wired or wirelessly to the Control Centre 15-3 to activate the Exoskeleton's Nervous System appropriately.

A Decoder 15-1 may be capable of receiving software updates via a platform computing device 15-2 and from the Control Centre 15-3. A Decoder 15-1 may be capable of updating the software of the Control Centre 15-3 if its software is outdated. A Decoder 15-1 may be capable of receiving its power from a Control Centre 15-3 when attached to a Control Centre 15-3 or computing device 15-2 when attached to a computing device 15-2 but when completely wireless for both sending and receiving of data it needs to be attached to the Power Transformer for power. There may be a different Decoder 15-1 for different computing device platforms (e.g. PS4, PC, Xbox360, Xbox One) and more as they continue to come to market or as we enter different markets.

A Decoder may be designed specifically for one or more forms of transmission to work with a particular platform (wired and Bluetooth™, etc.). If multiple devices use the same data transfer protocols it is possible for some Decoders to work for several platforms.

Decoder 15-1 is designed to receive data from a platform 15-2 either wired, wirelessly or both. Each Decoder may be able to physically connect to a Control Centre 15-3 to send the Data to that Control Centre and may be able to send the data wirelessly to one or more Control Centres simultaneously; the latter may not require physical connection to a Control Centre. The wired and wireless transmission of data to one or more Control Centres may be the same for each Decoder while the wireless and wired transmission from a platform may be specific to each platform, although all Decoders will be able to connect to a PC. Thus, there will be a variety of Decoders designed to receive Data from various platforms.

For a Control Centre to receive data from a Decoder to Decoder may first be synced with the Control Centre. Once synced a Decoder can then be used wired or wirelessly for that particular Control Centre. Multiple Control Centres can be synced to the same Decoder to receive the same information wirelessly. Each Decoder may receive power to turn it on externally, either through the Platform, a Control Centre or ARAIG's Power Transformer 15-4. When wired it is connected to the device physically. This physical connection will most likely be via a USB 15-5 for ease of use. When wireless the Decoder is either sending and/or receiving via one or more wireless protocols. A Decoder also can download software updates and patches via various Platforms (at least via a PC) when wired to that Platform. Also, updates and patches can be sent or received from a Control Centre.

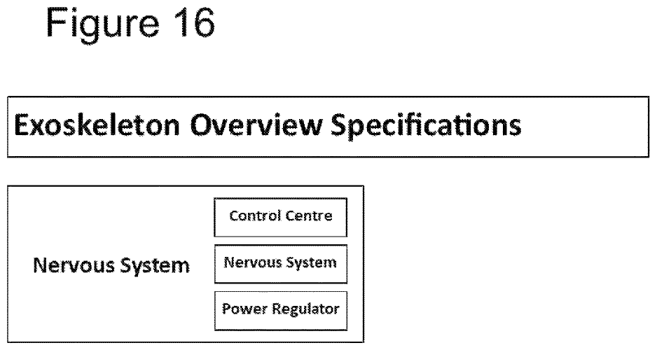

FIG. 16 illustrates exoskeleton specification according to some embodiments. The example exoskeleton may include an upper body suit that covers the torso, shoulders and upper arms that has all of the core functionality working and integrated where required. The exoskeleton may include a Control Centre (e.g. Power Button (Integrated), Mem Chip (Detachable), Profile Selector (Integrated), Receiver (Integrated)), a Nervous System (e.g. Vibration Components (Integrated), STIMS (e.g. Medically Compliant Electrical Impulse Amplifier Transmitter Receiver(s) (MCEIATRs) (Integrated), Paired Electrodes (Integrated), Electrode Pads (Detachable)), Surround Sound (e.g. External Emitter/Receiver (Detachable), Receiver (Integrated), Amplifier (Integrated), Speakers (Integrated), Microphone Jack (Integrated), Microphone (Detachable), Transmitter/Audio Out (Detachable))), and a Power Regulator (e.g. Power Plug (Detachable), Power Transformer (Detachable), Charger/Power Receiver (Integrated), Wiring to all of the necessary components to power all the components of the Exoskeleton via the activation of the Receiver(s) (Integrated), Power Cell (Detachable)).

For an Exoskeleton to work with any other platform the development of a new Decoder. Surround Sound External Emitter/Receiver and Surround Sound Transmitter/Audio Out may require some alterations. All of which are detachable components to allow the Exoskeleton to remain universal.

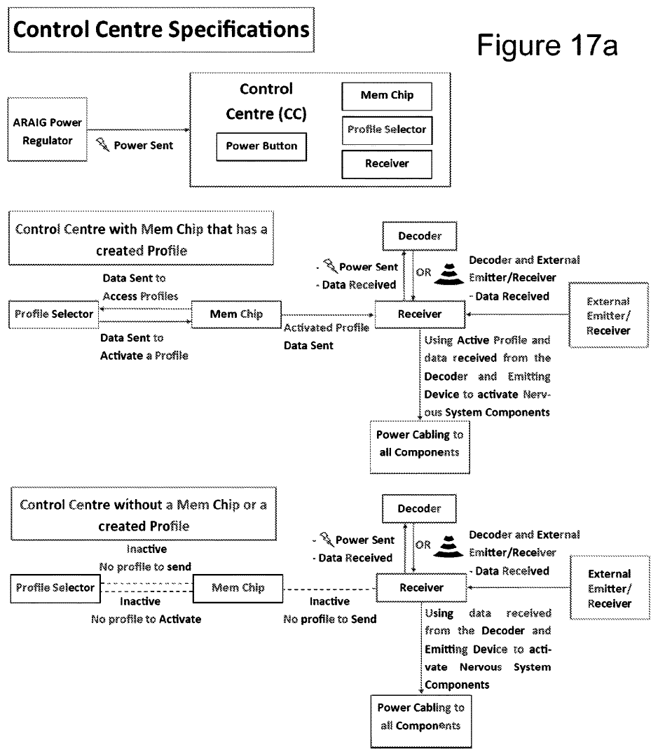

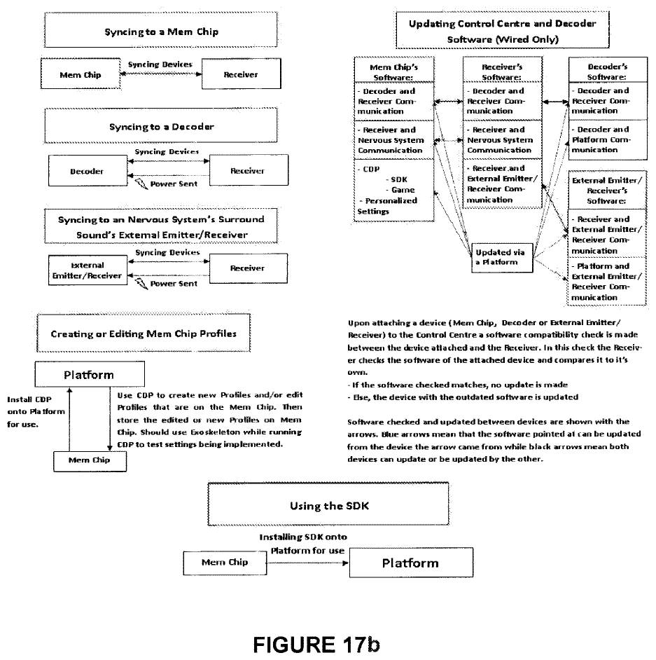

The Control Centre may be operable for updates or alterations in parallel with other component updates, software updates and patches, and hardware system changes. The Nervous System may be operable for alteration and advances of current components, creation of new components i.e. constriction/compression, force/physics, air. The Power Regulator may be operable for consumption efficiency, power reduction, power weight, power Placement. The exoskeleton may include a variety of design modifications including placement and specifications of its components, creating an Exoskeleton for particular niche markets, modular design for the Exoskeletons, variants in different sizes and for different sexes, and so on. FIGS. 17a and 17b illustrate Control Centre specifications according to some embodiments.

A Control Centre may have a Power Button 17-1 which is capable of turning the Exoskeleton on and off to receive power from a Power Regulator 17-2. A Control Centre may have a Mem Chip 17-3 which has the CDP (Calibration Diagnostic Protocol), Personalized Settings, Decoder and Receiver Communications, and Receiver and Nervous System Communication Software. The CDP is able to install/download the SDK onto the computing device for use and the game or other software onto various platforms. The SDK allows developers to easily program, test and integrate the system into their software. The Game or other software may allow a wearer of an Exoskeleton to properly adjust the profile settings and create multiple profiles to have different types of immersive experiences. The personalized setting can be adjusted on computing device through the Game. A Mem Chip may be able to receive software updates via a platform (e.g. computing device) and from the Control Centre. A Mem Chip may be able to update the Control Centre software if outdated. Mem Chip may attach, sync and communicate with devices via USB.

A Control Centre with a Mem Chip that has a created Profile 17-001 may have a Profile Selector 17-4 which is able to go through the various saved profiles on an attached Mem Chip 17-3. Upon selecting a profile, a Profile Selector 17-4 may have sensory feedback that specifies the one selected. A Profile Selector 17-4 may have the Mem Chip 17-3 send the selected profile to the Receiver.

A Control Centre without a Mem Chip or a created Profile 17-002 may have a Receiver 17-5 which has the CDP, Personalized Settings, Decoder and Receiver Communications, and Receiver and Nervous System Communication Software. Using the Decoder 17-6 and Receiver Communication software, the Control Centre may be capable of receiving the raw non-audio sensory activation data from the Decoder. Using the Receiver 17-5 and External Emitter/Receiver Communication software, the Control Centre may be capable of receiving the raw audio activation data from the External Emitter/Receiver 17-7. Using the Receiver and Nervous System Communication software, the Control Centre may be capable of taking the received data from the Decoder, External Emitter/Receiver and Mem Chip active Profile to activate the appropriate Nervous System components at the proper intensities and locations. The Control Centre may be able to receive software updates via a Mem Chip, Decoder and External Emitter/Receiver. Also able to update the software of Mem Chip, Decoder and External Emitter/Receiver.

In example embodiments, the Control Centre may have three USB ports to attach a Mem Chip, Decoder and Surround Sound External Emitter/Receiver for syncing and wired data transfer. Which USB port is used by each detachable device may not matter.

In example embodiments, the Control Centre may have a wireless receiver to be able to receive data wirelessly from a Decoder.

In example embodiments, the Control Centre may have a wireless receiver to be able to receive data wirelessly from Surround Sound External Emitter/Receiver.

In example embodiments, the Control Centre may have Activate all the needed nervous system components based on the data it receives from the Decoder and Surround Sound.

In example embodiments, the Control Centre may need to be integrated into the exoskeleton in such a way that it does not restrict movement and that it is easily accessible to add or remove any of its detachable components (Mem Chip) or components that can be attached to it (Decoder and Surround Sound External Emitter/Receiver) without the wearer having to take off the Exoskeleton.

In example embodiments, the Control Centre may be removable and replaceable for defect or upgrade or fixing, and so on.

Transmitter/Audio Out component currently may be part of the Nervous System Surround Sound or built into the Control Centre. It may be a detachable component of the Control Centre.

In example embodiments, the Control Centre may have wireless transmission protection from Decoder to Receiver and wireless transmission interference reduction.

The Power Button is the component to turn on the ARAIG Exoskeleton. Once on, the Control Centre will be able to function as described in each of its components and power will be able to flow throughout the Exoskeleton as required.

Mem Chip may be a detachable component of the Control Centre and may be a USB design for each of use in data storage and transfer. Mem Chip may contain the needed Calibration and Diagnostics Protocol (CDP) for creating profiles on various platforms. While creating a Profile on a given Platform 17-8 the Mem Chip 17-3 may be physically attached to the platform to receive the edited or new profile and not the Control Centre. To use the profiles stored on the Mem Chip the Mem Chip may be attached to the Control Centre Receiver and the profile must be selected by the Profile Selector and the CDP is installed onto the Platform 17-8 for use. The CDP is used 17-006 to create new Profiles and/or edit Profiles that are on the Mem Chip as well as store the edited or new Profiles to the Mem Chip. The exoskeleton may be used while running CDP to test settings being implemented and or the CDP may provide a graphic or visual display of actuator settings allowing for a visual testing of settings being implemented.

In one embodiment, the SDK software 17-007 on the Mem Chip 17-3 can be installed/downloaded wired or wirelessly onto various platforms 17-8 for use by developers to properly integrate ARAIG into the own software.

Profile Selector may be a component of the Control Center that allows an individual to cycle through their created profiles via physical inputs. If they do not have a Mem Chip attached or they have no profiles it is inactive. If there are any profiles then by default it activates the newest profile when the Control Centre is powered on. Afterwards a user can cycle through the profiles and select a different profile to activate. Each profile may be saved with sensory feedback so the user can easily differentiate between the profiles while searching. When updating the Control Centre and Decoder Software (wired only) 17-008, the Mem Chip, Receiver and Decoder carry out the process as shown in the figure. Upon attaching a device (Mem Chip, Decoder or External Emitter/Receiver) to the Control Centre a software compatibility check is made between the device attached and the Receiver. In this check the Receiver checks the software of the attached device and compares it to it's own. If the software checked matches, no update is made, else, the device with the outdated software is updated.

Receiver is a component that has a receiver to pick up wireless transmissions from any Decoder or Nervous System Surround Sound External Emitter/Receiver. Receiver is the component that is directly connected to by a wired Mem Chip 17-003, Decoder 17-004 and/or a Nervous System's Surround Sound's External Emitter/Receiver 17-005 through ports (e.g. 3, USB) and has software to use the synced Mem Chip profiles, Decoder date and External Emitter/Receiver data to activate the necessary Nervous System components.

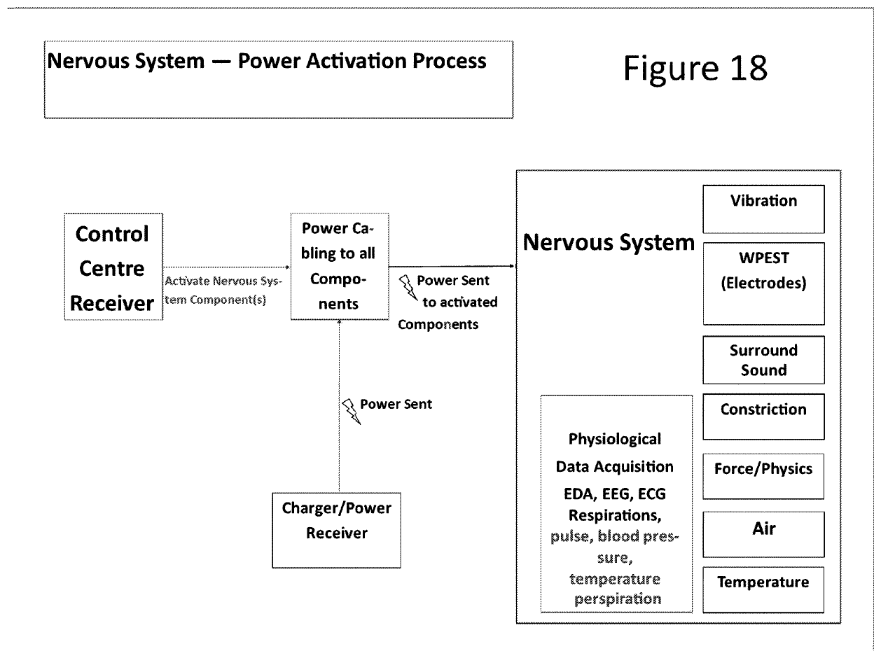

FIG. 18 illustrates nervous system specifications for power activation according to some embodiments. This illustrative example may help visualize the flow of power to the Nervous system as a whole.

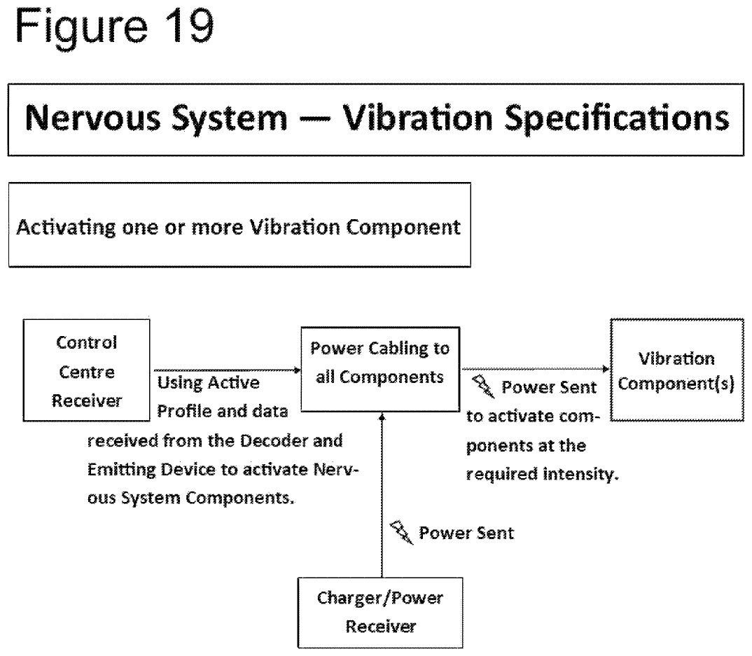

FIG. 19 illustrates nervous system specifications for vibration according to some embodiments when activating one or more vibration components.

In example embodiments, the Nervous System vibration device may include enough vibratory stimuli to have a coverage area of the torso front and back, shoulders and upper arms. The amount of vibratory stimuli required to do this may be determined depending on application and field of use. In some examples there may be a minimum of 16 front, 16 back, 8 left should/upper arm and 8 right shoulder/upper arm; total of 48 points; although most important factor is coverage over amount of stimuli. This is a non-limiting example.

Each vibratory stimulus may be able to create different ranges of intensity from a small vibration to an intense shaking sensation in their own location.

Each vibratory stimulus may be able to activate individually, sequentially of other vibratory stimuli or sensory feedback devices, or simultaneously of vibratory stimuli or sensory feedback devices; all of which can also be for different durations and different coverage areas.

The nervous system may be programmed with algorithms created to give sensations such as a single location, multiple locations, a region, expansion or contraction of impact in an area, vibration in a line all at once or in sequence and a wave sensation; all at varying intensity and duration.

The activation of a vibratory stimulus will not cause interference with the activation of other stimuli; such as other vibratory stimuli, STIMS or Surround Sound.

Different embodiments may have variation of placement, intensity, duration and type of vibratory stimuli. Different embodiments may consider user's ability to localize such sensations and what the sensations feel like to them. Different embodiments may have different updates to algorithms to create a variety of sensations. Different embodiments may use a device that is compliant with all standards but specific to a field of use; may create several variations depending on market niche.

Vibration Components are the multitude of vibratory stimuli devices that are integrated throughout the Exoskeleton. Each vibration device is capable of working at various intensities to create different vibratory sensations.

A coverage area that an individual should feel the vibratory stimuli may be the torso front and back and the upper arms and shoulder areas of the Exoskeleton. The amount of vibratory stimuli devices to cover these areas may allow for an individual to feel both localized sensation and moving sensations from one vibratory stimuli device to one or more other vibratory stimuli devices.

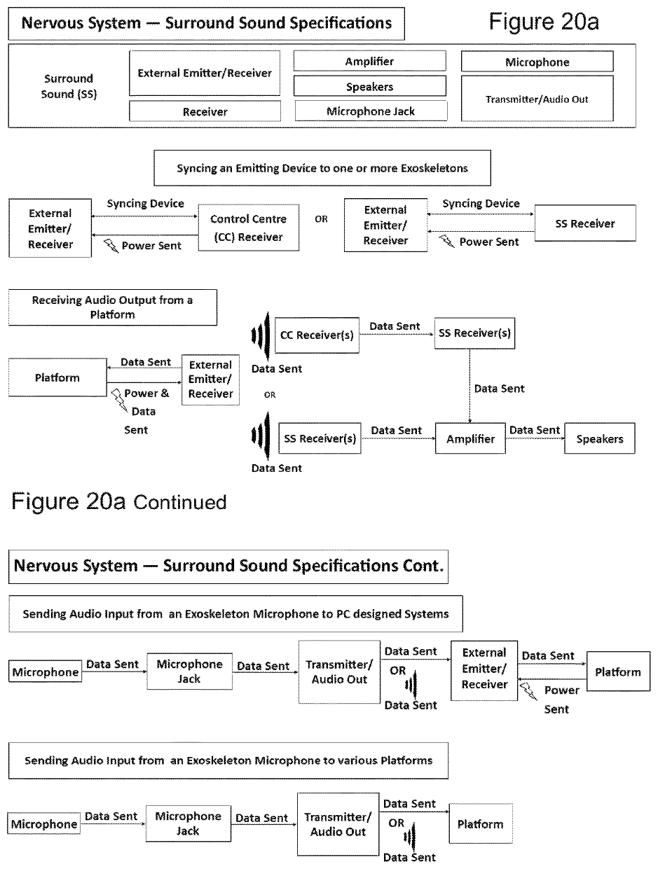

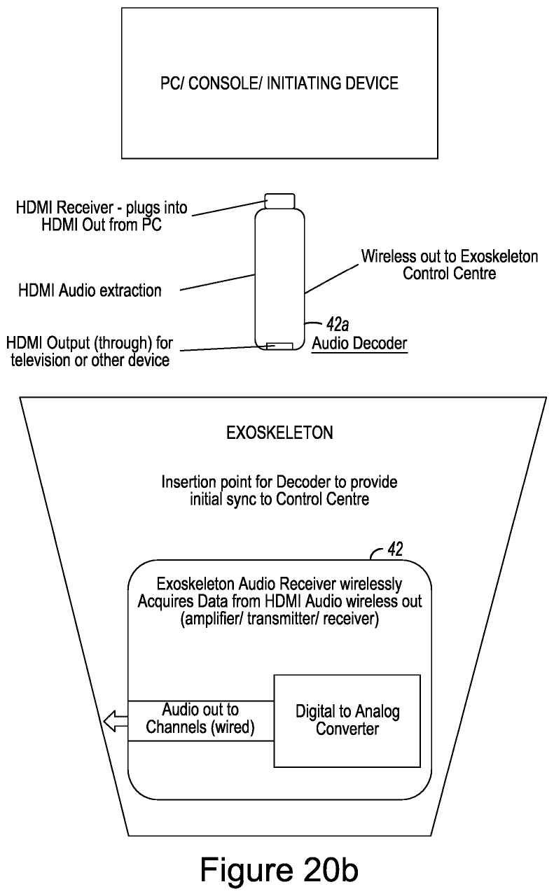

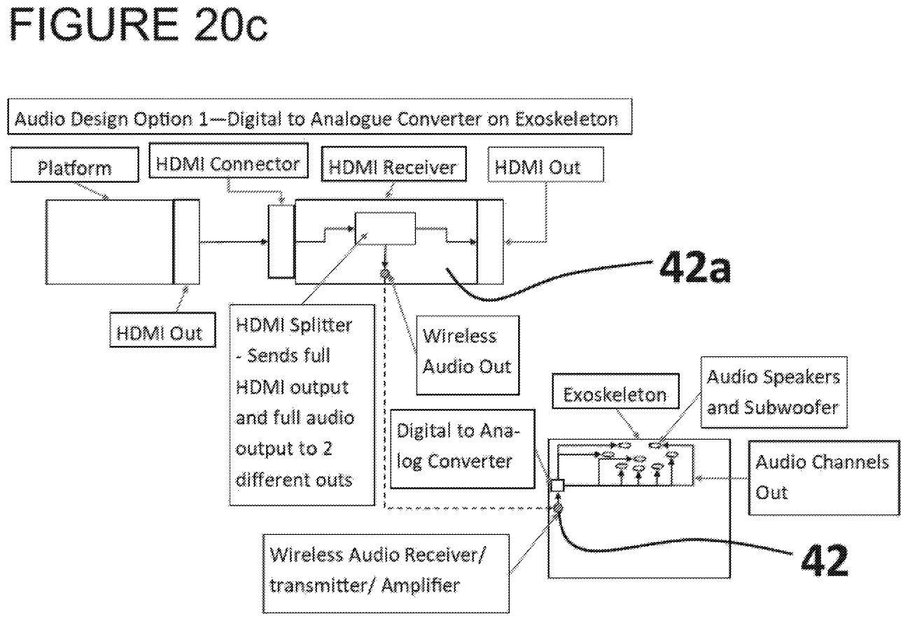

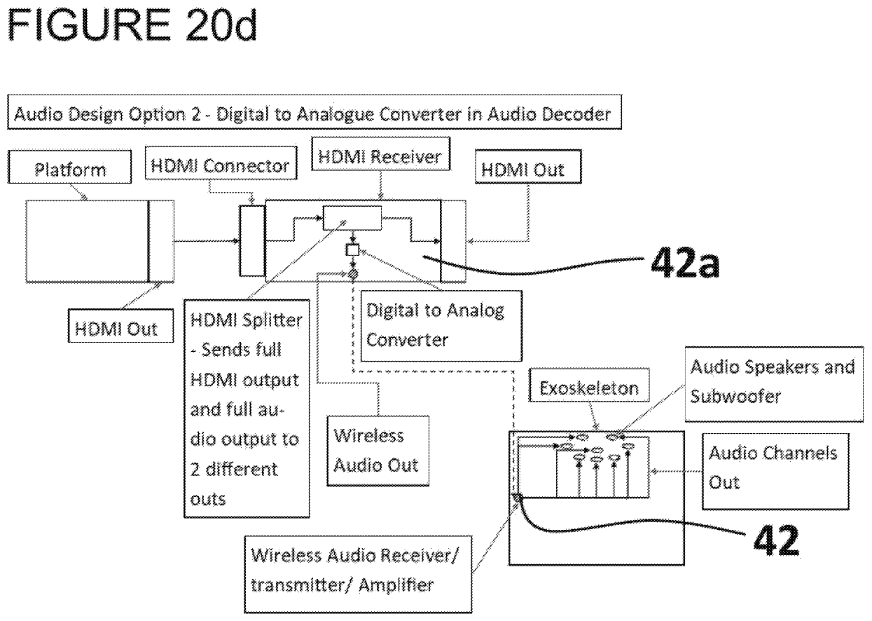

FIGS. 20a to 20d illustrate nervous system specifications 20-001 for surround sound according to some embodiments. In one embodiment, there is shown syncing an Emitting Device to one or more Exoskeletons 20-002. In another embodiment, there is shown receiving audio output from a platform 20-003. In another embodiment, there is shown sending audio input from an exoskeleton microphone to PC designed systems 20-004, in another embodiment, there is shown sending audio input from an exoskeleton microphone to various platforms 20-005.

In example embodiments 20-006, Nervous System surround sound sensory devices may include an External Emitter/Receiver Component or Audio Decoder 42a capable of receiving Audio Output from the computing device and send it to one or more Exoskeleton's that are synced to receive the data from this External Emitter/Receiver. The Emitter/Receiver Component may be synced with Surround Sound Receiver(s) or Control Centre Receiver(s) for one or more Exoskeletons to receive the Audio data. Emitter/Receiver Component may receive the data directly from the External Emitter/Receiver. Emitter/Receiver Component may be able to receive software updates via a platform computing device and from Control Centre Receivers; also enabling the update of the software of the Control Centre Receiver if its software is outdated. Emitter/Receiver Component may update and be updated by a Control Centre Receiver if the Control Centre Receiver is the device that has been determined during development to sync with the External Emitter/Receiver. Emitter/Receiver Component may be wired to directly to receive Microphone Audio from the Exoskeleton via the Transmitter/Audio Out and thus would be wired to a platform to send the Microphone Audio to it. Emitter/Receiver Component may receive its power from the platform or Exoskeleton when syncing.

In example embodiments as per FIG. 20d, Nervous System surround sound sensory devices may include a Receiver. It takes the Audio Output of an initiating device wirelessly from Emitter/Receiver or Audio Decoder via Wi-Fi, Bluetooth™, radio, et cetera, The receiver may translate the digital data to analogue 20-007 and send it to the amplifier.

In example embodiments, Nervous System surround sound sensory devices may include an Amplifier that takes audio data from the Receiver and distributes it appropriately to the various speakers located on the Exoskeleton.

In example embodiments, Nervous System surround sound sensory devices may include Speakers. The exact angle and positioning of the speakers may be dependent on the field of use and application.

In example embodiments, Nervous System surround sound sensory devices may include a Microphone as the Audio Input device for the wearer of the Exoskeleton and a Microphone Jack that can be used to attach a microphone and sends Microphone Audio to the Transmitter/Audio Out component.

In example embodiments, Nervous System surround sound sensory devices may include a Transmitter/Audio Out that receives the Microphone Input and sends it either wirelessly to a platform or wired or wirelessly to the External Emitter/Receiver. This piece may be built into the Control Centre instead should that be decided during development.

In example embodiments, Nervous System surround sound sensory devices may include variations on placement, volume and speaker quality based on a user's ability to localize sound, for example. In example embodiments, Nervous System-Surround Sound may include updates to algorithms to transfer sound effectively between speakers.

External Emitter/Receiver is a device that may not be integrated into the Exoskeleton. To use, it may be wired to a particular platform to receive the audio output from the connected platform and in specific circumstances receive microphone audio input from a Transmitter/Audio Out. When the audio output is received from a platform it is sent out to one or more Exoskeletons' Receiver that has been synced with the External Emitter/Receiver. To sync a Receiver to an External Emitter/Receiver the Emitting Device needs to be wired to the Receiver. Once synced the Receiver will be able to receive the data output from the Emitting Device. The Emitting Device receives its power from the platform or Receiver that it is attached to.

The Emitting Device may Sync with the Control Centre's Receiver rather than the Nervous System's Surround Sound's Receiver and the Control Centre would then send the data to the Nervous System's Surround Sound Receiver. This may be implemented during the development process. With that stated it would also allow the Mem Chip to set the Surround Sound settings which in turn would maintain a consistent flow of external data to Control Centre to Nervous System Activation.

Receiver is the component that receives the data from a synced Emitting Device and sends the data to the Amplifier(s).

Amplifier is the component that receives data from the Receiver to activate the appropriate speaker(s) to play the proper localized sound for the wearer.

Speakers are the multitude of sound components that create the localized audio for the user. The placement of the speakers creates the surround sound effect. The speakers that are activated and the sound that is created from each speaker is de pendent on the data that is received from the Amplifier(s).

Microphone is a detachable component that the user will use to input audio into an Exoskeleton's Nervous System via a Microphone Jack to be used by other systems or Exoskeleton's as audio output.

Microphone Jack is the component that allows a user to connect any microphone they would like to use for audio input. Upon receiving audio input from a Microphone the data is sent to the Transmitter/Audio Out to be sent out for use by other systems (Platforms and External Emitter/Receivers),

Transmitter/Audio Out is a detachable component that will receive audio input from the Microphone Jack and send the audio input to a platform or device that has been synced wired or wirelessly to be used as audio output. As the wireless and wired transmission to each platform could differ there will be a variety of Transmitter/Audio Outs for the various platforms.

The Control Centre may have the Transmitter/Audio Out component attachable to it. Thus, the Microphone Jack may send the Audio input from the Microphone to the Control Centre's Transmitter/Audio Out device to send out to the particular systems (Platforms and External Emitter/Receivers),

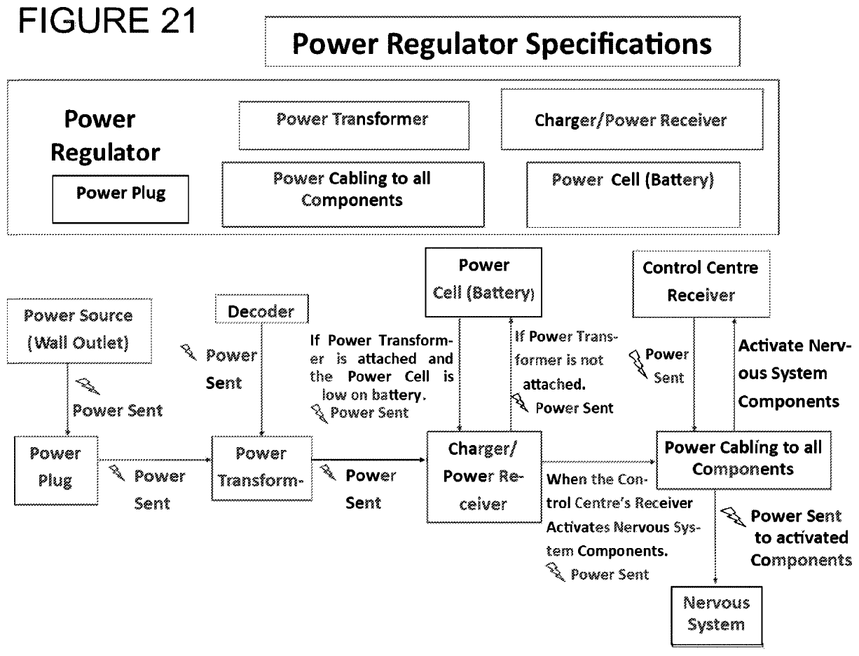

FIG. 21 illustrates power regulation specifications according to some embodiments.

In example embodiments, Power Regulator 21-001 may include a Power Plug 21-002 that can be used with wall outlets and capable of plugging into a Power Transformer 21-003 to send power to various components.

In example embodiments, Power Regulator may include a Power Transformer as a universal power receiving device to convert the power to the appropriate amount to power an Exoskeleton, Power Cell 21-004 and Decoder 21-005 individually, several, or all need simultaneously without affecting the use of the other devices. This may be plugged into by any Power Plug designed for the system no matter the Power Plugs specifications to a particular countries wall outlet power output, for example.

In example embodiments, Power Regulator may include a Power Cabling to All Components 21-006 via integrated wiring connecting all components to power entire Exoskeleton system. There may be power through cabling is controlled by the Control Centre Receiver.

In example embodiments, Power Regulator may include a Charger/Power Receiver 21-007 that is able to distribute the needed power to all components of an Exoskeleton and a Power Cell Simultaneously. This may be able to use an attached Power Cell to distribute the needed power to all components of an Exoskeleton Simultaneously, and be able to receive power from a Power Transformer for wired use. This may be built into the exoskeleton in such a way that it does not restrict movement and that it is easily accessible to plug in the Power Cord and remove/replace/attach a Power Cell by the wearer without having to take off the Exoskeleton.

In example embodiments, Power Regulator may include a Power Cell developed or acquired the needed battery to provide an Exoskeleton with a reasonable battery life. When attached to the Exoskeleton's Charger/Power Receiver the Power Cell may be able to provide power to the Exoskeleton or be charged by a power Transformer attached to the Charger/Power Receiver.

In example embodiments, Power Regulator may include a standalone multi battery charger developed or the specifications determined for manufacture of such a device.

In example embodiments, Power Regulator may include power reduction and power efficiency mechanisms.

Power Plug is a component to supply the Power Transformer with power through a wired connection. There may be several Power Plug variants to deal with the different electrical power systems and their outputs as they vary from country to country.

Power Transformer is a component that takes the power it receives from the Power Plug and ensures it meets the needed power requirements to charge and/or power the ARAIG Suit and its components. It also can be directly plugged into by the Decoder to be used as the Decoder's external power source.

Charger/Power Receiver is a component that powers the ARAIG suit and its components. It receives its power from the Power Transformer or a Power Cell (Battery). If there is no attached Power Cell it receives its power from the Power Transformer. If there is a Power Cell attached and no Power Transformer attached it receives its power from the Power Cell. If a Power Cell and Power Transformer are attached it receives its Power from the Power Transformer and diverts energy to charge the Power Cell until it is fully charged by the Power Transformer. Which nervous system components are activated and at what intensity they are activated are dependent on what the Control Centre Receiver allows to be activated; the Charger/Power Receiver supplies the power for the specifics to occur.

The Charger/Power Receiver can recharge a Power Cell without the Exoskeleton being turned on.

Power Cabling to all Components provides the wiring to give all the components of the exoskeleton power and indirectly a wired Decoder power through the Control Centre. When the Exoskeleton is powered on, the Control Centre is the only component that the Power Cabling to all Components that is always powered on.

Power Cell (Battery) is a detachable component that allows the Exoskeleton to be wireless. When attached to the Charger/Power Receiver it can give the Exoskeleton the needed power to operate. It can also be recharged directly through the Charger/Power Receiver if the Charger/Power Receiver is receiving power from the Power Transformer instead.

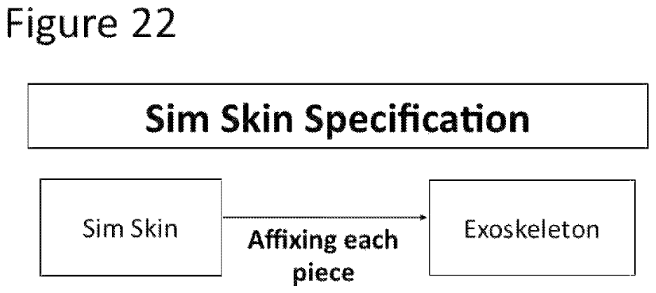

FIG. 22 illustrates wearable material specifications according to some embodiments.

The wearable material may be referred to as “sim skin”, for example.

The Sim Skin may cover the majority of an Exoskeleton's Torso front and back, shoulders and upper arms (e.g. 4 to 6 separate pieces). Sim Skin components may be designed specifically for males or females, and some components may work for both males and females (e.g. varying sizes, while some will be used only by a particular gender). The Sim Skin may be affixed to an Exoskeleton. The components to affix the Sim Skin components to the Exoskeleton may blend with the aesthetic look and design or may be able to be easily hidden while the Exoskeleton is worn. The Sim Skin may not hinder or negatively affect the wearer's mobility, comfort, ergonomics or functionality of the suit. There may be different sizes or a one size fits all.

Sim Skin design may allow the Sim Skin Torso component(s) to be affixed or removed without the wearer having to take off the Exoskeleton. Furthermore, if possible the Sim Skin components may allow easy access to the Exoskeleton detachable and interactive components without removal of the Sim Skin components or with only partial removal of one or more of the Sim Skin components so that the wearer does not have to take off the Exoskeleton. There may be alternative colours, designs, materials, components, accessories/attachments. There may be increased modular design for the Sim Skins, such as possible Female, Male and Unisex Sim Skin components and sizes.

Each Sim Skin may have several aesthetic components. These aesthetic components are affixed on top of an Exoskeleton to create a particular look. Components from several Sim Skin's can be affixed to an Exoskeleton to give users an even more unique look. Each of the pieces cover a different portion of the Exoskeleton; such as the front and back of the torso and each shoulder/upper arms. Although, the exact coverage, placement and number of components will be dependent on the most effective design to do so.

The number of components may be determined through the development of the Sim Skins but there needs to be enough components to cover the majority of the front and back of the torso, and each shoulder/upper arm without hindering or negatively affecting the wearer's mobility, comfort, ergonomics or functionality of the other ARAIG components, especially the Exoskeleton.

The components of the Sim Skin that affix each piece to the Exoskeleton if visible need to match the aesthetics of the Sim Skin and/or that of the Exoskeleton. Otherwise, if the components of the Sim Skin that affix to the Exoskeleton are able to be hidden easily it does not matter. A Sim Skin can be easily attached to or taken off by easy-to-use components for affixing the Sim Skin to the Exoskeleton.

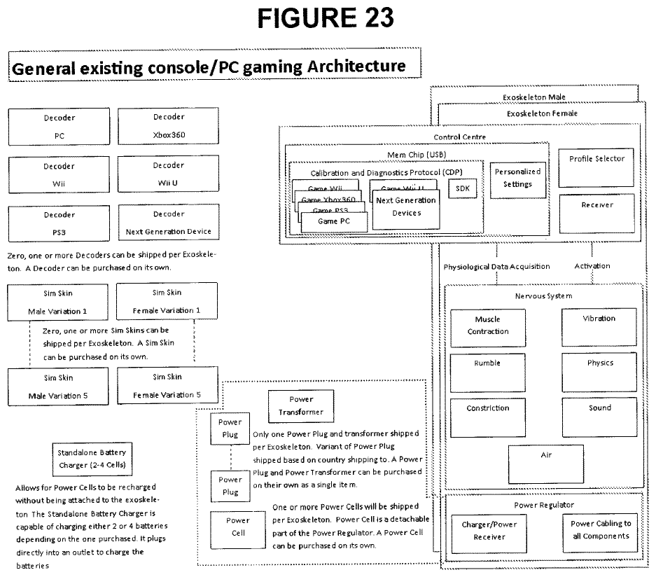

FIG. 23 illustrates an example gaming existing console/PC gaming architecture according to some embodiments. As shown in the Figure, zero, one or more decoders can be shipped per exoskeleton. A decoder can be purchased on its own. Several consoles each with a decoder are shown including a PC, Wii, PS3, Xbox360, Wii U and a next generation device. Also depicted is a Sim Skin male variation 1 through 5 and a Sim Skin female variation 1 through 5. Zero, one or more Sim Skins can be shipped per exoskeleton. A sim skin can be purchased on its own. Also depicted is a standalone battery charger (2-4 cells) which allows for power cells to be recharged without being attached to the exoskeleton. The standalone battery charger is capable of charging either 2 or 4 batteries depending on the one purchased. It plugs directly into an outlet to charge the batteries. The control centre is depicted in both the male and female exoskeleton versions. Also depicted is the power transformer with power plug and power cell as well as the power regulator. In this embodiment, only one power plug and transformer shipper per exoskeleton. Variant of power plug shipped based on country shipping destination. A power plug and power transformer cane be purchased on their own as a single item. One or more power cells will be shipped per exoskeleton. Power cell is a detachable part of the power regulator. A power cell can be purchased on its own. As described herein, the control center is in communication with the nervous system actuators acquiring physiological data or activating the actuators.

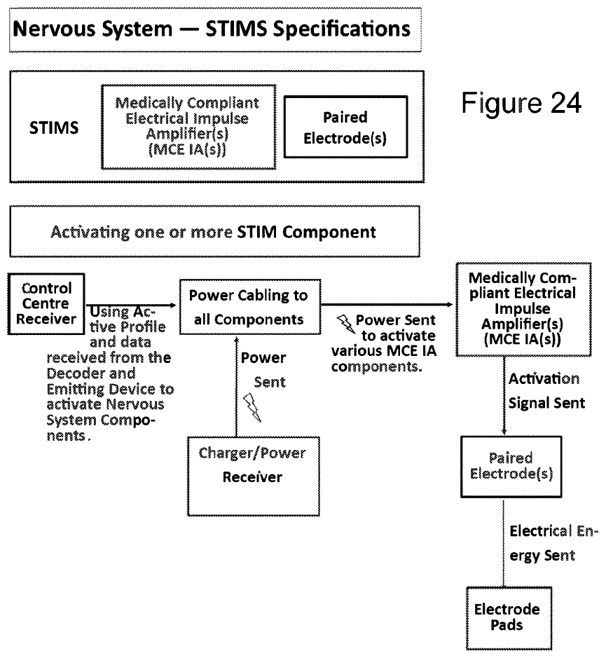

FIG. 24 illustrates example nervous system STIMS specifications. The STIMS includes MCEIAs 24-1 and paired electrodes 24-2.

Medically Compliant Electrical Impulse Amplifier(s) (MCEIA(s)) are the components that provide stimulation to a user's tissue, nerve and or muscle through electrical energy. They are medically compliant in their activation protocols and limitations and adhere to US FDA, Canadian, and European standards for such devices. The MCEIAs receive the necessary power from the Exoskeleton to send the needed signal to one or more Paired electrodes to stimulate the user's physiology.

The amount of MCEIA devices required in the Exoskeleton may be dependent on the amount of locations that one MCEIA can effectively provide stimulation simultaneously without compromising the effects that one location can receive and still being able to adhere to the activation protocols, limitations and standards across different nations.

Each Paired Electrode is integrated throughout the Exoskeleton. When activating one or more STIM component, each Paired Electrode 24-2 receives the necessary power to send and receive through the attached Electrode Pads 24-3.

There may be four Paired Electrodes of which two pairs may be used to cover the abdomen area while another two may be placed to cover the shoulder to chest area. The addition, removal or altering of the placements is possible.

Each Electrode Pad is attached to an Electrode. For every pair of electrodes the user places the Electrode pads onto a single muscle. When the Electrode Pads receive power the muscle they are attached receives a particular electrical stimulation.

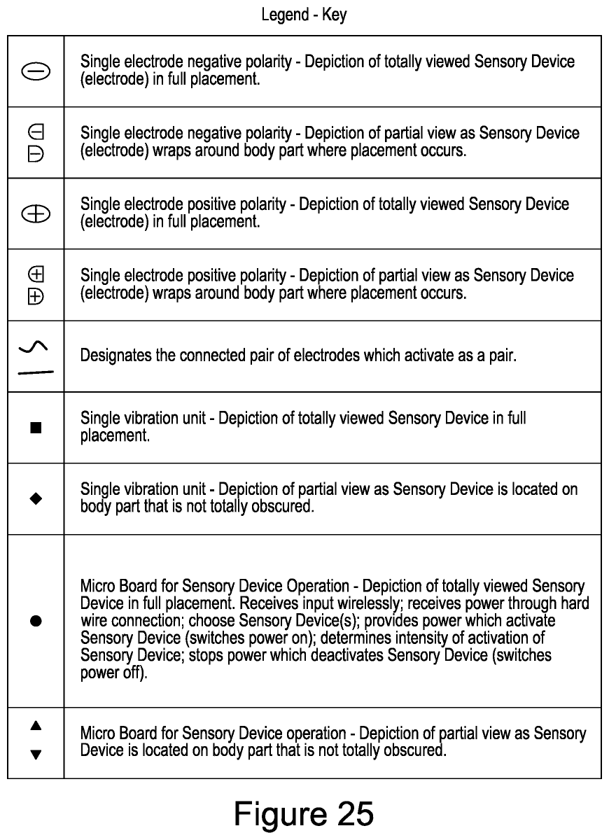

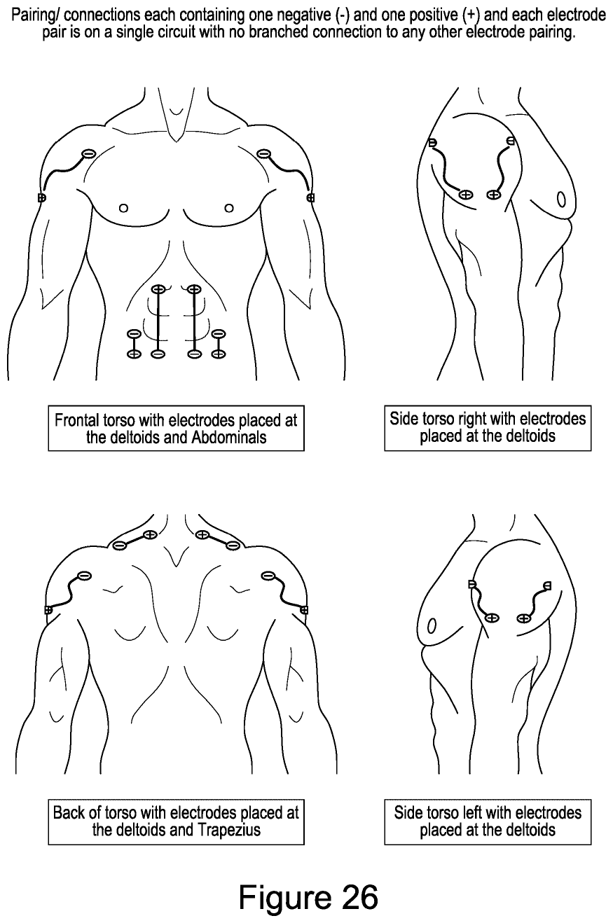

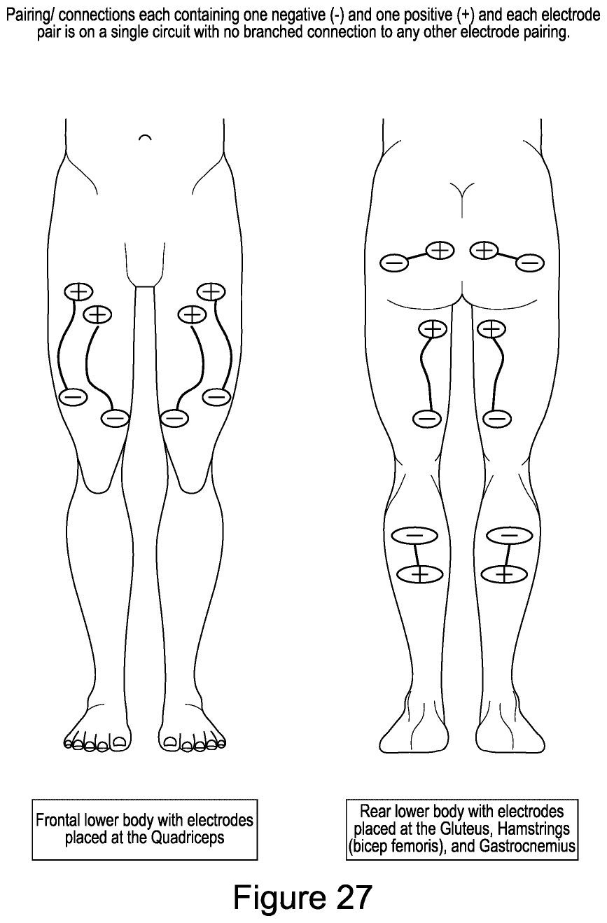

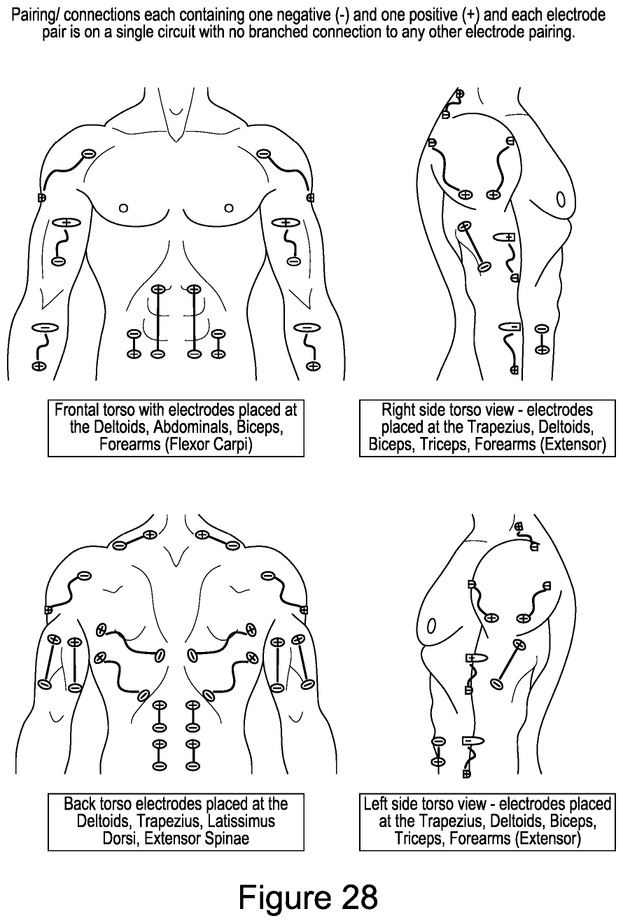

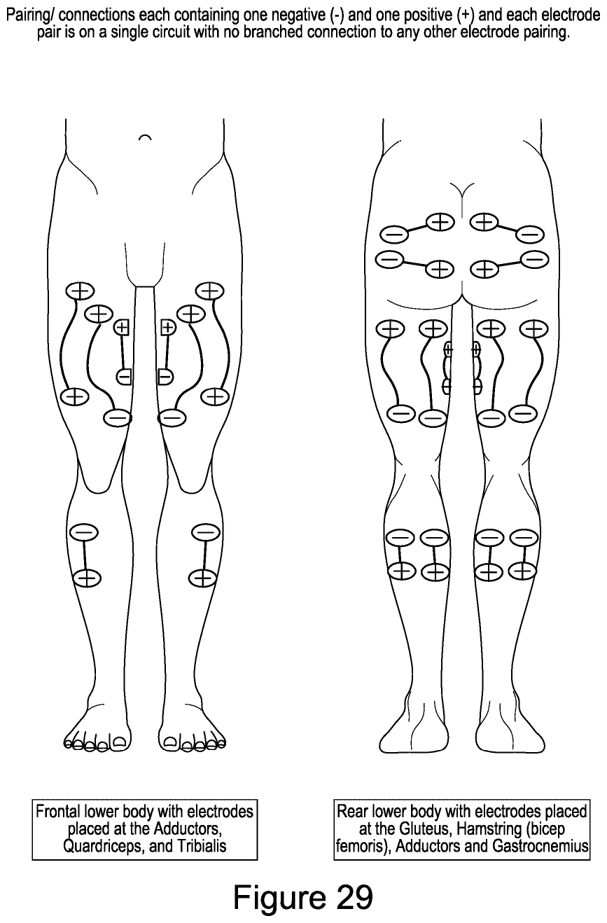

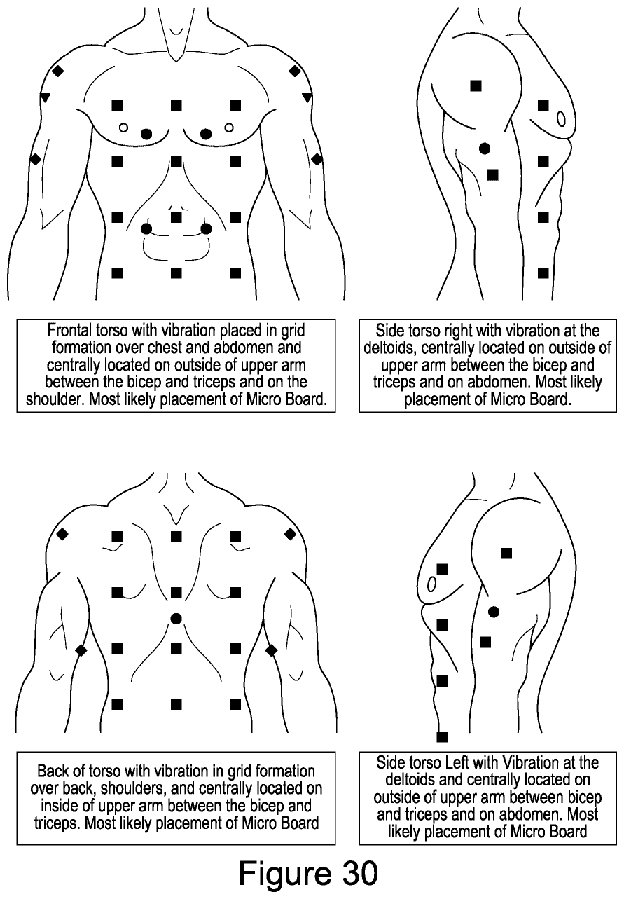

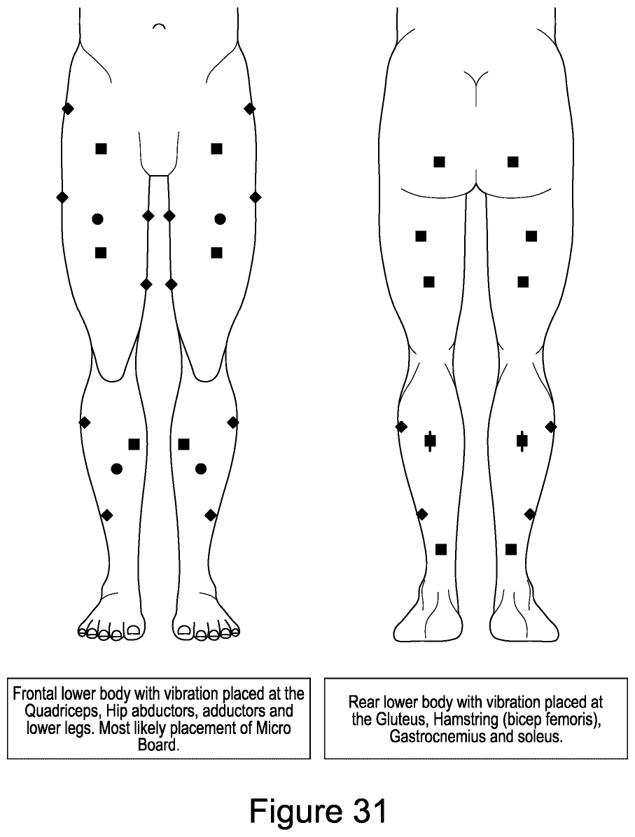

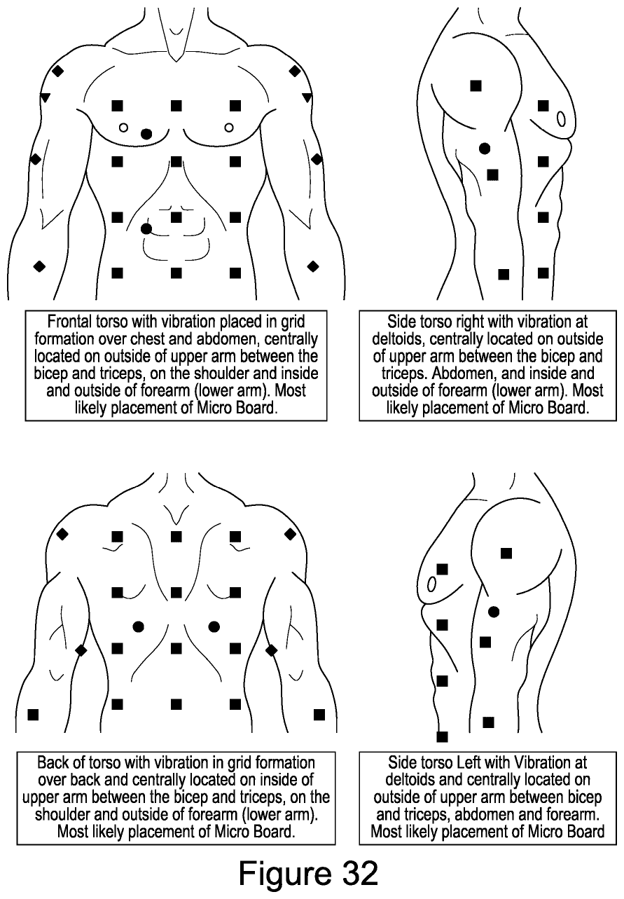

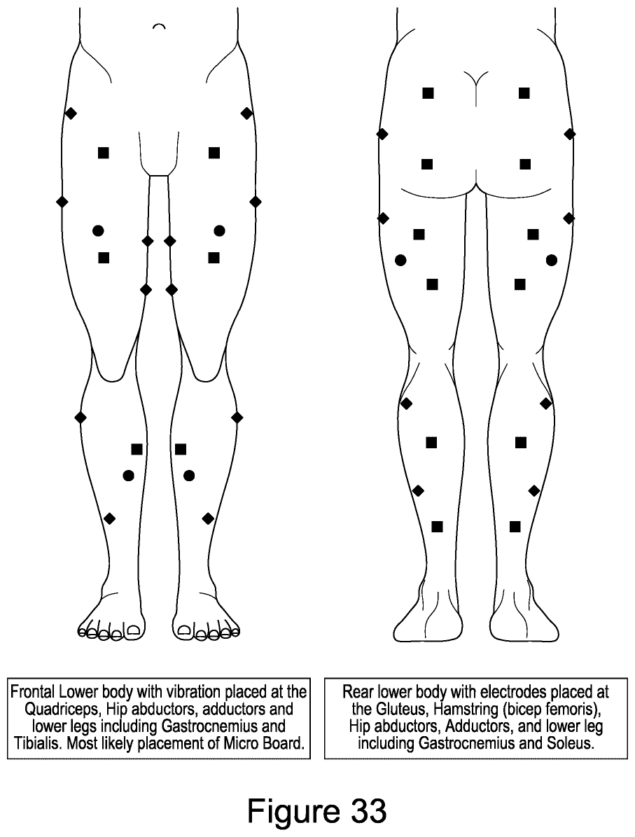

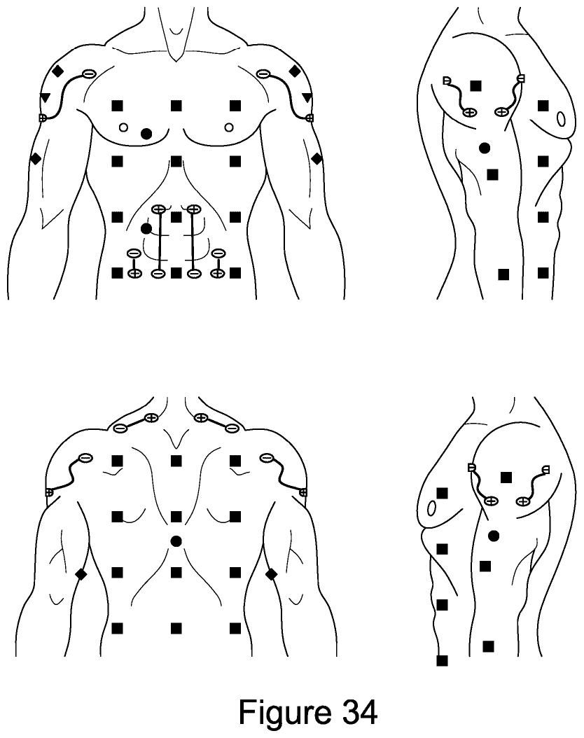

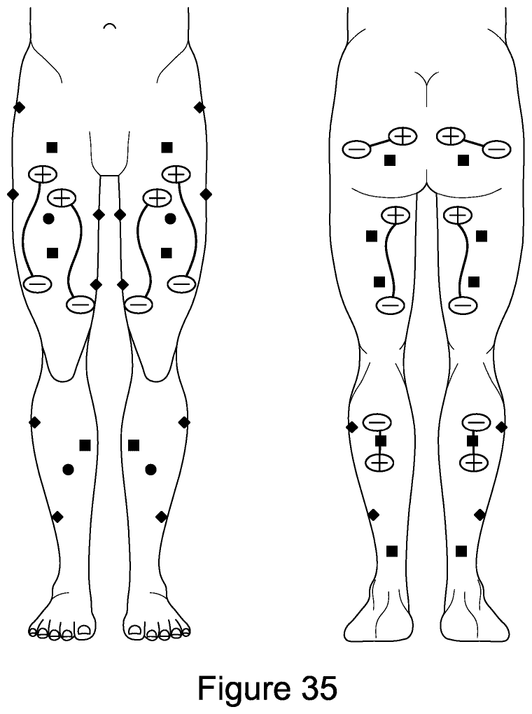

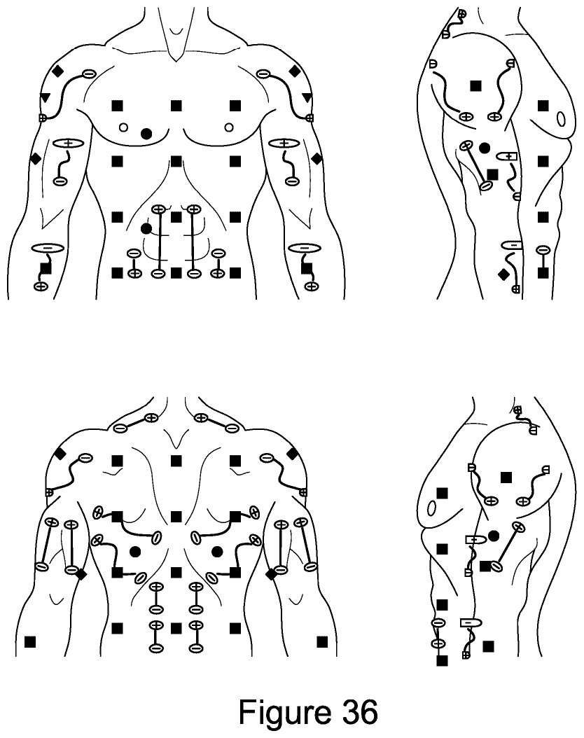

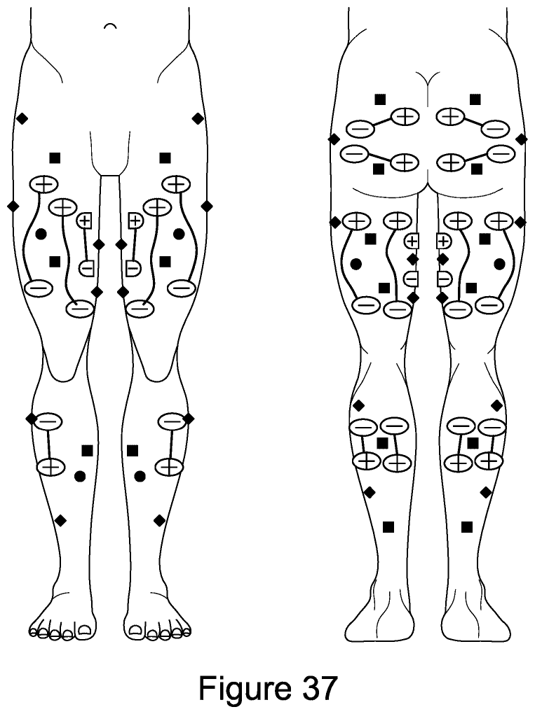

FIGS. 25 to 37 illustrate Sensory Device placement for example embodiments. FIG. 25 provides a legend for the symbols used in FIGS. 26 to 37. These are examples and other placements may be used for the Sensory Devices for various Sensory Stimulations. FIG. 26 provides examples of placements of the sensory devices on the user's body. Pairing/connections each containing one negative (−) and one positive (+) and each electrode pair is on a single circuit with no branched connection to any other electrode pairing. The top left provides a view of a frontal torso with electrodes placed at the deltoids and abdominals. The top right provides a view of a side torso right with electrodes placed at the deltoids. The bottom left provides a view of a back torso with electrodes placed at the deltoids and trapezius. The bottom right provides a view of a side torso left with electrodes placed at the deltoids. FIG. 27 provides examples of placements of the sensory devices on the user's body. Pairing/connections each containing one negative (−) and one positive (+) and each electrode pair is on a single circuit with no branched connection to any other electrode pairing. The left provides a view of a frontal lower body with electrodes placed at the quadriceps. The right provides a view of a rear lower body with electrodes placed at the gluteus, hamstrings (bicep femoris) and gastrocnemius. FIG. 28 provides examples of placements of the sensory devices on the user's body. Pairing/connections each containing one negative (−) and one positive (+) and each electrode pair is on a single circuit with no branched connection to any other electrode pairing. The top left provides a view of a frontal torso with electrodes placed at the deltoids, abdominals biceps and forearms (flexor carpi). The top right provides a view of a side torso right with electrodes placed at the trapezius, deltoids, biceps, triceps and forearms (extensor). The bottom left provides a view of a back torso with electrodes placed at the deltoids, trapezius, triceps, latissimus dorsi and extensor spinae. The bottom right provides a view of a side torso left with electrodes placed at the trapezius, deltoids, biceps, triceps, forearms (extensor). FIG. 29 provides examples of placements of the sensory devices on the user's body. Pairing/connections each containing one negative (−) and one positive (+) and each electrode pair is on a single circuit with no branched connection to any other electrode pairing. The left provides a view of a frontal lower body with electrodes placed at the adductors, quadriceps, and tibialis. The right provides a view of a rear lower body with electrodes placed at the gluteus, hamstrings (bicep femoris), adductors and gastrocnemius. FIG. 30 provides examples of placements of the sensory devices on the user's body. The top left provides a view of a frontal torso with vibration placed in a grid formation over chest and abdomen and centrally located on outside of upper arm between the bicep and triceps and on the shoulder. Most likely placement of micro board. The top right provides a view of a side torso right with vibration placed at the deltoids, centrally located on outside of upper arm between bicep and triceps and on abdomen. Most likely placement of micro board. The bottom left provides a view of a back torso with vibration in grid formation over back, shoulders and centrally located on inside of upper arm between bicep and triceps. Most likely placement of micro board. The bottom right provides a view of a side torso left with vibration placed at the deltoids and centrally located on outside of upper arm between bicep and triceps and on abdomen. Most likely placement of micro board. FIG. 31 provides examples of placements of the sensory devices on the user's body. The left provides a view of a frontal lower body with vibration placed at the quadriceps, hip abductors, adductors, and lower legs. Most likely placement of micro board. The right provides a view of a rear lower body with vibration placed at the gluteus, hamstrings (bicep femoris), gastrocnemius and soleus. FIG. 32 provides examples of placements of the sensory devices on the user's body. The top left provides a view of a frontal torso with vibration placed in a grid formation over chest and abdomen and centrally located on outside of upper arm between the bicep and triceps, on the shoulder, and inside and outside of forearm (lower arm). Most likely placement of micro board. The top right provides a view of a side torso right with vibration placed at the deltoids, centrally located on outside of upper arm between bicep and triceps, abdomen, and inside and outside of forearm (lower arm). Most likely placement of micro board. The bottom left provides a view of a back torso with vibration in grid formation over back and centrally located on inside of upper arm between bicep and triceps, on the shoulder and outside of forearm (lower arm). Most likely placement of micro board. The bottom right provides a view of a side torso left with vibration placed at the deltoids and centrally located on outside of upper arm between bicep and triceps, abdomen, and forearm. Most likely placement of micro board. FIG. 33 provides examples of placements of the sensory devices on the user's body. The left provides a view of a frontal lower body with vibration placed at the quadriceps, hip abductors, adductors, and lower legs including gastrocnemius and tibialis. Most likely placement of micro board. The right provides a view of a rear lower body with vibration placed at the gluteus, hamstrings (bicep femoris), hip abductors, adductors, and lower leg including gastrocnemius and soleus.

The application of this wearable technology as activated through a virtual medium or device, in that the virtual medium or device is what determines how the device interacts with the individual attached to the device, allows for consistency in Sensory Manipulation. Furthermore, this approach of the described technology is inventive as it allows virtual mediums to effectively create Sensory Outcomes based on real world Sensory Signatures using the virtual medium to enhance the effectiveness of that medium. In regard to a video game this would allow, but is not limited to, giving an individual the ability to have proper directional accuracy and a more localized and specific Sensory Stimulation to create a better Virtual Reality (VR) experience. For military this would allow, but is not limited to, a simulation having greater real-world quality as the synergistic actuation of multiple Sensory Devices such as EMS, Force, vibration, sound, and airflow create a simulation that cannot be reproduced elsewhere outside of real world activities. Such activities may include the effects of firing a gun, the character running with a heavy pack on their back, climbing, crawling and impacts of being shot and their locations on the body.

Usefulness of the embodiment shown herein may lie in various applications and fields of uses. Further, the multitude of market segment applications, its replicable outcomes and its association with a greater overall architecture provide additional use. The market segments include but are not limited to: entertainment industry, recreation industry, simulation training and medical rehabilitation. The replicable nature of stimulatory activations associated with the predetermined electrical stimulus interface device (electrodes 10) may allow for the consistency of expected future outcomes in each market application. One way this may be useful is in the video game market, for example, where software creators want their SDK protocols to evoke the same response on the player every time that specific protocol activates the device. The importance of repeatability in accuracy can easily be seen to extend to simulations and training, and medical rehabilitation which require outcomes to be consistent to ensure that the results are as expected to produce specific results.

Furthermore, individuals may be able to have a new, innovative enhanced and repeatable experience with a virtual medium that they were not capable of having before. Through the placement of the electrical stimulus interface (electrodes 10) individuals would be able to properly cover a great many locations of the body. Whether the technology is built into a garment 14 to allow the devices to cover the deltoids, abdominal, thigh, arm and various back muscles on an individual or the technology is built into any form of garment 14. The addition of the individualized local sound gives the individual using the device an immersive feel as they hear sounds as their avatar would. The addition of the Force Simulation Devices such as constriction/compression Stimulation Device actuators or Force/Physics Stimulation Device actuators gives additional sense of realism and is especially applicable in that the individual using the device physically feels the forces acting on them as their avatar does.

For various embodiments, the technology may be the same or similar and just the location of the hardware on an individual's body may be different depending on the particular tissue, nerve or muscles a virtual medium is designed to stimulate. Thus, through data sent by the computing device associated with the virtual medium it causes the WPEST technology to interact with the user through tissue, nerve or muscular stimulation that can create but is not limited to varying intensity, duration and radius of the body stimulated.

### Example 1

The following is an example of the wearable haptic navigation system directing users through various environments under various conditions.

An IOS application (“app”) for the iPhone 13 Pro was developed to facilitate the real-time tracking of users' positions within a given area. The app uses Apple's Augmented Reality Kit “ARKit 6” (https://developer.apple.comiaugmented-reality/arkiti) framework to build “Augmented Reality”(AR) experiences. These AR experiences require precise tracking of the phone's location in space alongside the precise tracking of a physical object's location. The wearable haptic navigation system takes advantage of these tracking properties to track a user equipped with an iPhone moving through an environment.

An Augmented Reality Session was created with Apple's ARWorldTracking-Configuration (https://developerapple.comidocumentationtarkitiarworldtrackingconfiguration) which: “tracks the device's movement with six degrees of freedom (6DOF): the three rotation axes (roll, pitch, and yaw), and three translation axes (movement in x, y, and z).” Every second, an ARAnchor (https://developer.apple.comidocumentationiarkitiaranchor) (An object that specifies the position and orientation of an item in the physical environment) is created and saved. These ARAnchors are bound to a real-world location, ensuring their stability as users move throughout the environment. This implementation creates a trail of “breadcrumbs” representing the user's path in an environment.

To enable data exchange, a transmission control protocol (TCP) server was implemented in the application, built using the SwiftNIO and NIOTrans-portServices libraries. This allowed a client to connect and access the “breadcrumb” data, alongside the user's current coordinates. This allowed the client to assemble the user's path and run the pathfinding algorithms on it to determine the optimal route for the user to navigate out of a building.

In this example, the A* search algorithm (https://en.wikipedia.orgiwiki/A*_search_algorithm) was used, giving exploration priority to nodes with favourable, in this case smaller, heuristic values. The A* search algorithm is a graph traversal and path search algorithm, which is used in many fields of computer science due to its completeness, optimality, and optimal efficiency. The A* algorithm only finds the shortest path from a specified source to a specified goal.

In this example, the Euclidean distance heuristic was used, which is the absolute distance between two points in 3-dimensional space. This was intuitive and the goals was to have firefighters travel the smallest distance possible as that is generally the quickest egress path for them.

The wearable haptic component in this example is the “As Real As It Gets” (ARAIG) haptic suit by IFTech Inc. as described herein. This haptic suit, which is worn as a t-shirt with two layers, provides vibratory feedback as well as direct muscle stimuli. The latter sensors, known as StimS, provide electrical stimulation directly to the surface of the skin. Over top of the StimS is the exoskeleton, which contains vibratory output sensors.

The StimS gave the users directions. One of the benefits of this system is it is as lightweight as possible. First responders, such as firefighters, carry immensely cumbersome gear, weighing anywhere from 20 to 30 kilograms, depending on what equipment they are carrying with them. A visual representation of the physical output on the suit can be seen in FIG. 39. The StimS makes the user feel being pulled in a specific direction. As best seen in FIG. 39, the wearable haptic component 2308 is shown from a front view and a rear view. Select haptic signal indicators 2314 are activated on the wearable haptic component 2308 to guide the user along the safe egress path.

The directions were output as follows, at a rate of once every 0.5 seconds, where α is the user's yaw relative to the next “breadcrumb” in his egress path:

Forward: π−½<α<π+½. The user's abdomen and pectorals are stimulated, indicating to them that they should move forward.

Left: π/2−½<α<π−½. The user's left shoulder is stimulated, indicating to them that they should turn left.

Right: π+½<α<3 π/2+½. The user's right shoulder is stimulated, indicating to them that they need to turn right.

Turn around: If a is not within the previous three ranges, the user is not facing the correct direction. Thus, the user's back is stimulated, prompting them to turn around.

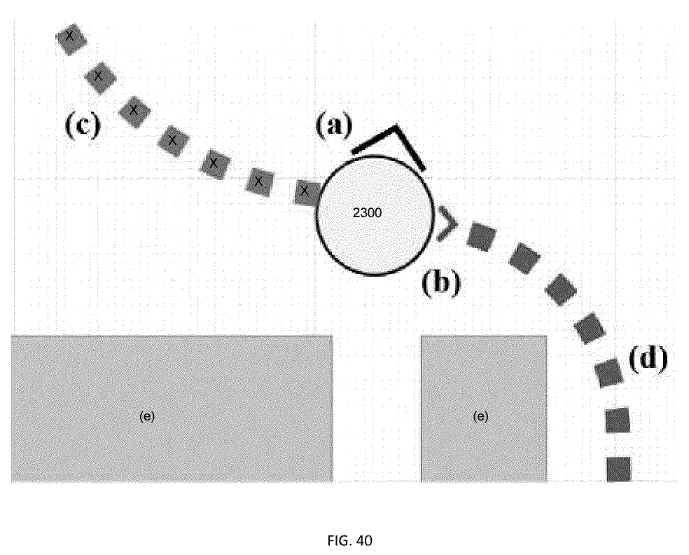

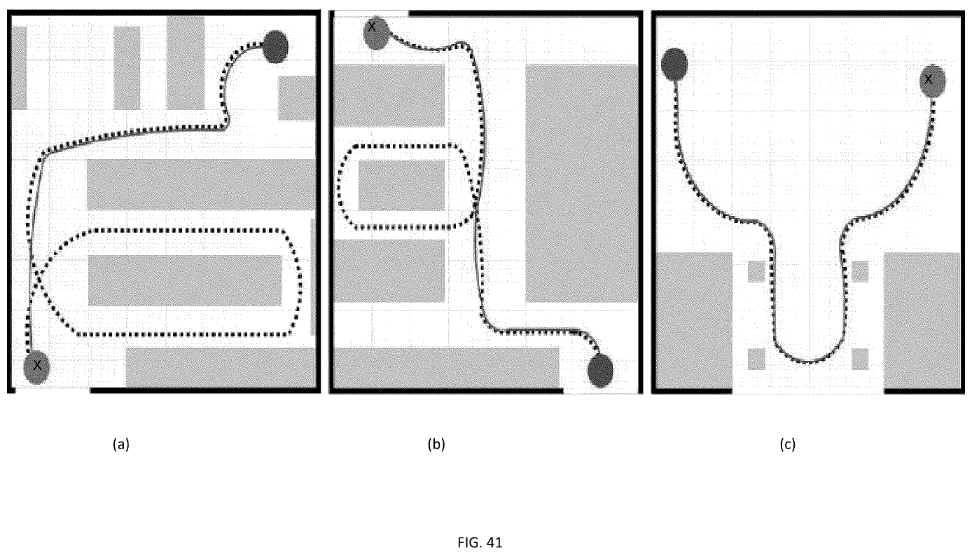

The design is meant to be intuitive with a very low learning curve. FIG. 40 demonstrates a top-down view of how the system navigates wearers to the exit, where the circle is the user 2300 from above; (a) is the direction the user is facing; (b) is the indicated directional output by the suit 2308; (c) is the path the user has already visited; (d) are the breadcrumbs which the user has not yet visited, and (e) are the obstacles in the room.

The example comprised three pathways:
a. a short path contained in a single room with multiple objects: FIG. 41(a);
b. a longer path across an entire floor of a multistory building with several minor obstacles: FIG. 41(b); and\
c. a long path outdoors, with few obstacles: FIG. 41(c).

These pathways can be seen in their correspondingly numbered figures, which includes dotted lines for the paths taken and solid lines for the most efficient egress pathway. Each pathway was subject to the three following tests:
a. no haptic feedback and no visual impairment;
b. with haptic feedback and no visual impairment; and
c. with haptic feedback and blindfolded.

To avoid building familiarity with the pathways, each subject was only tasked with one of the above tests for every pathway. For example, Subject B would explore the shortest path without the blindfold equipped and the suit on, whereas Subject C followed the path with the blindfold equipped in conjunction with the suit. Furthermore, the pathway from the entrance of a given area to the point where our subjects would need to start their egress paths was recorded previously, thus each subject would be completely unfamiliar with the path which the subject must follow. This was done to ensure a closer simulation of the disorientation a firefighter might feel in a low visibility, high chaos situation.

Every run was recorded on video, allowing us to time each experiment, and perform post analysis by reviewing each run in slow motion. It also allowed us to reference how easily each user interfaced with the suit. After each run, the wearer was asked a series of holistic questions, including: Did you find the physical directions intuitive?

Were you able to discern the directions being given to you or was there ambiguity?

Did you experience interruptions in the directional output?

What parts of the system would you suggest improving to make it more intuitive?

Do you see this system as being useful to firefighters? How about more general users?

Three subjects navigated the paths:
a. subject A: male, civilian, nearly completely unfamiliar with the project;
b. subject B: male, civilian, very familiar with the project; and
c. subject C: male, retired firefighter, some familiarity with the wearable haptic navigation system.

Table 1 shows the times taken for following the egress path from the point of indicated return to the “entry way” of the scenario, as shown in FIGS. 41(a)-(c) depicting not-to-scale recreations of the paths travelled and the egress paths recreated from the resulting point cloud. The plain circle is where the wearer begins, after the path has been recreated in dotted line, and he must follow the egress path, in solid line, to the corresponding exit circle with an “X”. The grey rectangles depict obstacles in the room. There were, in total, 28 attempted runs, 20 of which were in the long, indoor path across the floor plan of an old building, primarily comprised of concrete,

TABLE 1
Timed Results of Each Successful Run in Seconds (s)

| | | Haptic Assisted, | Haptic Assisted, |
|---|---|---|---|
| Path | Control | High Visibility | No Visibility |
| Short path, | Subject A: 15 s | Subject B: 29 s | Subject C: 140 s |
| single room | | | |
| Long path, | Subject B: 43 s | Subject C: 190 s | Subject A: 147 s |
| single structure | | | |
| floor plan | | | |
| Long path, | Subject B: 131 s | Subject C: 208 s | N/A |
| outdoors | | | |

Short Path, Single Room

The short path, single room trial faced little technical difficulties when tracking the path and communicating with the suit; there were few large physical obstructions. Rather, the room was full of smaller, superficial obstructions which may interrupt a pathway but not wireless communications. This is also the first area where we were able to test the suit with and without the blindfold.

Long Path, Single Structure Floor Plan

This map demonstrated the issues we anticipated with all forms of networking and communications, regardless of what we chose; interruptions in communications between devices were exceedingly common. While there were few superficial pathway obstructions, the walls of the old, multistory building we chose were made of thick concrete, thus causing interruptions in connectivity. That said, when the runs were successful, the speed through which blindfolded Subject A navigated the egress path was comparable with the time spent navigating by Subject C, who was not blindfolded.

After Subject C's egress path completion, it was decided by the researchers to lower the frequency of directional outputs from every 0.1 seconds to 0.5 seconds. Consequently, Subject A found the feedback far easier to follow in both his blindfolded test run in this location, as well as in the outdoor location.

Long Path, Outdoors

This run was primarily to test the viability of the system in an open area in outdoor conditions, thus the blindfolded aspect was omitted for this round of experiments.

The outdoor path tested both the distance capabilities of the system as well as if it could withstand basic outdoor weather. The distance interruptions seemed to happen at approximately 10 metres between devices, thus necessitating moving the network devices behind the suit wearer. That said, just as with the other maps, the suit's directions were able to navigate the user along the path and guide them to the exit. The weather did not appear to interfere at all with the system, though it was a cloudy summer day.

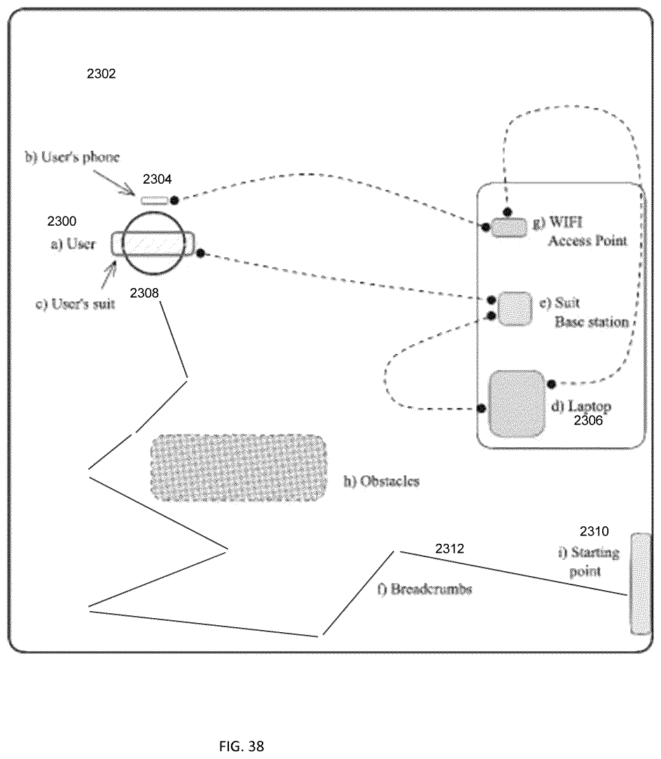

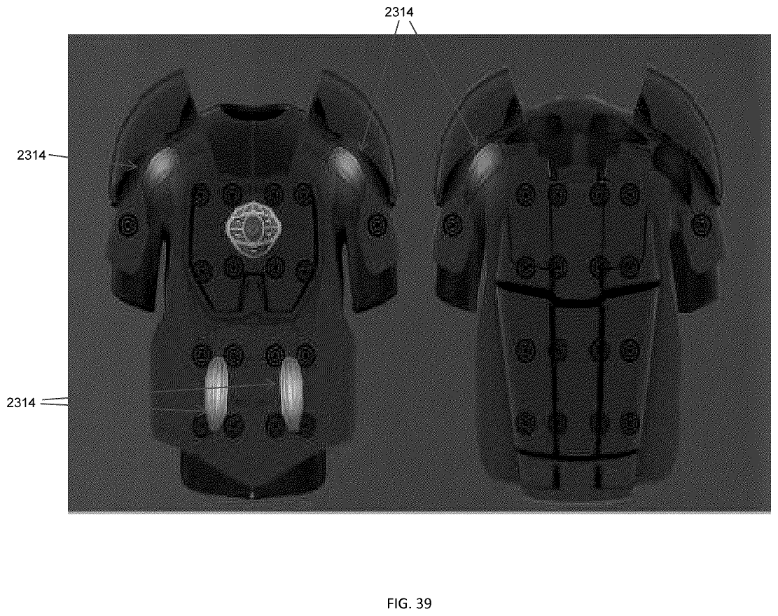

This test further demonstrated how the system navigates wearers back to points they already passed, should they choose a more optimal route with their vision and intuition. Subject A attempted to navigate back to the beginning of the egress path after accidentally circumventing all of the “breadcrumbs” set out for him. Referring now to FIG. 38, there is depicted a communication flow through the system 2302. The steps are as follows:
a. The user's phone 2304 “drops breadcrumbs 2312” as the user is navigating an environment;
b. When ready for the system 2302 to find an egress path, the program 2304 is started on a laptop 2306 which connects to the phone 2304 and requests the “breadcrumb” data;
c. The laptop 2306 runs the pathfinding algorithm(s) to find the optimal egress path;
d. The laptop 2306 requests the phone's location and calculates the relative angle to the user, given the user's yaw, to the next breadcrumb, then activates the corresponding stims; and
e. Repeats step 4 until the user has reached the starting position 2310.

Some characteristics of the wearable haptic navigation system include:
a. No prior spatial information is required (e.g. no maps, schematics, etc.,)
b. Support for passive augmentation of spatial information used to generate ad hoc maps
c. Support for multiple systems communication with each other to create and augment traversal maps providing near real-time traversability updates to each individual system supporting wayfinding within a SWS
d. Supports the generation and storage of the path travelled by a worker wearing the system
e. Supports calculation of shortest egress pathway(s) from a location occupied by a worker wearing the system, back to the original entry/starting point or alternate safe location as determined by interacting with other systems worn by other users
f Supports communication between users providing user identification (IDs) and determining user location and location relationship(s) to other users wearing the system
g. Supports monitoring and delivery of physiological data of the user
h. Supports monitoring and delivery of environmental data and
i. Supports navigation history of previous locations and points of interest of individual and multiple users through relationship management.

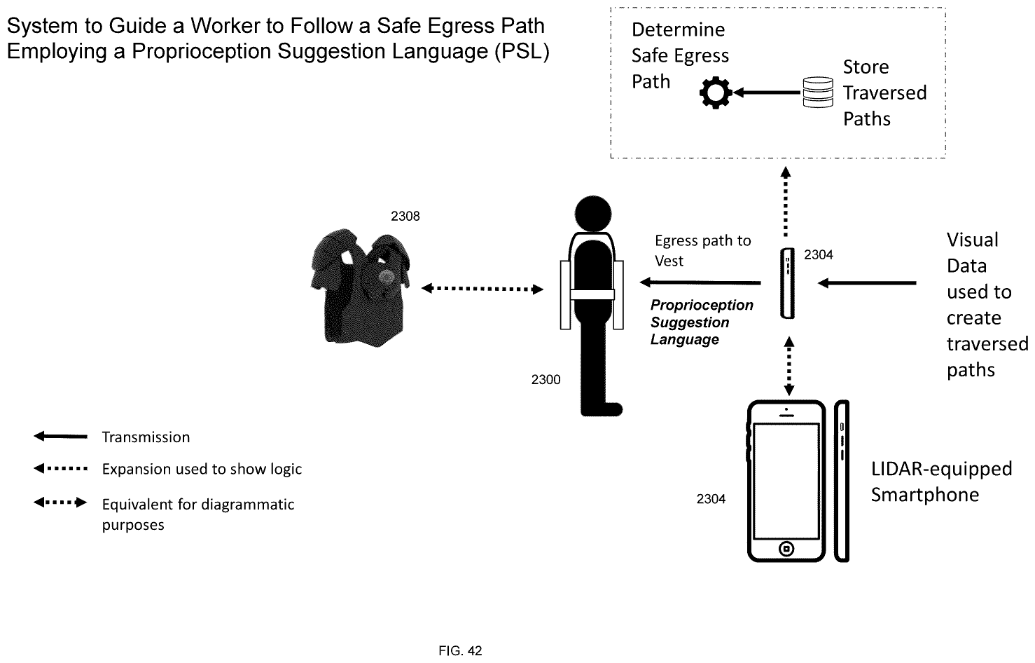

Referring now to FIG. 42, there is depicted how the wearable haptic navigation system works, in one alternative. The user 2300 is wearing the haptic component 2308, in this case the wearable suit as described herein, with a LIDAR equipped smartphone 2304 attached to the front of the wearable suit and collecting visual data to create and store data related to traversed paths. The LI DAR equipped smartphone 2304 calculates the safe egress path and transmits the data, in this case proprioception suggestion language (PSL), activating haptic signal generators on the haptic component 2308 guiding the user to follow a safe egress path.

Example 2 Notifications of upcoming movement(s) or object(s)

The wearable haptic navigation system, in one alternative, can urge or guide a user to move, stop, change direction, change body position (e.g., crouch, bend, twist, squat, raise arms, etc.,), and or change speed of movement (e.g., run faster or slower, walk faster or slower, crawl faster or slower).

Change body position or orientation.

In order to safely navigate it may be necessary to change the position of one's body and or orientation or slow down or speed up body movement. For example, a miner who is trying to get out of an area might need to crouch down and or crawl to get through a low-profile hole or tunnel or a firefighter who is crawling doing a primary search may need to immediately evacuate by standing and walking quicky or running and then slow down due to obstacles and speed up again or a police officer who is pinned down behind vehicles may need to crawl to a better location to allow for directional cues to return fire.

Bending down, bending forward, bending backward, twisting of the body

Sensors and or received data, mapping data collector and mapping data processor may indicate the necessity of bending down due to an obstruction, low headway, danger of being seen, harmful threat, etcetera. An example of one alternative is where the wearable haptic navigation system may urge, guide, or assist a user to bend forward. This may be initiated by the sensors and or received data, mapping data collector and mapping data processor indicating that there is a low beam and avoidance of that beam requires the individual to lower their head by bending forward. A micro processor takes the data using the proprioception suggestion language and sends signals to the wearable haptic component (or garment), the wearable haptic garment then responds to urge assist or guide the user to bend forward through for example electrical muscle stimulation on for example the rectus abdominus which contracts and shrinks the distance between the ribcage and the pelvis, or force feedback such as linear actuators on the back of the shoulders and neck as if someone is pushing you, or haptics using vibration or audio cues or any combination of electrical muscle stimulation, audio, haptic feedback, force feedback, constriction/compression, airflow, temperature stimulation.

Another example may include the indication of the user to bend sideways due to an overhead obstruction which has an opening to one side. This may be initiated by the sensors and or received data, mapping data collector and mapping data processor indicating that there is a low beam with clearance on one side and avoidance of that beam and clearance for traversing through the obstruction only requires the individual to bend to one side as necessary, linearly, diagonally, etcetera in order to pass the obstruction and continue on the path. The sensors and or received data, mapping data collector and mapping data processor indicate that there is a low beam with clearance on one side. The micro processor takes the data using the proprioception suggestion language and sends signals to the wearable haptic navigation garment, the wearable haptic navigation garment then responds to urge assist or guide the user to bend sideways, linearly, diagonally, etcetera. This assistance may occur through force feedback where a pushing feeling occurs on the left side shoulder and the use of electrical muscle stimulation occurs on the right side of the body on the obliques which contracts and shrinks the distance between the ribcage and the right hip or pelvis, or haptics using vibration or audio cues or any combination of electrical muscle stimulation, audio, haptic feedback, force feedback, constriction/compression, airflow, temperature stimulation. The use of any combined sensory stimulation may occur singularly, synchronous, intermittent, consecutive, imbricating and any combination thereof.

Twisting or turning to movement sideways.

Sensors and or received data, mapping data collector and mapping data processor may indicate the necessity of a user to twist sideways. This indication for the user to twist sideways may be due to a restriction in open space caused by a narrow space due to a building collapse, parked vehicles, land mines, etcetera. This may be initiated by the sensors and or received data, mapping data collector and mapping data processor indicating that there is a corridor of space that must be moved through which requires a thinner profile of the user. To create a thinner profile, it may be necessary to twist or turn the body in order to move through the limited space. The sensors and or received data, mapping data collector and mapping data processor indicate that there is a narrow space which the user may move through. The micro processor takes the data using the proprioception suggestion language and sends signals to the wearable haptic navigation garment, the wearable haptic navigation garment then responds to urge assist or guide the user to twist and or turn sideways. This assistance may occur through force feedback where a pushing feeling occurs on the left front side shoulder in combination with a force feedback pushing feeling on the right back shoulder to twist the upper torso or to turn the whole body sideways there may be through force feedback a pushing feeling occurring on the left front side shoulder and left front pelvis in combination with a force feedback pushing feeling on the right back shoulder and the right back hip or the use of through the use of haptics using vibration or audio cues or any combination of electrical muscle stimulation, audio, haptic feedback, force feedback, constriction/compression, airflow, temperature stimulation. The use of any combined sensory stimulation may occur singularly, synchronous, intermittent, consecutive, imbricating and any combination thereof.

Crouching Down

Sensors and or received data, mapping data collector and mapping data processor may indicate the necessity of a user to crouch down which generally consists of lowering the body stance by bending the knees which generally maintains the uprightness of the upper body but may also include bending over as previously described. As an example, the sensors and or received data, mapping data collector and mapping data processor may indicate the necessity of crouching down due to an obstruction, low headway, danger of being seen, harmful threat, etcetera. One alternative of how crouching may be achieved includes the sensors and or received data, mapping data collector and mapping data processor indicating that there is an area where the individual needs to crouch down which requires a lower profile of the user. To urge the user to crouch down, the sensors and or received data, mapping data collector and mapping data processor indicate that the user needs to crouch down and the micro processor takes the data using the proprioception suggestion language and sends signals to the wearable haptic navigation garment, the wearable haptic navigation garment then responds to urge, assist or guide the user to crouch down. This assistance may occur through force feedback where a pushing feeling occurs on top of both shoulders and neck in combination with EMS activations on the hamstrings and calves of both legs which contracts the muscles and shortens them urging the legs to bend. This could also include haptics to indicate a downward movement is required by using vibration to run down the body sequentially from the neck to the feet; the garment responds to the proprioception suggestion language to urge or assist the user to crouch through the use of any combination of haptics, audio, electrical muscle stimulation, force feedback, constriction/compression, airflow, or temperature stimulation. The use of any combined sensory stimulation may occur singularly, synchronous, intermittent, consecutive, imbricating (overlapping) and any combination thereof.

Crawling or Going Prone

Sensors and or received data, mapping data collector and mapping data processor may indicate the necessity of a user to get as low as possible and crawl on all four limbs or go prone and crawl close to the ground. In one alternative we may use a combination of methods from the bending over and crouching down to achieve urging a user to move to the crawling position. For example, the sensors and or received data, mapping data collector and mapping data processor indicate that crawling or getting down in the crawling position is now required, the sensors and or received data, mapping data collector and mapping data processor indicate that the user needs to crawl and the micro processor takes the data using the proprioception suggestion language and sends signals to the wearable haptic navigation garment, the wearable haptic navigation garment then responds to urge, assist or guide the user to crawl. This assistance may occur through force feedback where a pushing feeling occurs on top of both shoulders and neck in combination with EMS activations on the hamstrings and calves of both legs which contracts the muscles and shortens them urging the legs to bend. In addition, through electrical muscle stimulation on the rectus abdominus which contracts and shrinks the distance between the ribcage and the pelvis or force feedback such as linear actuators on the back of the shoulders and neck as if someone is pushing you, the garment responds to the proprioception suggestion language to urge or assist the user to crawl through the use of any combination of haptics, audio, electrical muscle stimulation, force feedback, constriction/compression, airflow, or temperature stimulation.

Another example may include the indication of the user to get as low as possible or go prone and crawl close to the ground. In one alternative we use the method of urging someone to move to a crawling position and we then add the extension of the arms. For example, the sensors and or received data, mapping data collector and mapping data processor indicate that prone positioning is now required, the sensors and or received data, mapping data collector and mapping data processor indicate that the user is in need of lying prone and the micro processor takes the data using the proprioception suggestion language and sends signals to the wearable haptic navigation garment, the wearable haptic navigation garment then responds to urge, assist or guide the user to the prone position. This assistance may occur through force feedback where a pushing feeling occurs on top of both shoulders and neck in combination with EMS activations on the hamstrings and calves of both legs which contracts the muscles and shortens them urging the legs to bend. In addition, through electrical muscle stimulation on the rectus abdominus which contracts and shrinks the distance between the ribcage and the pelvis or force feedback such as linear actuators on the back of the shoulders and neck as if someone is pushing you and EMS stimulation of the front shoulders and arms to assist in a movement that guides one to put their arms up or extended up past the head parallel to their torso, the garment responds to the PSL to urge or assist the user to crawl or go prone through the use of any combination of haptics, audio, electrical muscle stimulation, force feedback, constriction/compression, airflow, or temperature stimulation.

The use of any combined sensory stimulation may occur singularly, or in any combination synchronous, intermittent, consecutive or imbricating.

Walking or Running

Sensors and or received data, mapping data collector and mapping data processor may indicate the opportunity to walk or run, move fast or slow or speed up or slow down. There may be a need of personnel to speed up or slow down to move quickly in certain areas and more slowly in others as they traverse their environment. Although there may be obstacles such as walls, furniture, land mines, toxic environments, that affect the user such as fire fighters in large factories, vehicle showrooms, malls, etcetera; police pursuing a perpetrator; telerobotic navigator operating an unmanned ground/aerial or other vehicles, robots, etcetera; there may also be distance and time between objects or way points (change in position, direction, speed, etcetera as determined by the sensors and or received data, mapping data collector and mapping data processor) that that allow for a person to stand up and run, walk or speed up or slow down.

An example of one alternative is where the device may urge, guide, or assist a user to walk or run may be initiated by the sensors and or received data, mapping data collector and mapping data processor indicating that for a specified time, duration and or distance the user may walk or run. An example of how the system urges a user to walk or run may include where the sensors and or received data, mapping data collector and mapping data processor indicate the opportunity to walk or run and the micro processor takes the data using the proprioception suggestion language and sends signals to the wearable haptic navigation garment, the wearable haptic navigation garment then responds to urge, assist or guide the user to walk or run through audio cues, force feedback provided to the legs as well as haptic pulses indicating speed where a slow pulse would indicate walking and a fast pulse would indicate running and the modulation between the two indicates a change in speed such as slowing down or speeding up, the garment responds to the proprioception suggestion language to urge or assist the user to walk, run, speed up or slow down through the use of any combination of haptics, audio, electrical muscle stimulation, force feedback, constriction/compression, airflow, or temperature stimulation. The use of any combined sensory stimulation may occur singularly, or in any combination synchronous, intermittent, consecutive or imbricate.

Standing

It may be necessary to move someone from the crawling position to a standing position. Sensors and or received data, mapping data collector and mapping data processor may indicate the opportunity to move to a standing position from other positions. An example of one alternative is where the device may urge, guide, or assist a user to stand up may include the sensors and or received data, mapping data collector and mapping data processor indicating that there is a requirement to stand up. The micro processor takes the data using the proprioception suggestion language and sends signals to the wearable haptic navigation garment, the wearable haptic navigation garment then responds to urge, assist or guide the user to stand up. This may be accomplished through constriction on the lower leg for a feeling of not moving the feet, EMS on the thighs to urge the straightening of the legs, force feedback used on the torso with vibration moving up the body to give a lifting sensation, the garment responds to the PSL to urge or assist the user to stand through the use of any combination of haptics, audio, electrical muscle stimulation, force feedback, constriction/compression, airflow, or temperature stimulation.

Jumping

It may be necessary to know when to jump over something such as when encountering a hole, dangerous substance, trip wire, landmine, etcetera. Sensors and or received data, mapping data collector and mapping data processor may indicate an area where jumping is required. An example of one alternative is where the device may urge, guide, or assist a user to jump over something where the sensors and or received data, mapping data collector and mapping data processor indicate that there is a requirement to jump. The micro processor takes the data using the proprioception suggestion language and sends signals to the wearable haptic navigation garment, the wearable haptic navigation garment then responds to urge, assist or guide the user to jump. This may be accomplished through lead up and burst emission to specific areas of the body. This burst emission may consist of audio cues, EMS to the thighs and glutes (which are used to jump) while the lead up may consist of audio cues and haptic instructions. The lead up may consist of audio cues and low intensity haptics which get stronger and then conclude with a burst emission of EMS on the thigh and gluteus muscles; the garment responds to the PSL to urge or assist the user to jump through the use of any combination of haptics, audio, electrical muscle stimulation, force feedback, constriction/compression, airflow, or temperature stimulation. The use of any combined sensory stimulation may occur singularly, synchronous, intermittent, consecutive, imbricating and any combination thereof.

Orientation

It may be necessary to know your body position relative to your surroundings such as which way is up or which way is down as may occur to an air pilot when the air pilot goes into a rapid roll where they become disoriented due to the continued feeling of leaning caused by the initial inertia of the roll even after the motion has stopped. Or someone in a confined space who loses their point of reference. Sensors and or received data, mapping data collector and mapping data processor may indicate when the user is disoriented, and action is required. An example of one alternative is where the device may urge, guide, or assist a user to roll a plane/unmanned aerial vehicle (UAV) right-side up from upside down. This may include where the sensors and or received data, mapping data collector and mapping data processor indicate that there is a requirement to right the plane. The micro processor takes the data using the proprioception suggestion language and sends signals to the wearable haptic navigation garment, the wearable haptic navigation garment then responds to urge, assist or guide the user to take the correct actions to right the plane. This may be accomplished through haptic moving across the body consecutively from low intensity increasing intensity as they activate across the body where it starts at one side of the body and finishes on the side where the turn should be made (roll to the left then the low intensity starts on the rights side and increases till it hits the left side). This is combined with EMS which urges the arms and shoulders to steer to the left; the garment responds to the PSL to urge or assist the user to reorient (ex. through steering the plane) through the use of any combination of haptics, audio, electrical muscle stimulation, force feedback, constriction/compression, airflow, or temperature stimulation. The use of any combined sensory stimulation may occur singularly, synchronous, intermittent, consecutive, imbricating and any combination thereof.

Attraction/Pulling

There may be a need for a greater force to be used to initiate the movement of an individual or to be applied at times of stress when greater or stronger cues are required to initiate or continue actions of an individual. For example, a police officer is under fire and the system has positioned him to crawl. Once in that position and under fire he may need to be initially pulled in a direction to start the crawling process. In one alternative, the feeling of being pulled in a specific direction can be achieved through sensory stimulation using vibration which starts from the periphery of the body on equal sides of the directional cue and pulses inward sequentially toward the object or direction and then moves directly back to the periphery and again pulses inward sequentially toward the object or direction combined with EMS or TENS (transcutaneous electrical nerve stimulation) activations directly on the aligned area of the body in the direction to which they are to face or move; the wearable haptic navigation garment responds to the PSL to attract or pull the user toward a direction, object person place or thing the use of any combination of haptics, audio, electrical muscle stimulation, force feedback, constriction/compression, airflow, or temperature stimulation The use of any combined sensory stimulation may occur singularly, synchronous, intermittent, consecutive, imbricating and any combination thereof.

Repulsing/Pushing

There may be a need for a greater force to be used to stop the movement of an individual or to be applied at times of stress when greater or stronger cues are required to stop the movement or continued actions of an individual. For example, a police officer is under fire, and he decides to stand up and run in a certain direction that the system has deemed dangerous, e.g. running into line of fire. It may be necessary in this situation to stop their movement by pushing or repelling them from the direction they are going or the movement they are performing. In one alternative, the feeling of being pushed can be achieved through the combination of constriction/compression to both the upper torso and lower body while using haptic bursts to get their attention. In another alternative repulsion may be achieved through force feedback applied in opposition to the movement of the individual combined with EMS stimulations to the legs and or arms to counteract the movement; the wearable haptic navigation garment responds to the PSL to push or repulse the user away from an object, person, place or thing through the use of any combination of haptics, audio, electrical muscle stimulation, force feedback, constriction/compression, airflow, or temperature stimulation. The use of any combined sensory stimulation may occur singularly, synchronous, intermittent, consecutive, imbricating and any combination thereof.

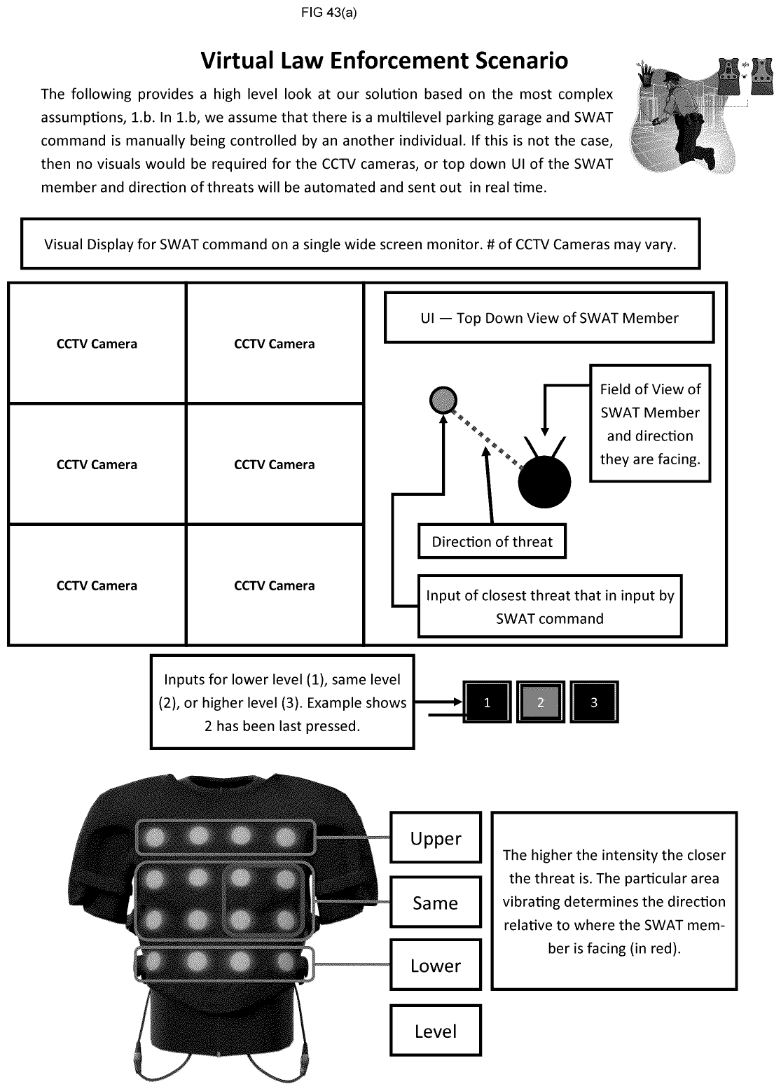

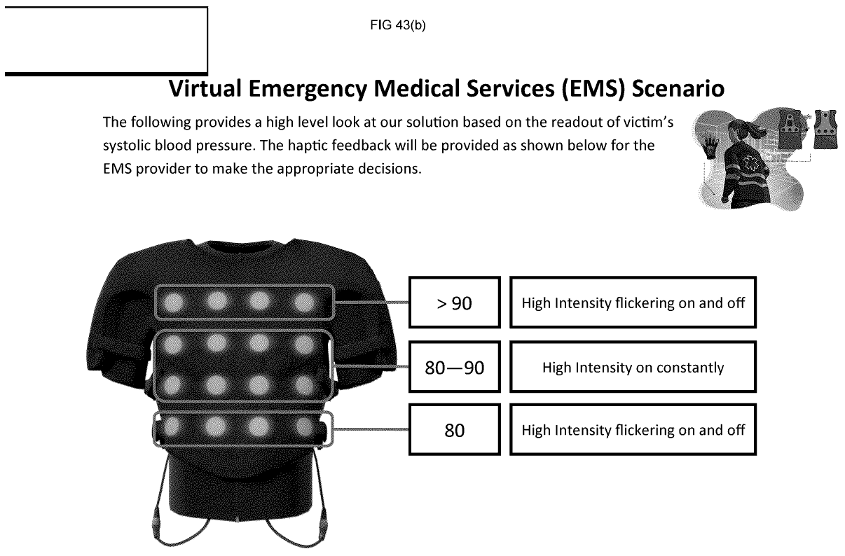

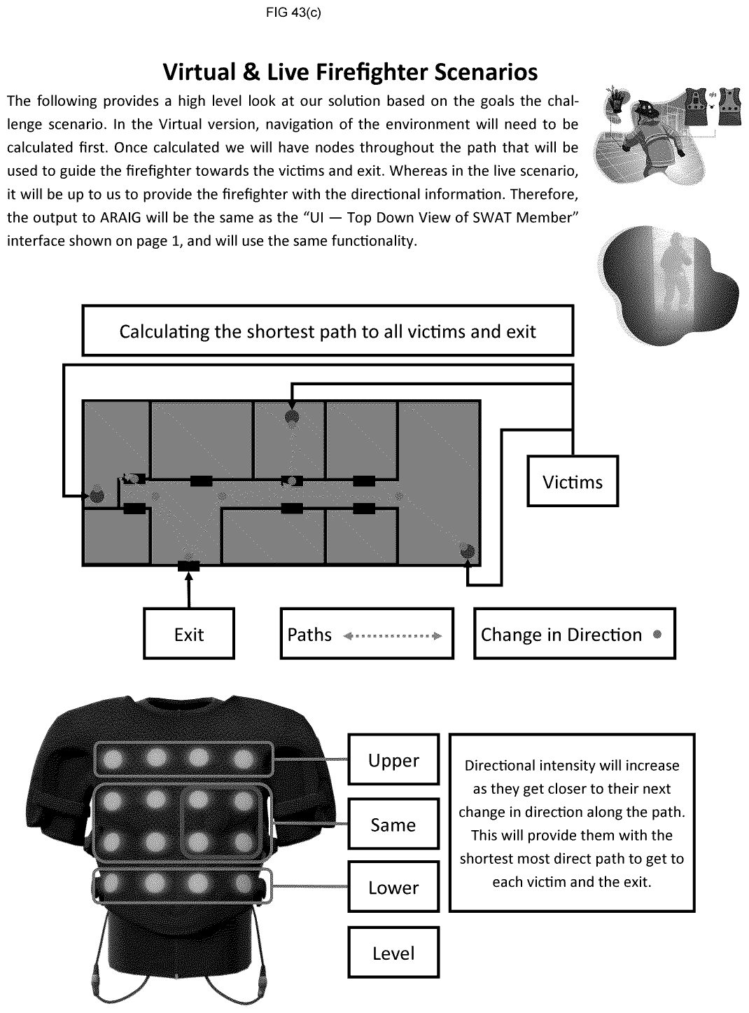

Example 3 Law Enforcement Scenario (see FIG. 43(a))

A software program was created by the National Institute of Standards and Technology (NIST). The program was designed specifically for users wearing a head mounted display. The software program was a synthetic environment designed to test haptic direction for a police officer pinned down behind a parking barricade and can not determine direction of the perpetrator(s) or from where the gunshots are coming. The wearable haptic navigation system provided haptic directional cues to aim the police officer and their weapon directly at the assailant. The haptic cues for this scenario used periphery to center vibration activations to guide the user from their original position/direction to pointing the center of their chest at the direct location of the assailant. All users of the system were able to easily reposition (change direction) themselves to direct return fire at the assailant.

Example 4 Virtual Firefighter Scenario (See FIG. 43(c))

A software program was created by the National Institute of Standards and Technology (NIST). The program was designed specifically for users wearing a head mounted display. The software program was a synthetic environment designed to test the ability of haptics to direct users to a location under smoky environments. The purpose was to get visibly limited personnel from point A to point B faster than currently possible using traditional methods of feeling walls, crawling on floors, etcetera. Using the same methodology for the law enforcement scenario above, haptic cues using periphery to center vibration activations to guide the user from their original position/direction to pointing the center of their chest in the direction that they should be moving. All users of the system were able to easily travel the blacked-out maze within seconds.

Example 5 Virtual Emergency Medical Services (EMS) Scenario (See FIG. 43(b))

A software program was created by the National Institute of Standards and Technology (NIST). The program was designed specifically for users wearing a head mounted display. The software program was a synthetic environment designed to test the ability of haptics to monitor and then inform a single user about patients in a multicausality incident. The purpose was to ensure that any patient who was attended to, and deemed viable would be monitored and in the case that their vitals indicated a problem the user would immediately be informed of the particular patient and the level of the vitals (systolic pressures) that were being indicated as problematic. In addition, the user could choose a particular patient at any time to check on the vitals. The wearable haptic navigation system provided the user with a) the ability to connect the patient to the list of monitored personnel which were to be monitored by the wearable haptic navigation system, b) the ability to receive information on blood pressure of any given patient chosen, and d) identification of and immediate information on a specific patient when the vitals of that patient are at a dangerous level. In the synthetic environment all users were able to hook all patients up to the system and check on any patient at any time to check on their vitals. In addition, all users responded appropriately to finding the correct patient when the blood pressure dropped dangerously low and the haptics information was provided to them.

Example 6 flight simulator with the wearable haptic navigation system

The type of forces and effects an air pilot experiences during a real flight need to be reproduced, by combining a sensory stimulation flight simulator interface with the wearable haptic navigation system, we can (a) recreate the forces and effects an air pilot experiences during a real flight or (b) create some stimulus that causes the same physical effects (i.e. increase in blood pressure/hate rate).

The wearable haptic navigation system receives a signal from a flight simulation interface providing the necessary feedback to the user of the wearable haptic navigation system replicating the type of forces and effects an air pilot experiences during a real flight. These forces and effects are created by actions of the plane or air pilot and may include but are not limited to: wind shears; updrafts; downdrafts; stalls; engine failure/flame out; pitch attitude greater than 25° nose up; pitch attitude greater than 10° nose down; bank angle greater than 45°; airspeeds inappropriate for the condition; turbulence; g-force; constriction; pressure; falling; etcetera.

Flight simulation is configured to google maps, VBS3 simulations or other geolocation live streaming software providing real world geographical, territorial, topographic, time of day and weather for flight simulations.

The combination of the wearable haptic navigation system with the flight simulator results in a flight simulation technology (both hardware and software) for upset prevention and recovery training and unmanned aerial vehicle (UAV) operations.

The design is modular based to allow for additional functionality to be added with limited risk of damaging previously validation functionality. This modular design allows for:
a. interface development with other flight simulation devices on the market;
b. development/integration of new simulated effects, evaluation and AI algorithms;
c. all devices talk via different interfaces;
d. a sensory stimulation flight simulator interface shall provide the ability to connect via the internet, as well as, provide the ability to add any type of data communications medium (e.g., ARINC, MIL-STD-1553, etc.); and
e. support the display and collection of telemetry and system events in real-time to allow for monitoring, debugging, analysis and evaluation of the algorithms and new technology being tested. The sensory stimulation flight simulator interface software is designed to support replay capabilities in case a real-time simulated device is not connected to the system.

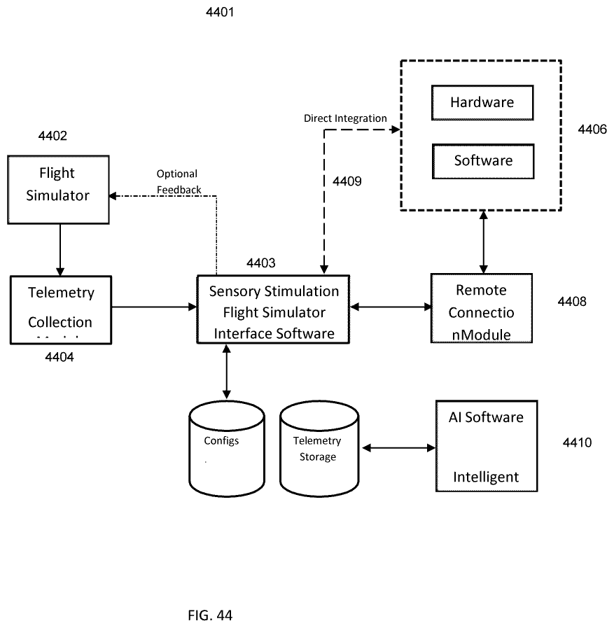

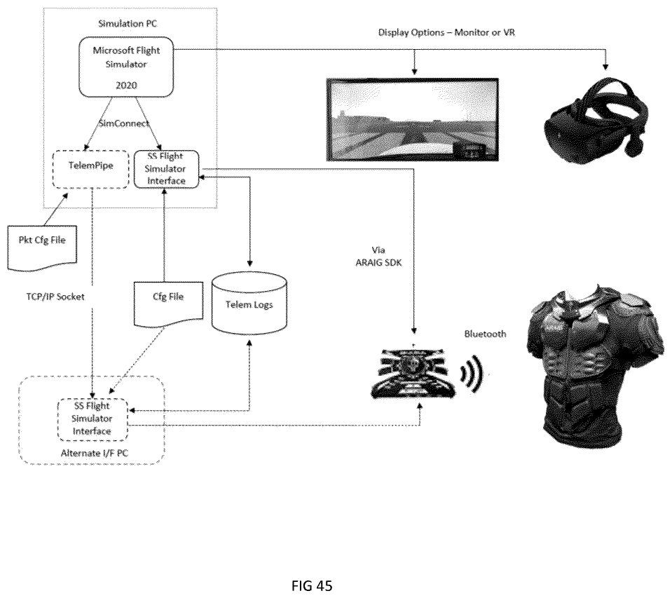

Referring now to FIG. 44, there is shown a system level block diagram for the ARIAG flight training system. The system monitors flight parameters in real-time and converts the telemetry data into haptic and vibratory responses for ARIAG suit. The system includes a sensory stimulation flight simulator interface 4401 including a flight simulator 4402, a telemetry collection module 4404, a 3rd party technology module 4406, a remote connection module 4408 and an AI software/intelligent processor 4410.

The flight simulator 4402 represents a 3rd party simulation training device connected to the sensory stimulation flight simulator interface system 4400. The system 4400 is designed to interface generically with any flight training device on the market. Once a decision is made to connect a unit, an interface module will be created using the telemetry collection module definition.

Sensory stimulation flight simulator interface software works based on a standard definition of telemetry. The Telemetry Collection Module 4404 provides the definition and interface to this generic structure. It allows for flight simulation telemetry to be collected and stored for later analysis and/or injection into virtual reality/augmented reality training simulation modules or processed in real-time. It further allows for a protocol conversion between the flight training device and the sensory stimulation flight simulator interface software 4403, allowing the sensory stimulation flight simulator interface processing to stay generic internally.

The sensory stimulation flight simulator interface software 4403 is designed to select the interface protocol based on the name of the simulation configuration file. This means the integration of a new flight simulation device requires only a new module and simple update to the sensory stimulation flight simulator interface software 4403 to recognize the new configuration filename. This processes the flight simulator telemetry and converts in into haptic and vibratory inputs for the ARAIG suit. Processing of the telemetry is performed using predefined configuration files that may be tailored to specific missions, flight training modules and/or aircraft type.

The sensory stimulation flight simulator interface provides a high-level menu to access all the different functionality and configuration capabilities of the system. Sensory stimulation flight simulator interface 4400 is also designed to be an evaluation and research tool. The 3rd Party Technology box 4406 shows how medical and gaming technology may be connected to the system. There are two options:

Direct Integration 4409— The integration of the technology directly into the Sensory Stimulation Flight Simulator Interface software via a Software Development Kit (SDK) or Application Program Interface (API).

Remote Connection 4408— The software/hardware is connected using a specified protocol and hardware medium, such as internet sockets, serial, ARINC, MIL-STD-1553, etc.

The remote connection module 4408 provides the configurable ability to select and communicate with external technology, such as the ARIAG suit via a separate physical medium. Initial releases will provide the ability to configure and connect to other hardware/software using internet-based sockets (TCP/IP or UDP/IP). As new interfaces are required, such as serial, ARINC, MIL-STD-1553, new remote connection modules can be created to support, configure and communicate via these interfaces.

The AI software/intelligent processing software system 4410 has been designed to support the development of smart artificially intelligent evaluation tools to help trend/predict behavioral patterns in student pilots. In this alternative, the software is remote from the sensory stimulation flight simulator interface/flight simulation device. Therefore, the design of the sensory stimulation flight simulator interface software is to provide a default remote connection using Internet based protocols for this capability.

Referring now to FIG. 45, there is provided another alternative of a flight simulation system in communication with the wearable haptic navigation system. It further shows two configurations for the system depicted in FIG. 44.

It depicts that the simulator can be run in Desktop (the monitor) or virtual reality mode (the headset) with ARAIG attached. There are two methods of connection for the ARAIG system. If you have a powerful PC running the simulator, and have resources left over, you can run the flight simulator interface (SimLE) on the same PC as the simulator software. The ARAIG suit then communicates directly with the simulator PC.

If your simulator PC is lacking resources and needs all the CPU processing power for running the flight simulator itself, you can remotely connect ARAIG through a secondary system. In this situation SimLE is running on a separate computer with an ethernet TCP/IP socket connection. The simulator telemetry is packetized and transmitted via the telemetry pipe to the secondary PC. The secondary PC (and its resources) then process the simulator telemetry and command the ARAIG suit

The wearable haptic navigation system introduces realistic human factors inside flight simulation training devices. The use of wearable haptic navigation system will help simulate Gx and Gy forces on the pilots body based on acceleration and forces calculated within the simulated environment. Using the wearable haptic navigation system to increase heart rate and blood pressure will also allow for better upset prevention and recovery training (UPRT) scenarios. Placing a pilot in a condition where their critical thinking skills and response times are reduced.

Joined with medical monitoring devices the system will collect and learn what an “ideal” pilot would look like. Eventually providing the ability to trend or predict behavioral patterns in pilot students. Data that could be used for both military selection, astronaut selection and even student pilot retention.

This system reduces the carbon footprint of traditional flight training by having trainees spend more time on wearable haptic navigation system upgraded simulators instead of flying in the air.

This not only helps in preventing injuries to trainees and/or others but reduces damage/loss of equipment.

And as downtime is also a problem, less airtime training puts less mileage on vehicles and reduces servicing/maintenance.

According to another aspect, there is provided a wearable device comprising:
a. a wearable garment;
b. an input module to collect sensory related data;
c. a plurality of sensory devices connected to the wearable garment that actuate to produce one or more sensory stimulations, each of said one or more sensory stimulations for inducing physiological stimulation; and d. a control centre comprising:
e. a processor for determining sensory events, each of said sensory events defining a synergistic action of said one or more sensory stimulations as a signal pathway to produce one or more sensory outcomes, each of said one or more sensory outcomes for inducing a physiological response or sensory perception;
f. a transceiver for receiving the sensory related data collected via the input module, and in response, sending an activating signal to actuate one or more of said plurality of sensory devices to activate the sensory events wherein the synergistic action of two or more sensory stimulations comprise at least two of electrical muscle stimulation, audio, haptic feedback, force feedback, constriction/compression, airflow, temperature stimulation and combinations thereof; said wearable device in communication with a flight simulator.

In general, some benefits include using the mapping data, the relationship data, the sensor data, the incoming communication data, manual input data, etcetera, will determine the body position of the user and/or safe path and then suggest to the user through sensory stimulation the body position and/or direction to move into or directional path to follow.

Elapsed time for following egress paths was greater wearing the suit even when there were no visibility issues. Wearing the suit always increased the timeframe for traversing the workspace.

1. A wearable haptic navigation system assisting a user through obscured visibility environments, said wearable haptic navigation system comprising:
a. a wearable haptic component; and
b. a mapping data collector and mapping data processor in communication with said wearable haptic component, wherein said wearable haptic component comprises a wearable device comprising:
a wearable garment
an input module to collect sensory related data;
a plurality of sensory devices connected to the wearable garment that actuate to produce one or more sensory stimulations, each of said one or more sensory stimulations for inducing physiological stimulation; and
a control centre comprising:
a processor for determining sensory events, each of said sensory events defining a synergistic action of said one or more sensory stimulations as a signal pathway to produce one or more sensory outcomes, each of said one or more sensory outcomes inducing a physiological response or sensory perception;
a transceiver receiving the sensory related data collected via the input module, and in response, sending an activating signal to actuate one or more of said plurality of sensory devices to activate the sensory events wherein the synergistic action of two or more sensory stimulations comprise at least two of electrical muscle stimulation, audio, haptic feedback, force feedback, constriction/compression, airflow, temperature stimulation and combinations thereof; wherein said mapping data collector i) collects data, including visual data, of a path travelled by said user in said obscured visibility environments and/or ii) retrieves pre-existing mapping data, including visual data, and said mapping data processor calculates from at least one of the collected mapping data and/or the retrieved pre-existing mapping data, i) a safe path for said user from a first point to a second point and ii) sends haptic signals to said wearable haptic component via proprioceptive suggestion language suggesting said safe path to said user and suggesting at least one of a safe body position, direction and speed of travel to said user.

2. The wearable haptic navigation system of claim 1, wherein said mapping data collector is selected from the group consisting of
a. LiDAR;
b. Radar;
c. Sonar;
d. Camera;
e. environmental sensor;
f. high-definition(HD) map;
g. Inertial sensor;
h. Echosounder;
i. visible light;
j. Ultra-wideband;
k. Ultrasonic;
Pseudolite;
Wireless fidelity;
n. Bluetooth low energy;
o. Visual Simultaneous Localization and Mapping (vSLAM);
P. Infrared;
q. Thermal;
r. Low Frequency Magnetic Waves;
s. communication system including global navigation satellite system (GNSS), assist in mapping, positioning, localization and navigation which help to determine distance speed, positioning and route guidance; and combinations thereof.

3. The wearable haptic navigation system of claim 2 wherein said mapping data collector is a personal two-way communication device.

4. The wearable haptic navigation system of claim 1, wherein said mapping data collector, said mapping data processor and said wearable haptic component are in with each other via a wired, wireless and combinations thereof communication system.

5. The wearable haptic navigation system of claim 4, wherein said wireless communication system is selected from the group consisting of Bluetooth™, Wifi, radio, satellite, mobile, wireless network, infrared, microwave, GPS, ZigBee and combinations thereof.

6. The wearable haptic navigation system of claim 1, wherein said mapping data collector and said mapping data processor further collects data of a local environment proximate said user creating a map of known wayfinding points and sends said known wayfinding points to the wearable haptic component directing the user to an egress location and/or point.

7. The wearable haptic navigation system of claim 1, wherein said wearable haptic navigation system circumvents localization issues associated with Global Positioning Systems (GPS).

8. The wearable haptic navigation system of claim 6, wherein said wearable haptic navigation system provides physical directions to said user in a continuous direction output.

9. A method of guiding a visibly challenged user and/or a user in an environment with obscured visibility, along a safe egress path and/or in a safe body position, said method comprising the use of the wearable haptic navigation system of claim 1.

10. The method of claim 9 further comprising:
a. collecting data, in one alternative visual data of a path traversed by a user, said data collected by a mapping data collector equipped device;
b. creating a traversed path from the data collected by a mapping data processor;
c. storing the traversed path;
d. determining a safe egress path and/or safe body position from the stored traversed path; and
e. communicating the safe egress path and/or safe body position to a wearable haptic component worn by said user, by proprioceptive suggestive language translated to haptic signals on the wearable haptic component urging the user to the safe egress path and/or safe body position.

11. The method of claim 10, wherein said haptic signals comprise directional commands, safe body position commands, velocity commands and combinations thereof.

12. The method of claim 10, said method further comprises
a. collecting data of at least two users in said environment.

13. The method of claim 10, said method further comprises communicating a safe egress path and/or a safe body position to multiple users in the environment.

14. The use of the wearable haptic navigation system of claim 1 in law enforcement, emergency medical services and firefighting.

15. The use of the wearable haptic navigation system of claim 1 in actual flight and flight simulation.

16. The wearable haptic navigation system of claim 1 in combination with a flight simulation system.

17. A wearable device comprising:
a wearable garment;
an input module to collect sensory related data;
a plurality of sensory devices connected to the wearable garment that actuate to produce one or more sensory stimulations, each of said one or more sensory stimulations for inducing physiological stimulation; and
a control centre comprising:
a processor for determining sensory events, each of said sensory events defining a synergistic action of said one or more sensory stimulations as a signal pathway to produce one or more sensory outcomes, each of said one or more sensory outcomes for inducing a physiological response or sensory perception;
a transceiver for receiving the sensory related data collected via the input module, and in response, sending an activating signal to actuate one or more of said plurality of sensory devices to activate the sensory events wherein the synergistic action of two or more sensory stimulations comprise at least two of electrical muscle stimulation, audio, haptic feedback, force feedback, constriction/compression, airflow, temperature stimulation and combinations thereof; said wearable device in communication with a flight simulator.
